SEQUENCE LISTING (14 sequences)

SEQ ID NO: 1 - DNA, 24 bp, synthetic construct
  source 1..24: mol_type = other DNA
gaacaaaaaattcctcaaagcttc

SEQ ID NO: 2 - DNA, 22 bp, synthetic construct
  source 1..22: mol_type = other DNA
tcaaaaagatcaagaagaccac

SEQ ID NO: 3 - DNA, 40 bp, synthetic construct
  source 1..40: mol_type = other DNA
aacggtacgacgcgcacgtagagaatatagctggatctgg

SEQ ID NO: 4 - DNA, 38 bp, synthetic construct
  source 1..38: mol_type = other DNA
ttattattatttattatttatatgcgcgtcgtaccgtt

SEQ ID NO: 5 - DNA, 18 bp, synthetic construct
  source 1..18: mol_type = other DNA
aagtccgcctatacgcct

SEQ ID NO: 6 - DNA, 21 bp, synthetic construct
  source 1..21: mol_type = other DNA
catttttgtaattcagaccgg

SEQ ID NO: 7 - DNA, 39 bp, synthetic construct
  source 1..39: mol_type = other DNA
acggcgcaataccagccggaactggtttcatctgattac

SEQ ID NO: 8 - DNA, 38 bp, synthetic construct
  source 1..38: mol_type = other DNA
tttttcttttttgagcgtcttccctggtattgcgccgt

SEQ ID NO: 9 - DNA, 26 bp, synthetic construct
  source 1..26: mol_type = other DNA
cagggaagacgctcaaaaaagaaaaa

SEQ ID NO: 10 - DNA, 26 bp, synthetic construct
  source 1..26: mol_type = other DNA
gttagatgataaattaggtaatgctg

SEQ ID NO: 11 - DNA, 24 bp, synthetic construct
  source 1..24: mol_type = other DNA
cagctgtataaattttaccttcac

SEQ ID NO: 12 - DNA, 43 bp, synthetic construct
  source 1..43: mol_type = other DNA
tgtcgatcgcgttggaaatccacatttaacagataatgttgat

SEQ ID NO: 13 - DNA, 38 bp, synthetic construct
  source 1..38: mol_type = other DNA
tgcgttccgtcgcgtactgacgtccaacgcgatcgaca

SEQ ID NO: 14 - DNA, 28 bp, synthetic construct
  source 1..28: mol_type = other DNA
gttggacgtcagtacgcgacggaacgca

(19) United States
(12) Patent Application Publication
(10) Pub. No.: US 2026/0098290 A1
(43) Pub. Date: Apr. 9, 2026

(54) METHOD FOR DETECTING N TARGET NUCLEIC ACIDS IN SAMPLE USING N DETECTION TEMPERATURES

(71) Applicant: SEEGENE, INC., Seoul (KR)

(72) Inventors: Young Jo LEE, Seoul (KR); Han Bit LEE, Seoul (KR); Hyun Ji HAN, Seoul (KR); Jeong Woo KIM, Seoul (KR)

(73) Assignee: SEEGENE, INC., Seoul (KR)

(21) Appl. No.: 19/404,105

(22) Filed: Dec. 1, 2025

Related U.S. Application Data
(63) Continuation of application No. PCT/KR2024/007542, filed on Jun. 3, 2024.

(30) Foreign Application Priority Data
Jun. 2, 2023 (KR) 10-2023-0071701

(51) Int. Cl. C12Q 1/6825; C12Q 1/686; G16B 40/10

(52) U.S. Cl. CPC C12Q 1/6825; C12Q 1/686; G16B 40/10

(57) ABSTRACT

The present invention relates to a method of detecting n target nucleic acids in a sample, and more particularly, to a method of detecting n target nucleic acids in a sample in a real-time manner in one reaction vessel by being configured such that signals from one or two target nucleic acids are detected at each of n (n is an integer of 3 or more) detection temperatures despite the use of a single fluorescent label.

### CROSS-REFERENCE TO RELATED APPLICATIONS

This application is a Bypass Continuation of International Application No. PCT/KR2024/007542 filed Jun. 3, 2024 and claims priority from Korean Patent Application No. 10-2023-0071701, filed on Jun. 2, 2023, in the Korean Intellectual Property Office, the disclosure of which is incorporated herein by reference in its entirety.

### INCORPORATION BY REFERENCE OF SEQUENCE LISTING

The instant application contains a Sequence Listing which has been filed electronically in xml format and is hereby incorporated by reference in its entirety. Said xml file, created on Nov. 29, 2025, is named Q315658SEQLIST.xml and is 13,501 bytes in size.

### TECHNICAL FIELD

The present invention relates to a method for detecting n target nucleic acids in a sample, more particularly, to a method for detecting n target nucleic acids in a sample using n detection temperatures in a nucleic acid amplification reaction.

### BACKGROUND ART

In order to detect target nucleic acids, real-time detection methods capable of detecting target nucleic acids while monitoring target amplification in a real-time manner have been widely used. The real-time detection methods generally use labeled probes or primers that specifically hybridize with target nucleic acids. Examples of methods using hybridization between labeled probes and target nucleic acids include Molecular beacon method using dual-labeled probes with hairpin structure (see Tyagi et al., Nature Biotechnology v. 14 Mar. 1996), HyBeacon method (see French D J et al., Mol. Cell Probes, 15(6): 363-374 (2001)), Hybridization probe method using two probes labeled each with a donor and an acceptor (see Bernad et al., 147-148 Clin Chem 2000; 46), and Lux method using single-labeled oligonucleotides (see U.S. Pat. No. 7,537,886). TaqMan method using dual-labeled probes and its cleavage by 5′-nuclease activity of DNA polymerase are widely used in the art (see U.S. Pat. Nos. 5,210,015 and 5,538,848).

Examples of methods using labeled primers include Sunrise primer method (see Nazarenko et al., 2516-2521 Nucleic Acids Research, 1997, v. 25 no. 12, and U.S. Pat. No. 6,117,635), Scorpion primer method (see Whitcombe et al., 804-807, Nature Biotechnology v. 17 Aug. 1999 and U.S. Pat. No. 6,326,145), and TSG primer method (see WO 2011-078441).

As alternative approaches, real-time detection methods using duplexes formed dependent on the presence of target nucleic acids have been proposed: Invader analysis (see U.S. Pat. Nos. 5,691,142, 6,358,691, and 6,194,149), PTO cleavage and extension (PTOCE) method (see WO 2012/096523), PTO cleavage and extension-dependent signaling oligonucleotide hybridization (PCE-SH) method (see WO 2013/115442), and PTO cleavage and extension-dependent non-hybridization (PCE-NH) method (see WO 2014/104818).

The conventional real-time detection technologies as described above detect a signal provided from a fluorescent label at a predetermined detection temperature in signal amplification process associated or not associated with target amplification. When a plurality of target nucleic acids are detected using a single type of label in one reaction tube in accordance with any of the conventional real-time detection technologies, signals provided from the plurality of target nucleic acids are not distinguished from each other. Thus, conventional real-time detection techniques generally use different types of labels to detect a plurality of target nucleic acids. Melting analysis using Tm difference can detect a plurality of target nucleic acids even using a single type of label. However, the melting analysis has a serious drawback in that it takes longer time than the real-time techniques, and it becomes more difficult to design probes having different Tm values as the number of target nucleic acids increases.

In order to solve this problem, U.S. Patent Application Publication No. 2017-0247750 or 2019-0024155 discloses a method for determining the presence of two or more target nucleic acids using a single label in a real-time manner without using melting analysis. Specifically, the method as disclosed in the references comprises reacting, in one reaction vessel, a sample with two signal-generating means comprising the same type of labels not distinguishable from each other, measuring signals at two detection temperatures, and analyzing the measured signals to determine the presence of two target nucleic acids.

According to the above method, a combined signal from two target nucleic acids is measured at a first temperature, a single signal from one target nucleic acid is measured at a second temperature, the presence of one target nucleic acid is determined by the signal measured at the second temperature, and the presence of the other target nucleic acid is determined by the difference between the signals measured at the two temperatures. The method further uses a reference value for a more accurate determination of the difference between the signals.

However, the method as disclosed in the references requires a complicated mathematical processing using several reference values to determine the presence of three or more target nucleic acids. In addition, the method requires precise control of the target signals at each detection temperature such that a combined signal from three target nucleic acids is measured at a temperature, a combined signal from two target nucleic acids is measured at another temperature, and a single signal from a target nucleic acid is measured at the other temperature.

Therefore, there is a need to develop a novel method for detecting three or more target nucleic acids in a more convenient and improved manner.

Throughout this specification, a number of patents and documents are referenced and their references are indicated in parentheses. In order to more clearly describe the present disclosure and the level of the technical field to which the present disclosure pertains, the disclosures of these patents and documents are incorporated herein by reference in their entirety.

### DISCLOSURE

### Technical Problem

The present inventors have endeavored to develop a novel method for detecting n target nucleic acids using a single type of label in a single reaction vessel, wherein n is an integer of 3 or more. As a result, the present inventors have found that the presence of the n target nucleic acids can be easily determined by configuring a signal to be provided from one or two target nucleic acids at each of the n detection temperatures even when using a single type of label.

Accordingly, it is an object of the present disclosure to provide a method for detecting n target nucleic acids in a sample using n detection temperatures.

It is another object of the present disclosure to provide a kit for detecting n target nucleic acids in a sample using n detection temperatures.

It is another object of the present disclosure to provide a computer readable storage medium containing instructions to configure a processor to perform a method for detecting n target nucleic acids in a sample using n detection temperatures.

It is another object of the present disclosure to provide a device for detecting n target nucleic acids in a sample using n detection temperatures.

It is another object of the present disclosure to provide a computer program to be stored on a computer readable storage medium to configure a processor to perform a method for detecting n target nucleic acids in a sample using n detection temperatures.

Other objects and advantages of the present disclosure will become more apparent from the following detailed description together with the appended claims and drawings.

### Technical Solution

In an aspect of the present disclosure, there is provided a method of detecting n target nucleic acids in a sample using n detection temperatures, comprising the steps of: (a) reacting, in a single reaction vessel, a sample suspected of containing at least one of the n target nucleic acids with n compositions for detecting the n target nucleic acids, wherein n is an integer of 3 or more, wherein, during the reacting, the n target nucleic acids in the sample are amplified, wherein each of the n compositions comprises one or more oligonucleotides capable of providing a signal depending on the presence of its corresponding target nucleic acid among the n target nucleic acids, and signals provided by the n compositions are not differentiated from each other by a single detection channel, wherein 1 to n−1 of the n compositions are each configured to provide a signal indicative of the presence of its corresponding target nucleic acid only at one different detection temperature among the n detection temperatures in the presence of the corresponding target nucleic acid, whereas the other compositions are each configured to provide a signal indicative of the presence of its corresponding target nucleic acid at two to n adjacent detection temperatures among the n detection temperatures arranged in order in the presence of the corresponding target nucleic acid, wherein at each of the n detection temperatures, signals are provided by one or two of the n compositions; (b) measuring the signals at the n detection temperatures under the single detection channel, wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and (c) determining the presence of a target nucleic acid from the single signal measured at the single-signal detection temperature and determining the presence of a target nucleic acid from an extracted single signal, which is obtained by extraction from the combined signal measured at the combined-signal detection temperature, whereby the presence of the n target nucleic acids are determined from the signals measured at the n detection temperatures.

In certain embodiments, the reacting is real-time PCR.

In certain embodiments, at least one of the n compositions provides a signal in a manner dependent on formation or dissociation of a duplex.

In certain embodiments, at least one of the n compositions provides a signal by formation of a duplex in a manner dependent on cleavage of a mediation oligonucleotide specifically hybridized to its corresponding target nucleic acid.

In certain embodiments, at least one of the n compositions provides a signal in a manner dependent on cleavage of an oligonucleotide specifically hybridized to its corresponding target nucleic acid.

In certain embodiments, each of the n compositions provides a signal in a predetermined temperature range but does not provide a signal in other temperature ranges.

In certain embodiments, n is 3.

In certain embodiments, one of three compositions is configured to provide a signal indicative of the presence of its corresponding target nucleic acid only at one detection temperature among three detection temperatures arranged in order, and the other two compositions are each configured to provide a signal indicative of the presence of its corresponding target nucleic acid at two adjacent detection temperatures among the three detection temperatures arranged in order.

In certain embodiments, one of three compositions is configured to provide a signal indicative of the presence of its corresponding target nucleic acid only at one detection temperature among three detection temperatures arranged in order, another composition is configured to provide a signal indicative of the presence of its corresponding target nucleic acid at two adjacent detection temperatures among the three detection temperatures arranged in order, and the other composition is configured to provide a signal indicative of the presence of its corresponding target nucleic acid at the three detection temperatures arranged in order.

In certain embodiments, two of three compositions are each configured to provide a signal indicative of the presence of its corresponding target nucleic acid only at one different detection temperature among three detection temperatures arranged in order, and the other one composition is configured to provide a signal indicative of the presence of its corresponding target nucleic acid at two or three adjacent detection temperatures among the three detection temperatures arranged in order.

In certain embodiments, n is 4.

In certain embodiments, one of four compositions is configured to provide a signal indicative of the presence of its corresponding target nucleic acid only at one detection temperature among four detection temperatures arranged in order, and the other three compositions are each configured to provide a signal indicative of the presence of its corresponding target nucleic acid at two adjacent detection temperatures among the four detection temperatures arranged in order.

In certain embodiments, one of four compositions is configured to provide a signal indicative of the presence of its corresponding target nucleic acid only at one detection temperature among four detection temperatures arranged in order, another two compositions are each configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three adjacent detection temperatures among the four detection temperatures arranged in order.

In certain embodiments, one of four compositions is configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other two compositions are each configured to provide a signal indicative of the presence of its corresponding target nucleic acid at three adjacent detection temperatures among the four detection temperatures arranged in order.

In certain embodiments, two of four compositions are each configured to provide a signal indicative of the presence of its corresponding target nucleic acid only at one different detection temperature among four detection temperatures arranged in order, and the other two compositions are each configured to provide a signal indicative of the presence of its corresponding target nucleic acid at two or three adjacent detection temperatures among the four detection temperatures arranged in order.

In certain embodiments, two of four compositions are each configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three or four adjacent detection temperatures among the four detection temperatures arranged in order.

In certain embodiments, three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at two to four adjacent detection temperatures among the four detection temperatures arranged in order.

In certain embodiments, n is 5.

In certain embodiments, (i) one of five compositions is configured to provide a signal indicative of the presence of its corresponding target nucleic acid only at one detection temperature among five detection temperatures arranged in order, and the other four compositions are each configured to provide a signal indicative of the presence of its corresponding target nucleic acid at two to five adjacent detection temperatures among the five detection temperatures arranged in order; (ii) two of five compositions are each configured to provide a signal indicative of the presence of its corresponding target nucleic acid only at one different detection temperature among five detection temperatures arranged in order, and the other three compositions are each configured to provide a signal indicative of the presence of its corresponding target nucleic acid at two to five adjacent detection temperatures among the five detection temperatures arranged in order; (iii) three of five compositions are each configured to provide a signal indicative of the presence of its corresponding target nucleic acid only at one different detection temperature among five detection temperatures arranged in order, and the other two compositions are each configured to provide a signal indicative of the presence of its corresponding target nucleic acid at two to five adjacent detection temperatures among the five detection temperatures arranged in order; or (iv) four of five compositions are each configured to provide a signal indicative of the presence of its corresponding target nucleic acid only at one different detection temperature among five detection temperatures arranged in order, and the other one composition is configured to provide a signal indicative of the presence of its corresponding target nucleic acid at two to five adjacent detection temperatures among the five detection temperatures arranged in order.

In certain embodiments, two of the n compositions do not provide a signal indicative of the presence of its corresponding target nucleic acids at two or more identical detection temperatures.

In certain embodiments, the number of the combined-signal detection temperatures is n−2 or less.

In certain embodiments, the extraction of a single signal from the combined signal measured at the combined-signal detection temperature is performed by using (i) the combined signal measured at the combined-signal detection temperature and (ii) a single signal measured at a single-signal detection temperature, which is provided by a composition also providing a signal at the combined signal detection temperature.

In certain embodiments, the extraction of a single signal from the combined signal measured at the combined-signal detection temperature is performed by eliminating a signal provided by another composition from the combined signal measured at the combined-signal detection temperature.

In certain embodiments, the extraction of a single signal from the combined signal measured at the combined-signal detection temperature is performed by (i) the difference between the combined signal measured at the combined-signal detection temperature and a single signal provided by another composition at a single-signal detection temperature and (ii) a reference value.

In certain embodiments, the reference value is a value that reflects the change in signals provided by a composition at two detection temperatures in the presence of its corresponding target nucleic acid.

In certain embodiments, the reference value is obtained by (i) reacting a composition with a sample containing its corresponding target nucleic acid in a reaction vessel different from the reaction vessel of step (a) to amplify the target nucleic acid in the sample, and (ii) measuring signals at two detection temperature, and (iii) calculating the difference between the signals.

In other aspect of the present disclosure, there is provided a kit for detecting n target nucleic acids in a sample using n detection temperatures, comprising: (a) n compositions for detecting the n target nucleic acids, wherein n is an integer of 3 or more, wherein each of the n compositions comprises one or more oligonucleotides capable of providing a signal depending on the presence of its corresponding target nucleic acid among the n target nucleic acids, and signals provided by the n compositions are not differentiated from each other by a single detection channel, wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid, wherein at each of the n detection temperatures, signals are provided by one or two of the n compositions, wherein none to n 1 of the n detection temperatures are single-signal detection temperatures selected such that a single signal is provided by one of the n compositions, whereas the other detection temperatures are combined-signal detection temperatures selected such that a combined signal is provided by two of the n compositions; and (b) an instruction describing the method as described above.

In another aspect of the present disclosure, there is provided a computer readable storage medium containing instructions to configure a processor to perform a method for detecting n target nucleic acids in a sample using n detection temperatures, the method comprising: (a) receiving signals measured at the n detection temperatures under a single detection channel, wherein the signals are obtained by reacting, in a single reaction vessel, a sample suspected of containing at least one of n target nucleic acids with n compositions for detecting n target nucleic acids, and measuring the signals at the n detection temperatures under the single detection channel, wherein n is an integer of 3 or more, wherein, during the reacting, the n target nucleic acids in the sample are amplified, wherein each of the n compositions comprises one or more oligonucleotides capable of providing a signal depending on the presence of its corresponding target nucleic acid among the n target nucleic acids, and signals provided by the n compositions are not differentiated from each other by a single detection channel, wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid, wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and (b) determining the presence of a target nucleic acid from the single signal measured at the single-signal detection temperature and determining the presence of a target nucleic acid from a single signal, which is extracted from the combined signal measured at the combined-signal detection temperature such that the presence of the n target nucleic acids are determined from the signals measured at the n detection temperatures.

In another aspect of the present disclosure, there is provided a device for detecting n target nucleic acids in a sample using n detection temperatures, comprising (a) a computer processor and (b) the computer readable storage medium as described above coupled to the computer processor, where n is an integer of 3 or more.

In another aspect of the present disclosure, there is provided a computer program to be stored on a computer readable storage medium to configure a processor to perform a method for detecting n target nucleic acids in a sample using n detection temperatures, the method comprising: (a) receiving signals measured at the n detection temperatures under a single detection channel, wherein the signals are obtained by reacting, in a single reaction vessel, a sample suspected of containing at least one of n target nucleic acids with n compositions for detecting n target nucleic acids, and measuring the signals at the n detection temperatures under the single detection channel, wherein n is an integer of 3 or more, wherein, during the reacting, the n target nucleic acids in the sample are amplified, wherein each of the n compositions comprises one or more oligonucleotides capable of providing a signal depending on the presence of its corresponding target nucleic acid among the n target nucleic acids, and signals provided by the n compositions are not differentiated from each other by a single detection channel, wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid, wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and (b) determining the presence of a target nucleic acid from the single signal measured at the single-signal detection temperature and determining the presence of a target nucleic acid from a single signal, which is extracted from the combined signal measured at the combined-signal detection temperature such that the presence of the n target nucleic acids are determined from the signals measured at the n detection temperatures.

### Advantageous Effects

The method of the present disclosure is configured such that signals from one or two target nucleic acids are detected at each of n detection temperatures, enabling the determination of the presence of n target nucleic acids in a sample in a real-time manner in one reaction vessel despite the use of a single fluorescent label. The method of the present disclosure does not require post-amplification melting analysis, and therefore can reduce turnaround time compared to conventional melting analysis. In addition, the method of the present disclosure is convenient in mathematical processing due to reduced number of reference values compared to the method described in U.S. Patent Application Publication No. 2017-0247750 or 2019-0024155. In order to determine the presence of three target nucleic acids, the prior art requires that a combined signal from three target nucleic acids be measured at one temperature, another combined signal from two target nucleic acids be measured at another temperature, and a single signal from one target nucleic acid be measured at the other temperature, whereas the method of the present disclosure can be achieved with various embodiments without being bound by such requirement, providing improved flexibility and utility over conventional methods.

### DESCRIPTION OF DRAWINGS

FIG. 1 illustrates a temperature range in which signals are provided by a first composition (60° C. or less; upper bar), a temperature range in which signals are provided by a second composition (50° C. to 75° C.; middle bar), and a temperature range in which signals are provided by a second composition (50° C. to 75° C.; lower bar); and first, second, and third detection temperatures selected within each of the temperature ranges (vertical dotted lines).

FIGS. 2A to 2F show amplification curves at 60° C., 62° C., 70° C., 76° C., and 79° C. for the first composition (Tube 1 containing the first target nucleic acid; Tube 2 containing distilled water), the second composition (Tube 3 containing the second target nucleic acid; Tube 4 containing distilled water), and the third composition (Tube 5 containing the third target nucleic acid; Tube 6 containing distilled water).

FIGS. 3A and 3B show amplification curves at 62° C., 70° C., and 79° C. for the first to third composition (Tube 1 containing CT genomic DNA; Tube 2 containing CT genomic DNA and NG genomic DNA; Tube 3 containing UP genomic DNA; Tube 4 containing NG genomic DNA and UP genomic DNA; Tube 5, negative control).

FIGS. 4A and 4B show amplification curves of each target nucleic acid for Tubes 1 to 5 (Tube 1 containing CT genomic DNA; Tube 2 containing CT genomic DNA and NG genomic DNA; Tube 3 containing UP genomic DNA; Tube 4 containing NG genomic DNA and UP genomic DNA; Tube 5, negative control).

FIGS. 5A and 5B show amplification curves at 60° C., 70° C., and 76° C. for the first to third composition (Tube 1 containing CT genomic DNA; Tube 2 containing CT genomic DNA and NG genomic DNA; Tube 3 containing UP genomic DNA; Tube 4 containing NG genomic DNA and UP genomic DNA; Tube 5, negative control).

FIGS. 6A and 6B show amplification curves of each target nucleic acid for Tubes 1 to 5 (Tube 1 containing CT genomic DNA; Tube 2 containing CT genomic DNA and NG genomic DNA; Tube 3 containing UP genomic DNA; Tube 4 containing NG genomic DNA and UP genomic DNA; Tube 5, negative control).

### BEST MODE

One of the features of the present disclosure is to determine the presence of n target nucleic acids in a sample by a real-time nucleic acid amplification reaction using a single type of label. Conventionally, real-time nucleic acid amplification reactions using different types of labels for each target nucleic acid or post-amplification melting analyses using a single type of label have been performed. In contrast, the method of the present disclosure can determine the presence of n target nucleic acids in a sample by real-time nucleic acid amplification without melting analysis despite the use of a single type of label.

### I. Method for Detecting n Target Nucleic Acids

In an aspect of the present disclosure, there is provided a method for detecting n target nucleic acids in a sample using n detection temperatures, comprising the steps of:
(a) reacting, in a single reaction vessel, a sample suspected of containing at least one of the n target nucleic acids with n compositions for detecting the n target nucleic acids,
wherein n is an integer of 3 or more,
wherein, during the reacting, the n target nucleic acids in the sample are amplified,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are each configured to provide a signal indicative of the presence of its corresponding target nucleic acid only at one different detection temperature among the n detection temperatures in the presence of the corresponding target nucleic acid, whereas the other compositions are each configured to provide a signal indicative of the presence of its corresponding target nucleic acid at two to n adjacent detection temperatures among the n detection temperatures arranged in order in the presence of the corresponding target nucleic acid,
wherein at each of the n detection temperatures, signals are provided by one or two of the n compositions;
(b) measuring the signals at the n detection temperatures under the single detection channel,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(c) determining the presence of a target nucleic acid from the single signal measured at the single-signal detection temperature and determining the presence of a target nucleic acid from an extracted single signal, which is obtained by extraction from the combined signal measured at the combined-signal detection temperature, whereby the presence of the n target nucleic acids are determined from the signals measured at the n detection temperatures.

The method of the present disclosure will be described in detail as follows:

Step (a): Reacting a Sample with Compositions for Detecting Target Nucleic Acids

First, in a single reaction vessel, a sample suspected of containing at least one of the n target nucleic acids is reacted with n compositions for detecting the n target nucleic acids.

### Sample

As used herein, the term “sample” refers to any cell, tissue, or fluid from a biological source, or any other medium that can advantageously be evaluated according to this invention. The sample includes virus, bacteria, tissue, cell, blood, serum, plasma, lymph, milk, urine, feces, ocular fluid, saliva, semen, brain extracts, spinal cord fluid (SCF), appendix, spleen and tonsillar tissue extracts, amniotic fluid, ascitic fluid and non-biological samples (e.g., food and water). In addition, the sample includes natural-occurring nucleic acid molecules isolated from biological sources and synthetic nucleic acid molecules. Further, the sample may be a lysate, an extract or an isolated target nucleic acid itself for a specific specimen.

The methods of the present disclosure are used to detect which of the n target nucleic acids in a sample, i.e., to determine which of the n target nucleic acids is present.

### Target Nucleic Acid

As used herein, the term “target nucleic acid,” “target nucleic acid sequence,” or “target sequence” refers to a nucleic acid sequence to be detected or quantified. The target nucleic acids include those newly produced in a reaction as well as those initially present in a sample.

The target nucleic acid includes any DNA (gDNA and cDNA), RNA molecules, and hybrids thereof (chimeric nucleic acids). The target nucleic acid may be in a double-stranded or single-stranded form. When the nucleic acid as a starting material is double-stranded, it is preferred to render the two strands into a single-stranded or partially single-stranded form. Methods known to separate strands include, but are not limited to, heating, alkali, formamide, urea and glycoxal treatment, enzymatic methods (e.g., helicase action), and binding proteins. For example, strand separation can be achieved by heating the strands at a temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA, random primers or target-specific primers may be used.

The target nucleic acid includes any naturally occurring prokaryotic, eukaryotic (for example, protozoans and parasites, fungi, yeast, higher plants, lower and higher animals, including mammals and humans), viral (for example, Herpes viruses, HIV, influenza virus, Epstein-Barr virus, hepatitis virus, polio virus, etc.), or viroid nucleic acid. The nucleic acid molecule can also be any nucleic acid molecule which has been or can be recombinantly produced or chemically synthesized. Thus, the nucleic acid may or may not be found in nature. The target nucleic acid may include known or unknown sequences.

The n target nucleic acids herein can be nucleic acids from n different organisms, n nucleic acids from the same organism, or combinations thereof.

### Composition for Detecting a Target Nucleic Acid

According to the method of the present disclosure, n compositions, i.e., a first to n^{th} compositions, are used to detect n target nucleic acids, i.e., a first to n^{th} target nucleic acids.

Each of the n compositions is specific for one corresponding target nucleic acid. The expression “each of the n compositions is specific to one corresponding target nucleic acid” means that each of the n compositions is involved in the amplification and/or detection of the one corresponding target nucleic acid but not in the amplification and/or detection of other target nucleic acids. In other words, it means that each of the n compositions interacts with the one corresponding target nucleic acid but does not interact with other target nucleic acids.

Specifically, the n^{th} composition is specific for the n^{th} target nucleic acid, for example, when the first to third compositions are used to determine the presence of the first to third target nucleic acids, the first composition is specific for the first target nucleic acid, the second composition is specific for the second target nucleic acid, and the third composition is specific for the third target nucleic acid.

The n compositions are used together in one reaction, i.e., the n compositions are present together in one reaction solution or reaction vessel.

As used herein, the term “composition for detecting a target nucleic acid” refers to a composition containing components used to detect a target nucleic acid, and is used interchangeably with “composition for a target nucleic acid” or simply “composition”.

Examples of components included in the composition include, but are not limited to, oligonucleotide sets used to amplify or detect a target nucleic acid, labels, nucleic acid polymerases, buffers, polymerase cofactors and deoxyribonucleotide-5-triphosphate, and the like. Optionally, the composition may contain a variety of polynucleotide molecules, reverse transcriptase, uracil DNA glycosylase (UDG), a variety of buffers and reagents, and antibodies or compounds that inhibit nucleic acid polymerase activity. In addition, the composition may contain an oligonucleotide set or reagent required for performing a positive control reaction. The optimal amount of components used in a particular reaction can be readily determined by those skilled in the art who are aware of the benefits of the present disclosure. The components of the composition may be present or stored in one or more vessels before the reaction.

According to the methods of the present disclosure, each of the n compositions comprises one or more oligonucleotides capable of providing a signal depending on the presence of its corresponding target nucleic acid among the n target nucleic acids, and signals provided by the n compositions are not differentiated from each other by a single detection channel.

It is noted that the oligonucleotides are a key component in each of the n compositions, and that various other components may be additionally contained in each of the n compositions.

The composition of the present disclosure may contain various oligonucleotides which are involved in the amplification and detection of a target nucleic acid.

In one embodiment, each of the n compositions comprises an oligonucleotide comprising a hybridizing nucleotide sequence complementary to a target nucleic acid.

In an embodiment, each of the n compositions comprises a label, e.g., a fluorescent label, wherein the label included in the composition is the same type of label, i.e., a single type of label.

In an embodiment, each of the n compositions may comprise a primer that serves to amplify a target nucleic acid and a probe that serves to provide a signal dependent on the presence of the target nucleic acid.

As used herein, the term “primer” refers to an oligonucleotide that can serve as an initiation point for synthesis under conditions in which synthesis of a primer extension product complementary to a target nucleic acid sequence (template) is induced, i.e., in the presence of nucleotides and a polymerization agent such as DNA polymerase, and at a suitable temperature and pH. The primer should be long enough to prime the synthesis of the extension product in the presence of a polymerization agent. The suitable length of the primer is determined based on several factors, such as temperature, application, and source of the primer.

As used herein, the term “probe” means a single-stranded nucleic acid molecule comprising a region or regions that are substantially complementary to a target nucleic acid sequence or a nucleic acid sequence derived therefrom. In one embodiment, the 3′-end of the probe is “blocked” to prevent its extension. Blocking can be accomplished according to conventional methods. For example, blocking may be carried out by adding a chemical moiety such as a biotin, a label, a phosphate group, an alkyl group, a non-nucleotide linker, a phosphorothioate or an alkane-diol moiety to the 3′-hydroxyl group of the last nucleotide. Alternatively, blocking may be carried out by removing the 3′-hydroxyl group of the last nucleotide, or by using nucleotides that are free of the 3′-hydroxyl group, such as dideoxynucleotides.

As used herein, the term “complementary” means that primers or probes are sufficiently complementary to hybridize selectively to a target nucleic acid under predetermined annealing conditions or hybridization conditions, encompassing the terms “substantially complementary” and “perfectly complementary”, preferably perfectly complementary.

As used herein, the term “substantially complementary” means that an oligonucleotide is sufficiently complementary to hybridize to a template nucleic acid under predetermined annealing conditions or hybridization conditions, such that the annealed oligonucleotide can be extended by a polymerase to form a complementary copy of the template. Accordingly, this term has a different meaning from the term “completely complementary” or related terms.

As used herein, the term “noncomplementary” means that primers or probes are sufficiently non-complementary not to hybridize selectively to a target nucleic acid under predetermined annealing conditions or hybridization conditions, encompassing the terms “substantially noncomplementary” and “perfectly noncomplementary”, preferably perfectly noncomplementary.

The primer or probe may be single-stranded. The primer or probe includes deoxyribonucleotide, ribonucleotide, or a combination thereof. The primer or probe used in the present disclosure may be composed of naturally occurring dNMP (i.e., dAMP, dGMP, dCMP, and dTMP), modified nucleotide, or non-natural nucleotide.

As used herein, the term “annealing” or “priming” refers to the apposition of an oligodeoxynucleotide or nucleic acid to a template nucleic acid, whereby the apposition enables the polymerase to polymerize nucleotides into a nucleic acid molecule which is complementary to the template nucleic acid or a portion thereof.

As used herein, the term “hybridization” refers to the formation of a double-strand under certain hybridization conditions by means of non-covalent bonding between complementary nucleotide sequences of two single-stranded polynucleotides.

There is no intended distinction between the terms “annealing” and “hybridization”, and these terms will be used interchangeably herein.

In an embodiment, when an oligonucleotide (e.g., probe or primer) hybridized with the target nucleic acid is cleaved to release a fragment, the composition according to the present disclosure may further include a capture oligonucleotide specifically hybridized with the fragment; when the fragment hybridized with the capture oligonucleotide is extended to form an extended strand, the composition according to the present disclosure may further include an oligonucleotide specifically hybridized with the extended strand; the composition according to the present disclosure may further include an oligonucleotide specifically hybridized with the capture oligonucleotide; or the composition according to the present disclosure may include a combination thereof.

As described above, when cleavage of the oligonucleotide is required, the composition according to the present disclosure may include 5′-nuclease and 3′-nuclease, particularly nucleic acid polymerase having 5′-nuclease activity, nucleic acid polymerase having 3′-nuclease activity, or FEN nuclease.

A label herein may be linked to an oligonucleotide or may exist in a free form. The label can be incorporated into extended products during an extension reaction.

### Nucleic Acid Amplification Reaction

According to the methods of the present disclosure, during the reacting, the n target nucleic acids in the sample are amplified.

The reaction according to the methods of the present disclosure is a nucleic acid amplification reaction for amplifying the n target nucleic acids (if present) in a sample.

Specifically, the nucleic acid amplification reaction is a reaction for the n target nucleic acids. More specifically, the nucleic acid amplification reaction is a reaction for simultaneous amplification of the n target nucleic acids in one reaction vessel.

In one embodiment, the nucleic acid amplification reaction is a real-time amplification reaction.

In one embodiment, the nucleic acid amplification reaction is a real-time polymerase chain reaction (real-time PCR).

Polymerase chain reaction is widely used in the art to amplify a target nucleic acid, and includes repeating cycles consisting of denaturation of the target nucleic acid, annealing (hybridization) between the target nucleic acid and a primer, and primer extension (U.S. Pat. Nos. 4,683,195, 4,683,202, and 4,800,159; Saiki et al., (1985) Science 230, 1350-1354).

When the target nucleic acid is double-stranded, it is preferred to render the double strands into a single-stranded or partially single-stranded form. Methods for separating double strands include, but are not limited to, heating, alkaline, formamide, urea and glycoxal treatment, enzymatic methods (e.g., helicase action), and binding proteins. For example, strand separation can be achieved by heating at a temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

The annealing of a primer to a target nucleic acid may be carried out under suitable hybridization conditions as routinely determined by optimization procedures. Conditions such as temperature, concentration of components, hybridization and washing times, buffer components, and their pH and ionic strength may vary depending on various factors, including the lengths and GC contents of oligonucleotide (primer) and the target nucleotide. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

Primers annealed to the target nucleic acid are extended by a template-dependent polymerase, which includes the “Klenow” fragment of E. coli DNA polymerase I, thermostable DNA polymerase, and bacteriophage T7 DNA polymerase. In one embodiment, the template-dependent polymerase is a thermostable DNA polymerase obtained from various bacterial species.

When performing the polymerization reaction, components necessary for the reaction may be provided in excess amounts to the reaction vessel. With respect to the components of the extension reaction, the excess amount refers to an amount of each component such that the ability to achieve the expected extension is not substantially limited by the concentration of the component. It is preferred to provide necessary cofactors such as Mg²⁺, dATP, dCTP, dGTP and dTTP, in a sufficient amount to the reaction mixture in order for the desired reaction to take place.

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA, random primers or target-specific primers may be used.

In another embodiment, ligase chain reaction (LCR, see Wiedmann M, et al., “Ligase chain reaction (LCR)-overview and applications.” PCR Methods and Applications 1994 February; 3(4):S51-64), gap filling LCR (GLCR, see WO 90/01069, EP 439182 and WO 93/00447), Q-beta replicase amplification (Q-beta, see Cahill P, et al., Clin Chem., 37 (9): 1482-5 (1991), U.S. Pat. No. 5,556,751), strand displacement amplification (SDA, see G T Walker et al., Nucleic Acids Res. 20 (7): 16911696 (1992), EP 497272), nucleic acid sequence-based amplification (NASBA, see Compton, J. Nature 350 (6313): 912 (1991)), transcription-mediated amplification (TMA, see Hofmann W P et al., J Clin Virol. 32 (4): 289-93 (2005); U.S. Pat. No. 5,888,779), rolling circle amplification (RCA, see Hutchison C. A. et al., Proc. Natl Acad. Sci. USA. 102:1733217336 (2005), Recombinase polymerase amplification (RPA), or Loop-mediated isothermal amplification (LAMP) may be used to amplify the target nucleic acid, but is not limited thereto.

The amplification methods described above may amplify target nucleic acids through repeating a series of reactions with or without changing temperatures. The unit of amplification comprising the repetition of a series of reactions is expressed as a “cycle.” The unit of cycles may be expressed as the number of the repetition or time being dependent on amplification methods.

For example, the detection of signals may be performed at each cycle, selected several cycles, or end-point of amplification reactions. According to an embodiment, where signals are detected at at least two cycles, the detection of signal in each cycle may be performed at all detection temperatures or some selected detection temperatures.

In an embodiment of the disclosure, nucleic acid polymerases having nuclease activity (e.g., 5′ nuclease activity or 3′ nuclease activity) may be used. In another embodiment of the present disclosure, nucleic acid polymerase having no nuclease activity may be used.

The nucleic acid polymerase useful in the present invention is a thermostable DNA polymerase obtained from a variety of bacterial species, including Thermus aquaticus (Taq), Thermus thermophilus (Tth), Thermus filiformis, Thermis flavus, Thermococcus literalis, Thermus antranikianii, Thermus caldophilus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus, and Aquifex aeolieus. Particularly, the thermostable DNA polymerase is Taq polymerase.

In an embodiment of the present disclosure, amplification of the target nucleic acid is accomplished by an asymmetric PCR. The ratio of primers may be selected in consideration of cleavage or hybridization of downstream oligonucleotides.

According to the present disclosure, in order to provide a signal for a target nucleic acid, a composition for detecting the target nucleic acid is used. The composition provides a signal by reaction with a corresponding target nucleic acid, and the presence of the target nucleic acid is determined by the provided signal.

In an embodiment of the present disclosure, the nucleic acid amplification reaction is performed under conditions such that amplification of target nucleic acids as well as provision of a signal by a composition for detecting a target nucleic acid are done.

In an embodiment, the provision of a signal includes “signal generation or extinction” or “signal increase or decrease”. The provision of a signal herein means that a significant signal, i.e., a signal indicative of the presence of a target nucleic acid, is provided. For example, a significant signal, i.e., a signal indicative of the presence of a target nucleic acid, refers to a signal having an intensity that exceeds a background signal intensity or a signal intensity in the absence of the target nucleic acid. Or a significant signal, i.e., a signal indicative of the presence of a target nucleic acid, refers to a signal having an intensity after subtracting a background signal intensity or a signal intensity in the absence of the target nucleic acid from the intensity of the provided signal.

The provision of a signal herein is interpreted as the provision of a change in a signal, and the provision of a change in a signal refers to the provision of a change in a signal in the presence of a target nucleic acid relative to a signal in the absence of the target nucleic acid.

### Signal Provision Mechanism

According to the method of the present disclosure, each of the n compositions may provide a signal by any of various signal generation mechanisms known in the art.

In one embodiment, at least one of the n compositions provides a signal in a manner dependent on formation or dissociation of a duplex.

As used herein, the expression “providing a signal in a manner dependent on formation or dissociation of a duplex” means that a detectable signal is provided dependent on the association or dissociation of two nucleic acid molecules. The expression encompasses providing a signal by a duplex (e.g., a duplex between a detection oligonucleotide having a label and a target nucleic acid) formed depending on the presence of the target nucleic acid. Alternatively, the expression may encompass providing a signal by inhibition of hybridization of a duplex (e.g., a duplex between a detection oligonucleotide having a label and a target nucleic acid), or providing a signal by dissociation of a duplex that is released by cleavage depending on the presence of the target nucleic acid.

The term “association” or “dissociation” has the same meaning as the term “hybridization” or “denaturation”.

In an embodiment, the duplex comprises a double-stranded nucleic acid molecules.

In an embodiment, the composition does not provide a signal by cleavage of an oligonucleotide in a manner independent of the formation or dissociation of a duplex. For example, when a fragment cleaved from an oligonucleotide is not involved in the formation or dissociation of a duplex, the cleavage of the oligonucleotide alone does not provide a signal.

As used herein, the term “detection oligonucleotide” is an oligonucleotide that is involved in the provision of a detectable signal. In an embodiment, the detection oligonucleotide encompasses an oligonucleotide involved in actual signal provision. For example, the signal provision depends on hybridization or non-hybridization of a detection oligonucleotide with another oligonucleotide (e.g., a target nucleic acid or an oligonucleotide comprising a nucleotide sequence complementary to the detection oligonucleotide).

In an embodiment, the detection oligonucleotide comprises at least one label.

The signal by the formation of a duplex between a target nucleic acid and the detection oligonucleotide may be provided by various methods, including Scorpion method (Whitcombe et al, Nature Biotechnology 17:804-807 (1999)), Sunrise (or Amplifluor) method (Nazarenko et al, Nucleic Acids Research, 25(12):2516-2521 (1997), and U.S. Pat. No. 6,117,635), Lux method (U.S. Pat. No. 7,537,886), Plexor method (Sherrill C B, et al., Journal of the American Chemical Society, 126:4550-45569 (2004)), Molecular Beacon method (Tyagi et al, Nature Biotechnology v. 14 Mar. 1996), HyBeacon method (French D J et al., Mol. Cell Probes, 15(6):363-374 (2001)), adjacent hybridization probe method (Bernard, P. S. et al., Anal. Biochem., 273:221(1999)), and LNA method (U.S. Pat. No. 6,977,295).

In certain embodiments, at least one of the n compositions provides a signal by formation of a duplex in a manner dependent on cleavage of a mediation oligonucleotide specifically hybridized to its corresponding target nucleic acid.

As used herein, the term used herein “mediation oligonucleotide” is an oligonucleotide which mediates production of a duplex not containing a target nucleic acid sequence.

In an embodiment, the cleavage of the mediation oligonucleotide per se does not provide a signal and a fragment formed by hybridization and cleavage of the mediation oligonucleotide is involved in successive reactions for signal provision.

In an embodiment, the hybridization or cleavage of the mediation oligonucleotide per se does not provide a signal.

In an embodiment of the present disclosure, the mediation oligonucleotide includes an oligonucleotide which is hybridized with a target nucleic acid and cleaved to release a fragment, leading to mediate the production of a duplex. Particularly, the fragment mediates the production of a duplex by an extension of the fragment on a capture oligonucleotide.

In an embodiment of the present disclosure, the mediation oligonucleotide comprises (i) a targeting portion comprising a hybridizing nucleotide sequence complementary to the target nucleic acid sequence and (ii) a tagging portion comprising a nucleotide sequence non-complementary to the target nucleic acid sequence.

In an embodiment of the present disclosure, the cleavage of the mediation oligonucleotide release a fragment and the fragment is specifically hybridized with a capture oligonucleotide and extended on the capture oligonucleotide.

In an embodiment of the present disclosure, a mediation oligonucleotide hybridized with a target nucleic acid is cleaved to release a fragment and the fragment is specifically hybridized with a capture oligonucleotide and the fragment is extended to form an extended strand, resulting in formation of an extended duplex between the extended stand and the capture oligonucleotide providing a signal indicating the presence of the target nucleic acid.

In an embodiment of the present disclosure, where a third oligonucleotide comprising a hybridizing nucleotide sequence complementary to the extended strand is used, the hybridization of the third oligonucleotide and the extended strand forms other type of a duplex providing a signal indicating the presence of the target nucleic acid.

In an embodiment of the present disclosure, where a third oligonucleotide comprising a hybridizing nucleotide sequence complementary to the capture oligonucleotide is used, the formation of a duplex between the third oligonucleotide and the capture oligonucleotide is inhibited by the formation of the duplex between the extended strand and the capturing oligonucleotide, leading to provision of a signal indicating the presence of the target nucleic acid.

In an embodiment of the present disclosure, the fragment, the extended strand, the capture oligonucleotide, the third oligonucleotide, or combinations thereof can serve as a detection oligonucleotide.

The signal by the duplex formed in a dependent manner on cleavage of the mediation oligonucleotide may be provided by various methods, including PTOCE (PTO cleavage and extension) method (WO 2012/096523), PCE-SH (PTO Cleavage and Extension-Dependent Signaling Oligonucleotide Hybridization) method (WO 2013/115442) and PCE-NH (PTO Cleavage and Extension-Dependent Non-Hybridization) method (WO 2014/104818).

With referring to terms disclosed in the above references, the corresponding examples of the oligonucleotides are as follows: a mediation oligonucleotide corresponds to a PTO (Probing and Tagging Oligonucleotide), a capture oligonucleotide to a CTO (Capturing and Templating Oligonucleotide), and a third oligonucleotide to SO (Signaling Oligonucleotide) or HO (Hybridization Oligonucleotide), respectively. SO, HO, CTO, extended strand or their combination can take a role as a detection oligonucleotide.

Where the signal by the duplex formed in a dependent manner on cleavage of the mediation oligonucleotide is provided by the PTOCE method, the composition according to the present disclosure comprises an upstream oligonucleotide; a PTO (Probing and Tagging Oligonucleotide) comprising a hybridizing nucleotide sequence complementary to the target nucleic acid sequence; a CTO (Capturing and Templating Oligonucleotide); suitable label; and a template-dependent nucleic acid polymerase having 5′ nuclease activity. The PTO comprises (i) a 3′-targeting portion comprising a hybridizing nucleotide sequence complementary to the target nucleic acid sequence and (ii) a 5′-tagging portion comprising a nucleotide sequence non-complementary to the target nucleic acid sequence. The CTO comprises in a 3′ to 5′ direction (i) a capturing portion comprising a nucleotide sequence complementary to the 5′-tagging portion or a part of the 5′-tagging portion of the PTO and (ii) a templating portion comprising a nucleotide sequence non-complementary to the 5′-tagging portion and the 3′-targeting portion of the PTO.

The particular example of the signal provision by the PTOCE method comprises the steps of:

(a) hybridizing the target nucleic acid sequence with the upstream oligonucleotide and the PTO; (b) contacting the resultant of the step (a) to an enzyme having a 5′ nuclease activity under conditions for cleavage of the PTO; wherein the upstream oligonucleotide or its extended strand induces cleavage of the PTO by the enzyme having the 5′ nuclease activity such that the cleavage releases a fragment comprising the 5′-tagging portion or a part of the 5′-tagging portion of the PTO; (c) hybridizing the fragment released from the PTO with the CTO; wherein the fragment released from the PTO is hybridized with the capturing portion of the CTO; and (d) performing an extension reaction using the resultant of the step (c) and a template-dependent nucleic acid polymerase; wherein the fragment hybridized with the capturing portion of the CTO is extended and an extended duplex is formed; wherein the extended duplex has a Tm value adjustable by (i) a sequence and/or length of the fragment, (ii) a sequence and/or length of the CTO or (iii) the sequence and/or length of the fragment and the sequence and/or length of the CTO; wherein the extended duplex provides a target signal by (i) at least one label linked to the fragment and/or the CTO, (ii) a label incorporated into the extended duplex during the extension reaction, (iii) a label incorporated into the extended duplex during the extension reaction and a label linked to the fragment and/or the CTO, or (iv) an intercalating label; and (e) detecting the extended duplex by measuring the target signal at a predetermined temperature such that the extended duplex maintains its double-stranded form, whereby the presence of the extended duplex indicates the presence of the target nucleic acid sequence. In this case, the method further comprises repeating all or some of the steps (a)-(e) with denaturation between repeating cycles.

In the phrase “denaturation between repeating cycles”, the term “denaturation” means to separate a double-stranded nucleic acid molecule into single-stranded nucleic acid molecules.

In step (a) of the PTOCE method, a primer set for amplification of the target nucleic acid may be used instead of the upstream oligonucleotide. In this case, the method further comprises repeating all or some of the steps (a)-(e) with denaturation between repeating cycles.

The PTOCE method can be classified as a process in which the PTO fragment hybridized with the CTO is extended to form an extended strand and the extended strand is then detected. The PTOCE method is characterized in that the formation of the extended strand is detected by using the duplex between the extended strand and the CTO.

There is another approach to detect the formation of the extended strand. For example, the formation of the extended strand may be detected by using an oligonucleotide specifically hybridized with the extended strand (e.g., PCE-SH method). In this method, the signal may be provided from (i) a label linked to the oligonucleotide specifically hybridized with the extended strand, (ii) a label linked to the oligonucleotide specifically hybridized with the extended strand and a label linked to the PTO fragment, (iii) a label linked to the oligonucleotide specifically hybridized with the extended strand and a label incorporated into the extended strand during the extension reaction, or (iv) a label linked to the oligonucleotide specifically hybridized with the extended strand and an intercalating dye. Alternatively, the signal may be provided from (i) a label linked to the extended strand or (ii) an intercalating dye.

Alternatively, the detection of the formation of the extended strand is performed by another method in which inhibition of the hybridization between the CTO and an oligonucleotide being specifically hybridizable with the CTO is detected (e.g., PCE-NH method). Such inhibition is considered to be indicative of the presence of a target nucleic acid. The signal may be provided from (i) a label linked to the oligonucleotide being hybridizable with the CTO, (ii) a label linked to the CTO, (iii) a label linked to the oligonucleotide being hybridizable with the CTO and a label linked to the CTO, or (iv) an intercalating label.

In an embodiment, the oligonucleotide being specifically hybridizable with the CTO has an overlapping sequence with the PTO fragment.

In an embodiment, the detection oligonucleotide includes the oligonucleotide being specifically hybridizable with the extended strand (e.g., PCE-SH method) and oligonucleotide being specifically hybridizable with the CTO (e.g., PCE-NH method). In an embodiment, the detection oligonucleotide includes the extended strand produced during a reaction, or CTO.

The PTOCE-based methods commonly involve the formation of the extended strand depending on the presence of a target nucleic acid sequence. The term “PTOCE-based method” is used herein to intend to encompass various methods for providing signals comprising the formation of an extended strand through cleavage and extension of PTO.

The example of signal generation by the PTOCE-based methods comprises the steps of: (a) hybridizing the target nucleic acid sequence with the upstream oligonucleotide and the PTO; (b) contacting the resultant of the step (a) to an enzyme having 5′ nuclease activity under conditions for cleavage of the PTO; wherein the upstream oligonucleotide or its extended strand induces cleavage of the PTO by the enzyme having 5′ nuclease activity such that the cleavage releases a fragment comprising the 5′-tagging portion or a part of the 5′-tagging portion of the PTO; (c) hybridizing the fragment released from the PTO with the CTO; wherein the fragment released from the PTO is hybridized with the capturing portion of the CTO; (d) performing an extension reaction using the resultant of the step (c) and a template-dependent nucleic acid polymerase; wherein the fragment hybridized with the capturing portion of the CTO is extended to form an extended strand; and (e) detecting the formation of the extended strand by detecting signal provided dependent on the presence of the extended strand. In the step (a), a primer set for amplification of the target nucleic acid sequence may be used instead of the upstream oligonucleotide. In this case, the method further comprises repeating all or some of the steps (a)-(e) with denaturation between repeating cycles.

In an embodiment, the signal provided by the formation of a duplex includes signals induced by hybridization of the duplex (e.g., hybridization of the duplex per se, or hybridization of a third oligonucleotide) or by inhibition of hybridization of a third oligonucleotide due to the formation of a duplex.

In another embodiment, at least one of the n compositions provides a signal in a manner dependent on cleavage of an oligonucleotide specifically hybridized to its corresponding target nucleic acid.

Particularly, the signal is provided by hybridization of the detection oligonucleotide with a target nucleic acid sequence and then cleavage of the detection oligonucleotide.

The signal by hybridization of the detection oligonucleotide with a target nucleic acid sequence and then cleavage of the detection oligonucleotide may be provided by various methods, including TaqMan probe method (U.S. Pat. Nos. 5,210,015 and 5,538,848).

Where the signal is provided by the TaqMan probe method, the composition according to the present disclosure includes a primer set for amplification of a target nucleic acid sequence, a TaqMan probe having a suitable label (e.g., interactive dual label) and a nucleic acid polymerase having 5′-nuclease activity. The TaqMan probe hybridized with a target nucleic acid sequence is cleaved during target amplification and generates signal indicating the presence of the target nucleic acid.

The particular example providing signal by the TaqMan probe method comprises the step of: (a) hybridizing the primer set and the TaqMan probe having a suitable label (e.g., interactive dual label) with the target nucleic acid sequence; (b) amplifying the target nucleic acid sequence by using the resultant of the step (a) and a nucleic acid polymerase having 5′-nuclease activity, wherein the TaqMan probe is cleaved to release the label; and (c) detecting a signal provision from the released label.

Particularly, the signal is provided by cleavage of the detection oligonucleotide in a dependent manner on cleavage of a mediation oligonucleotide specifically hybridized with the target nucleic acid.

In an embodiment, where a mediation oligonucleotide hybridized with target nucleic acid is cleaved to release a fragment, the fragment is specifically hybridized with a detection oligonucleotide and the fragment induces the cleavage of the detection oligonucleotide.

In an embodiment, where a mediation oligonucleotide hybridized with target nucleic acid sequences is cleaved to release a fragment, the fragment is extended to cleave a detection oligonucleotide comprising a hybridizing nucleotide sequence complementary to the capture oligonucleotide.

The signal by cleavage of the detection oligonucleotide in a dependent manner on cleavage of the mediation oligonucleotide may be provided by various methods, including Invader assay (U.S. Pat. No. 5,691,142), PCEC (PTO Cleavage and Extension-Dependent Cleavage) method (WO 2012/134195) and a method described in U.S. Pat. No. 7,309,573. In particular, the method described in U.S. Pat. No. 7,309,573 may be considered as one of PTOCE-based methods using signal provision by cleavage, and in the method, the formation of the extended strand may be detected by detecting cleavage of an oligonucleotide specifically hybridized with the CTO by the formation of the extended strand. Invader assay forms a fragment by cleavage of a mediation oligonucleotide and induces successive cleavage reactions with no extension of the fragment.

In an embodiment of the present disclosure, where the signal is generated in a dependent manner on cleavage of a detection oligonucleotide, the cleavage of the detection oligonucleotide induces signal changes or releases a labeled fragment to be detected.

In an embodiment, the provision of the signal in a manner dependent on the cleavage of the detection oligonucleotide may be employed for a composition according to the present disclosure which is configured to provide a signal indicative of the presence of its corresponding target nucleic acid at two or n adjacent detection temperatures among the n detection temperatures in order in the presence of the corresponding target nucleic acid, as described below. Where the signal is provided by cleavage of the detection oligonucleotide, a released label by the cleavage may be detected at any temperatures. Therefore, such signal provision mechanism cannot be employed for a composition according to the present disclosure which is configured to provide a signal indicative of the presence of its corresponding target nucleic acid only at one different detection temperature among the n detection temperatures in the presence of the corresponding target nucleic acid, but can be employed for a composition according to the present disclosure which is configured to provide a signal at two or more, e.g., all of n detection temperatures.

In an embodiment, the detection oligonucleotide comprises at least one label. In an embodiment, the detection oligonucleotide may consist of at least one oligonucleotide.

In an embodiment, where the detection oligonucleotide is composed of a plurality of oligonucleotides, it may have a label in various manners. For instance, one of a plurality of oligonucleotides may have at least one label, all of a plurality of oligonucleotides may have at least one label, or one portion of an oligonucleotide may have at least one label and the other portion may not have a label.

### Signal and Label

According to the method of the present disclosure, detection of a signal is performed using a single detection channel.

As used herein, the term “single detection channel” means a detection means for a single type of signal. In a detector comprising several channels (e.g., photodiodes) for several different types of signals, each channel (e.g., photodiode) corresponds to a “single detection channel”.

Herein, each of the n compositions comprises the same type of label, e.g., a fluorescent label, so that the signals provided therefrom are not differentiated from each other by the single detection channel.

The term “signals that are not differentiated from each other by a single detection channel” means that signals are not distinguished from each other by a single detection channel due to their identical or substantially identical signal properties (e.g., optical properties, emission wavelengths, and electrical signals). For example, where the same label (e.g., FAM) is used to detect three target nucleic acids and a single detection channel is used to detect the emission wavelength from the label, the single detection channel does not differentiate signals from the label.

Labels useful in the present disclosure may include various labels known in the art. For example, the label may include, without limitation, a single label, an interactive dual label, an intercalating dye, and an incorporating label.

The single label includes, for example, a fluorescent label, a luminescent label, a chemiluminescent label, an electrochemical label and a metal label. According to an embodiment, the single label provides a different signal (e.g., different signal intensities) depending on its presence on a double strand or single strand. According to an embodiment, the single label is a fluorescent label. The preferable types and binding sites of single fluorescent labels used in this invention are disclosed U.S. Pat. Nos. 7,537,886 and 7,348,141, the teachings of which are incorporated herein by reference in their entirety. For example, the single fluorescent label includes JOE, FAM, TAMRA, ROX and fluorescein-based label. The single label may be linked to oligonucleotides by various methods. For instance, the label is linked to probes through a spacer containing carbon atoms (e.g., 3-carbon spacer, 6-carbon spacer, or 12-carbon spacer).

As a representative of the interactive label system, the FRET (fluorescence resonance energy transfer) label system includes a fluorescent reporter molecule (donor molecule) and a quencher molecule (acceptor molecule). In FRET, the energy donor is fluorescent, but the energy acceptor may be fluorescent or non-fluorescent. In another form of interactive label systems, the energy donor is non-fluorescent, e.g., a chromophore, and the energy acceptor is fluorescent. In yet another form of interactive label systems, the energy donor is luminescent, e.g., bioluminescent, chemiluminescent, electrochemiluminescent, and the acceptor is fluorescent. The interactive label system includes a dual label based on “on contact-mediated quenching” (Salvatore et al., Nucleic Acids Research, 2002 (30) no. 21 e122 and Johansson et al., J. AM. CHEM. SOC 2002 (124) pp 6950-6956). The interactive label system includes any label system in which signal change is induced by interaction between at least two molecules (e.g., dye).

The reporter molecule and the quencher molecule useful in the present invention may include any molecules known in the art. Examples of those are: Cy2™ (506), YO-PRO™-1 (509), YOYO™-1 (509), Calcein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red (615), Nile Red (628), YO-PRO™-3 (631), YOYO™-3 (631), R-phycocyanin (642), C-Phycocyanin (648), TO-PRO™-3 (660), TOTO3 (660), DID DiIC (5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Fluorescein-C3 (520), Pulsar 650 (566), Quasar 570 (667), Quasar 670 (705) and Quasar 705 (610). The numeric in parenthesis is a maximum emission wavelength in nanometer. Preferably, the reporter molecule and the quencher molecule include JOE, FAM, TAMRA, ROX and fluorescein-based label.

Suitable fluorescence molecule and suitable pairs of reporter-quencher are disclosed in a variety of publications as follows: Pesce et al., editors, Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2^{nd} Edition (Academic Press, New York, 1971); Griffiths, Color AND Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); Haugland, R. P., Handbook of Fluorescent Probes and Research Chemicals, 6th Edition (Molecular Probes, Eugene, Oreg., 1996) U.S. Pat. Nos. 3,996,345 and 4,351,760.

It is noteworthy that a non-fluorescent quencher molecule (e.g., black quencher or dark quencher) capable of quenching a fluorescence of a wide range of wavelengths or a specific wavelength may be used in the present invention.

In the signaling system comprising the reporter and quencher molecules, the reporter encompasses a donor of FRET and the quencher encompasses the other partner (acceptor) of FRET. For example, a fluorescein dye is used as the reporter and a rhodamine dye as the quencher.

The interactive dual label may be linked to one strand of a duplex. Where the strand containing the interactive dual label leaves in a single stranded state, it forms a hairpin or random coil structure to induce quenching between the interactive dual label. Where the strand forms a duplex, the quenching is relieved.

The term “hairpin” refers to a structure of an oligonucleotide having a double-stranded “stem” region and a single-stranded “loop” region.

One of the respective interactive dual labels may be linked to one strand of the duplex, and the other of the interactive dual labels may be linked to the other strand of the duplex. The formation of the duplex induces quenching and denaturation of the duplex induces unquenching. Alternatively, where one of the two stands is cleaved, the unquenching may be induced.

Exemplified intercalating dyes useful in this invention include SYBR™ Green I, PO-PRO™-1, BO-PRO™-1, SYTO™43, SYTO™44, SYTO™45, SYTOX™Blue, POPO™-1, POPO™-3, BOBO™-1, BOBO™-3, LO-PRO™-1, JO-PRO™-1, YO-PRO™1, TO-PRO™1, SYTO™11, SYTO™13, SYTO™15, SYTO™16, SYTO™20, SYTO™23, TOTO™-3, YOYO™M3, GelStar™ and thiazole orange. The intercalating dyes intercalate specifically into double-stranded nucleic acid molecules to provide signals.

The incorporating label may be used in a process to generate signals by incorporating a label during primer extension (e.g., Plexor method, Sherrill C B, et al., Journal of the American Chemical Society, 126:4550-45569 (2004)).

The incorporating label may be generally linked to nucleotides. The nucleotide having a non-natural base may be also used.

The term used herein “non-natural base” refers to derivatives of natural bases such as adenine (A), guanine (G), thymine (T), cytosine (C) and uracil (U), which are capable of forming hydrogen-bonding base pairs. The term used herein “non-natural base” includes bases having different base pairing patterns from natural bases as mother compounds, as described, for example, in U.S. Pat. Nos. 5,432,272, 5,965,364, 6,001,983, and 6,037,120. The base pairing between non-natural bases involves two or three hydrogen bonds as natural bases. The base pairing between non-natural bases is also formed in a specific manner. Specific examples of non-natural bases include the following bases in base pair combinations: iso-C/iso-G, iso-dC/iso-dG, K/X, H/J, and M/N (see U.S. Pat. No. 7,422,850).

In an embodiment, at least one of the n compositions may comprise (a) an oligonucleotide comprising a targeting portion comprising a hybridizing nucleotide sequence complementary to a target nucleic acid sequence; and (b) a label.

In an embodiment, at least one of the n compositions may provide a signal in a predetermined temperature range by hybridization of the oligonucleotide with a target nucleic acid.

Where the composition provides a signal in a manner dependent on the formation of a duplex, e.g., hybridization of an oligonucleotide with a target nucleic acid, the temperature range in which the signal is provided may vary depending on the Tm value of the duplex. For example, where a signal is provided by an oligonucleotide (e.g., an oligonucleotide in the form of a molecular beacon probe) that specifically hybridizes to a target nucleic acid, designing the oligonucleotide to have a high Tm value may shift the temperature range in which the signal is providable toward a higher temperature range, whereas designing the oligonucleotide to have a low Tm value may shift the temperature range in which the signal is providable toward a lower temperature range.

In an embodiment, the label included in the composition according to the present disclosure may be interactive dual labels comprising one reporter molecule and one quencher molecule.

The interactive dual labels may be linked to either strand of the duplex. When the strand comprising the interactive dual labels is present in a single-stranded state, the strand forms a hairpin or random coil structure to induce quenching of the interactive double labels. When the strand forms the duplex, the quenching decreases.

In an embodiment, in the presence of a target nucleic acid, the quencher molecule is separated from the reporter molecule at lower temperatures to unquench a signal from the reporter molecule, and the quencher molecule is in close proximity to the reporter molecule at higher temperatures to quench a signal from the reporter molecule.

In a specific embodiment, in the presence of a target nucleic acid, (i) at lower temperatures, the oligonucleotide is hybridized to the target nucleic acid to form a duplex, and thus the quencher molecule is separated from the reporter molecule to unquench a signal from the reporter molecule, and (ii) at higher temperatures, the first oligonucleotide is not hybridized to the target nucleic acid and exists in a single-stranded state of a hairpin or random coil structure, and thus the quencher molecule is in close proximity to the reporter molecule to quench a signal from the reporter molecule.

In an embodiment, at least one of the n compositions may comprise (a) an oligonucleotide comprising a 5′-tagging portion comprising a nucleotide sequence that is non-complementary to a target nucleic acid sequence and a 3′-targeting portion comprising a hybridizing nucleotide sequence that is complementary to the target nucleic acid sequence, and (b) a label.

In an embodiment, at least one of the n compositions may provide a signal from the label using the formation of a duplex in a manner dependent on hybridization of the oligonucleotide with the target nucleic acid and cleavage of the 5′-tagging portion of the oligonucleotide. For example, the composition may provide a signal from a label using various principles of signal provision by a duplex formed in a manner dependent on the cleavage of the mediation oligonucleotide as described above. In particular, the oligonucleotide may serve as a mediation oligonucleotide to provide a signal from the label. Specifically, the signal is provided in a predetermined temperature range, and the intensity of the signal (e.g., a relative fluorescence unit (RFU) value) is proportional to the amount of amplified target nucleic acids.

In an embodiment, a mediation oligonucleotide (i.e., a first oligonucleotide) hybridized to the target nucleic acid is cleaved to release a fragment (i.e., a 5′-tagging portion), the fragment is specifically hybridized to the capture oligonucleotide and is extended to form an extension strand, resulting in formation of an extended duplex between the extension strand and the capture oligonucleotide, thereby providing a signal indicating the presence of the target nucleic acid at a predetermined temperature.

In an embodiment, where a third oligonucleotide comprising a hybridizing nucleotide sequence complementary to the extended strand is used, the hybridization of the third oligonucleotide with the extended strand forms another type of a duplex providing a signal indicating the presence of the target nucleic acid.

In an embodiment, where a third oligonucleotide comprising a hybridizing nucleotide sequence complementary to the capture oligonucleotide is used, the formation of a duplex between the third oligonucleotide and the capture oligonucleotide is inhibited by the formation of the duplex between the extended strand and the capturing oligonucleotide, thereby providing a signal indicating the presence of the target nucleic acid.

In an embodiment, the label may be interactive dual labels, and the interactive dual labels may be linked to either strand of the duplex.

In the presence of a target nucleic acid, the quencher molecule is separated from the reporter molecule at lower temperatures to unquench a signal from the reporter molecule, and the quencher molecule is in close proximity to the reporter molecule at higher temperatures to quench a signal from the reporter molecule.

In a specific embodiment, the interactive dual labels may be linked to a duplex formed in a manner dependent on hybridization of an oligonucleotide with a target nucleic acid and cleavage of the 5′-tagging portion of the oligonucleotide.

For example, the interactive dual labels may be all linked to one strand of a duplex, and in the presence of a target nucleic acid, (i) at lower temperatures, the duplex may maintain its double-stranded state, and thus the quencher molecule may be separated from the reporter molecule to unquench a signal from the reporter molecule, and (ii) at higher temperatures, the duplex may be denatured into a single-stranded state of a hairpin or random coil structure, and thus the quencher molecule may be in close proximity to the reporter molecule to quench a signal from the reporter molecule.

In an embodiment, at least one of the n compositions may comprise (a) an oligonucleotide comprising a tagging portion comprising a nucleotide sequence that is non-complementary to a target nucleic acid sequence and a targeting portion comprising a hybridizing nucleotide sequence that is complementary to the target nucleic acid sequence, and (b) a label.

In an embodiment, the tagging portion of the oligonucleotide may be in a single-stranded form or a double-stranded form.

In an embodiment, when the tagging portion of the oligonucleotide is in a double-stranded form, the tagging portion in a double-stranded form may be designed such that it maintains its double-stranded form not to provide a signal at lower temperatures but it is dissociated into a single-stranded form to provide a signal at higher temperatures. The temperature range in which a signal is provided may be adjusted according to the Tm value of the tagging portion in a double-stranded form.

In an embodiment, the tagging portion of the oligonucleotide may be in a double-stranded form by intramolecular or intermolecular hybridization.

In an embodiment, when the tagging portion of the oligonucleotide is in the double-stranded form due to intramolecular hybridization, the tagging portion of the oligonucleotide may be designed to form a hairpin structure (i.e., a stem-loop structure) at lower temperatures. In this case, the tagging portion in a double-stranded form may include a partial single-stranded form (for example, a loop portion).

In an embodiment, when the tagging portion of the oligonucleotide is in a double-stranded form by intermolecular hybridization, the tagging portion of the oligonucleotide may be designed to be in a double-stranded form with a tagging-hybridizing oligonucleotide comprising a hybridizing nucleotide sequence complementary to the tagging portion at lower temperatures.

As used herein, the term “tagging-hybridizing oligonucleotide” refers to an oligonucleotide that hybridizes with a tagging portion in a single-stranded form to form a tagging portion in a double-stranded form by intermolecular hybridization. The tagging-hybridizing oligonucleotide may be designed to hybridize with the tagging portion at lower temperatures to form a tagging portion in a double-stranded form, but to dissociate into a single-stranded form at higher temperatures.

In an embodiment, the composition may provide a signal from the label dependent on hybridization of the oligonucleotide with a target nucleic acid, cleavage of the tagging portion of the oligonucleotide in a double-stranded form, and dissociation of the cleaved tagging portion into a single-stranded form. Specifically, the signal may be provided in a high temperature range, for example, a temperature range higher than the Tm of the tagging portion.

In an embodiment, the label may be interactive triple labels comprising one reporter molecule and two quencher molecules.

In an embodiment, in the presence of a target nucleic acid, the interactive triple labels may be configured such that at least one of the two quenchers is in close proximity to the reporter molecule at lower temperatures to quench a signal from the reporter molecule, and all of the two quencher molecules are separated from the reporter molecule at higher temperatures to unquench a signal from the reporter molecule.

In an embodiment, one quencher molecule of the interactive triple labels may be linked to a targeting portion of the oligonucleotide, and the other quencher molecule and the reporter molecule may be linked to a site such that when the tagging portion of the oligonucleotide is cleaved and released, they can be released together with the cleaved tagging portion. In particular, the reporter molecule may be linked to a site such that it is capable of reversibly interacting with the quencher molecule linked to the targeting portion.

According to a specific embodiment, one reporter molecule and one quencher molecule of the interactive triple labels may be linked to the tagging portion of the oligonucleotide in a double-stranded form, and the other quencher molecule may be linked to the targeting portion of the oligonucleotide. In particular, the reporter molecule and the quencher molecule linked to the tagging portion may be linked to a site such that they are capable of reversibly interacting with each other according to the form of the tagging portion (double-stranded form or single-stranded form).

For example, in the presence of a target nucleic acid, one reporter molecule and one quencher molecule of the interactive triple labels may be separated from the other quencher molecule by cleavage of the tagging portion of the oligonucleotide in a double-stranded form, (i) the cleaved tagging portion may maintain its double-stranded form at lower temperatures such that the reporter molecule and the quencher molecule linked to the tagging portion are in close proximity to each other, allowing the quencher molecule to quench a signal from the reporter molecule, and (ii) the cleaved tagging portion in a double-stranded form may dissociate into a single-stranded form at higher temperatures such that the reporter molecule and the quencher molecule are separated from each other, allowing the quencher molecule to unquench a signal from the reporter molecule.

In an embodiment, the cleavage of the tagging portion of the oligonucleotide in a double-stranded form may be performed using polymerase having nuclease activity or using an additional nuclease, but is not limited thereto. The nuclease may be a naturally occurring, unmodified, or modified nuclease.

In an embodiment, the composition may provide a signal by using formation of a duplex in a manner dependent on the hybridization of the oligonucleotide with a target nucleic acid and cleavage of the tagging portion of the oligonucleotide. For example, the composition may provide a signal using various principles of signal provision by a duplex formed in a manner dependent on cleavage of the mediation oligonucleotide as described above. In particular, the oligonucleotide may serve as a mediation oligonucleotide to provide a signal. Specifically, the signal may be provided at a high detection temperature.

In an embodiment, the label may be interactive triple labels comprising one reporter molecule and two quencher molecules.

In an embodiment, in the presence of a target nucleic acid, the interactive triple labels may be configured such that at least one of the two quenchers is in close proximity to the reporter molecule at lower temperatures to quench a signal from the reporter molecule, but all the two quencher molecules are separated from the reporter molecule at higher temperatures to unquench a signal from the reporter molecule.

In an embodiment, one quencher molecule and one reporter molecule of the interactive triple labels may be linked to the oligonucleotide, and the other quencher molecule may be linked to a capture oligonucleotide.

In an embodiment, one quencher molecule and one reporter molecule of the interactive triple labels may be linked to the oligonucleotide, and the other quencher molecule may be linked to a capture oligonucleotide. In particular, when the tagging portion of the oligonucleotide is cleaved and released, the reporter molecule may be released together with the cleaved tagging portion, and may be linked to a site such that it is capable of being separated from the quencher molecule linked to the oligonucleotide.

According to a specific embodiment, one quencher molecule of the interactive triple labels may be linked to the targeting portion of the oligonucleotide, the reporter molecule may be linked to the tagging portion of the oligonucleotide, and the other quencher molecule may be linked to the capture oligonucleotide. In particular, the linking of the reporter molecule to the tagging portion of the oligonucleotide and the linking of the quencher molecule to the capture oligonucleotide may be accomplished at sites such that they are capable of reversibly interacting with each other according to the formation of a duplex (i.e., an extended duplex).

For example, in the presence of a target nucleic acid, the oligonucleotide (i.e., a mediation oligonucleotide) to which one of the two quencher molecules and the reporter molecule are linked and which is hybridized to the target nucleic acid is cleaved to release a fragment (i.e., a tagging portion) to which the reporter molecule is linked, and the fragment to which the reporter molecule is linked is specifically hybridized with a capture oligonucleotide to which the quencher molecule is linked, and the fragment is extended to form an extended strand, resulting in formation of an extended duplex between the extension strand and the capture oligonucleotide.

The resulting extended duplex may (i) maintain a duplex form such that a reporter molecule linked to a fragment (i.e., a tagging portion) released from an oligonucleotide and a quencher molecule linked to a capture oligonucleotide are in close proximity to each other at lower temperatures, allowing the quencher molecule to quench a signal from the reporter molecule, and (ii) dissociate into a single-stranded form such that the reporter molecule and the quencher molecule are separated from each other at higher temperatures, allowing the quencher molecule to unquench a signal from the reporter molecule.

Details of the interactive triple labels as describe above can be found in U.S. Patent Application Publication No. 2019-0024155.

In an embodiment, the label may be interactive dual labels comprising one reporter molecule and one quencher molecule.

In an embodiment, in the presence of a target nucleic acid, the interactive dual labels is configured such that the quencher molecule is in close proximity to the reporter molecule at lower temperatures to quench a signal from the reporter molecule, but the quencher molecule is separated from the reporter molecule at higher temperatures to unquench a signal from the reporter molecule, wherein the unquenching is inversely proportional to an amount of the amplified target nucleic acids.

In an embodiment, one of the interactive dual labels (e.g., a reporter molecule or a quencher molecule) may be linked to an oligonucleotide, and the other (e.g., a quencher molecule or a reporter molecule) may be linked to a capture oligonucleotide. In particular, when the tagging portion of the oligonucleotide is cleaved and released, the linking of a label (e.g., a reporter molecule or a quencher molecule) to the oligonucleotide may be accomplished at a position such that the label can be released together with the cleaved tagging portion.

According to a specific embodiment, one of the interactive dual labels may be linked to the tagging portion of the oligonucleotide, and the other may be linked to a capture oligonucleotide. In particular, the linking of a label (e.g., a reporter molecule or a quencher molecule) to the tagging portion of the oligonucleotide and the linking of a label (e.g., a quencher molecule or a reporter molecule) to the capture oligonucleotide may be accomplished at a position such that they can reversibly interact with each other according to the formation of a duplex (i.e., an extended duplex).

For example, in the presence of a target nucleic acid, an oligonucleotide (i.e., mediation oligonucleotide) to which a quencher molecule (or reporter molecule) is linked and which is hybridized to the target nucleic acid, is cleaved to release a fragment (i.e., tagging portion) to which the quencher molecule (or reporter molecule) is linked, and the fragment to which the quencher molecule (or reporter molecule) is linked is specifically hybridized to a capture oligonucleotide to which the reporter molecule (or quencher molecule) is linked, and the fragment is extended to form an extended strand, resulting in formation of an extended duplex between the extended strand and the capture oligonucleotide.

In an embodiment, the non-cleaved tagging portion of the oligonucleotide and the capture oligonucleotide hybridize to each other to form a duplex at a lower temperature, the duplex dissociates but the extended duplex maintains its duplex form at a middle temperature, and both the duplex and the extended duplex dissociates at a higher temperature.

In an embodiment, the formed extended duplex may maintain its double-stranded form at the lower temperature and the higher temperature to bring a quencher molecule (or a reporter molecule) linked to a fragment (i.e., a tagging portion) released from the oligonucleotide and a reporter molecule (or a quencher molecule) linked to a capture oligonucleotide into proximity to each other, allowing the quencher molecule to quench a signal from the reporter molecule.

On the other hand, the duplex between the non-cleaved tagging portion of a second oligonucleotide and the capture oligonucleotide may (i) maintain its double-stranded form at a lower temperature to bring a quencher molecule (or a reporter molecule) linked to the oligonucleotide and a reporter molecule (or a quencher molecule) linked to the capture oligonucleotide into proximity to each other, allowing the quencher molecule to quench a signal from the reporter molecule, but (ii) dissociate into a single-stranded form at a higher temperature to separate the reporter molecule from the quencher molecule, allowing the quencher molecule to unquench the signal from the reporter molecule.

In an embodiment, in the presence of a target nucleic acid, as the target nucleic acid is amplified, the amount of duplex between the non-cleaved tagging portion of the oligonucleotide and the capture oligonucleotide may decrease, whereas the amount of the extended duplex may increase. Thus, the intensity of the signal (e.g., the RFU value) is inversely proportional to the amount of amplified target nucleic acids.

In an embodiment, at least one of the n compositions comprises (i) a primer; (ii) a Probing and Tagging Oligonucleotide (PTO); (iii) a Capturing and Templating Oligonucleotide (CTO); and (iv) a Labeled Portion Hybridizing Oligonucleotide (LPHO) comprising a nucleotide sequence that hybridizes to a labeled portion to which a reporter molecule and quencher molecule of the CTO are linked.

The above-described composition for target nucleic acid detection provides a signal indicating the presence of a target nucleic acid by a method comprising the steps of:
(a) hybridizing a primer and a Probing and Tagging Oligonucleotide (PTO) with the target nucleic acid;
wherein the primer comprises a hybridizing nucleotide sequence with a first portion of the target nucleic acid,
wherein the PTO comprises in a 5′ to 3′ direction: (i) a 5′-tagging portion comprising a nucleotide sequence not hybridizable with the target nucleic acid, and (ii) a 3′-targeting portion comprising a nucleotide sequence hybridizable with the target nucleic acid with a second region of the target nucleic acid,
wherein the 5′-tagging portion is not hybridized with the target nucleic acid, and the 3′-targeting portion is hybridized with the target nucleic acid,
wherein the primer is located upstream of the PTO;
(b) contacting the resultant of the step (a) to an enzyme having 5′ nuclease activity under conditions for cleavage of the PTO,
wherein the primer is extended to induce cleavage of the PTO by the enzyme having 5′ nuclease activity such that the cleavage releases a fragment comprising the 5′-tagging-portion of the PTO or a portion of the 5′-tagging-portion of the PTO;
(c) hybridizing the fragment released from the PTO with a Capturing and Templating Oligonucleotide (CTO),
wherein the CTO comprises in a 3′ to 5′ direction: (i) a capturing portion comprising a hybridizing nucleotide sequence with the 5′-tagging-portion of the PTO or a portion of the 5′-tagging-portion of the PTO, and (ii) a templating portion comprising a non-hybridizing nucleotide sequence with the 5′-tagging portion and the 3′-targeting portion of the PTO,
wherein the CTO comprises a reporter molecule and a quencher molecule linked thereto,
wherein the released fragment is hybridized with the capturing portion of the CTO;
(d) performing an extension reaction using the resultant of the step (c) and a template-dependent nucleic acid polymerase,
wherein the step (d) is performed in the presence of a Labeled Portion Hybridizing Oligonucleotide (LPHO), wherein the LPHO comprises a hybridizing nucleotide sequence with the labeled portion of the CTO to which a reporter molecule and a quencher molecule is linked,
wherein when the target nucleic acid is present in the sample, the fragment hybridized with the capturing portion of the CTO is extended to generate an extended strand complementary to the templating portion of the CTO, thereby generating an extended duplex between the extended strand and the CTO, wherein the generation of the extended duplex prevents the formation of a hybrid between the CTO and the LPHO,
wherein when the target nucleic acid is not present in the sample, the extended strand is not generated, and instead the hybrid between the CTO and the LPHO is formed; and
(e) detecting the presence of the extended duplex,
wherein the presence of the extended duplex is detected by measuring (i) a signal provided from the extended duplex, (ii) a signal provided from the hybrid between the CTO and the LPHO, or (iii) a signal provide from the extended duplex and the hybrid between the CTO and the LPHO,
wherein the signal is detected at a temperature selected from a temperature range in which all or a portion of the hybrid is present in a dissociated form and all or a portion of the extended duplex is present in a hybridized form, and the presence of the extended strand indicates the presence of the target nucleic acid.

In the case of the composition, when the CTO is single-stranded, a reporter molecule and a quencher molecule on the CTO may be structurally in proximity to each other so that the quencher molecule quenches a signal from the reporter molecule, and when the CTO is hybridized with LPHO to form an CTO/LPHO hybrid, or when the CTO forms an extended duplex (a hybrid of CTO/extended strand) in a target-dependent manner, a reporter molecule and a quencher molecule on the CTO may be structurally spaced apart from each other so that the quencher molecule unquenches a signal from the reporter molecule.

The CTO/LPHO hybrid has a Tm value that is adjustable by the sequence and/or length of LPHO, and the extended duplex has a Tm value that is adjustable by (i) the sequence and/or length of the fragment, (ii) the sequence and/or length of the CTO, or (iii) the sequence and/or length of the fragment and the sequence and/or length of the CTO. Based on this, the Tm value of the CTO/LPHO hybrid and the Tm value of the duplex between CTO/extension strand (i.e., extended duplex) can be predetermined to be different from each other, and due to these two different Tm values, the composition according to the present disclosure has one signal-changing temperature range in which the signal changes depending on the presence of the target nucleic acid and two signal-constant temperature ranges in which the signal is constant even in the presence of the target nucleic acid, in the amplification reaction of the target nucleic acid.

Details of the method of using the LPHO can be found in PCT application No. PCT/KR2024/002516, which is incorporated herein by reference.

### Temperature for Signal Provision

The composition according to the present disclosure is not intended to provide a signal at any temperature in the presence of a target nucleic acid.

As used herein, each of the n compositions has a temperature range in which a signal changes depending on the presence of a target nucleic acid, that is, a signal-changing temperature range (SChTR), and a temperature range in which the signal does not change even in the presence of the target nucleic acid, that is, a signal-constant temperature range (SCoTR), in a reaction (e.g., amplification reaction) with the target nucleic acid.

International Patent Application Publication WO2022-265463 discloses that various signal generation mechanisms for detecting a target nucleic acid have a temperature range (i.e., a signal-changing temperature range) in which a signal changes depending on the presence of the target nucleic acid and a temperature range (i.e., a signal-constant temperature range) in which the signal does not change even in the presence of the target nucleic acid. The publication discloses that various signal generation mechanisms may be classified into (i) an UnderSC signal generation mechanism in which the signal-changing temperature range is lower than the signal-constant temperature range, (ii) a OverSC signal generation mechanism in which the signal-changing temperature range is higher than the signal-constant temperature range, and (iii) an InterSC signal generation mechanism in which the signal-changing temperature range is higher than one of two signal-constant temperature ranges and is lower than the other signal-constant temperature range, according to the number and order of the signal-changing temperature range and the signal-constant temperature range. Based on the findings, a novel method for detecting a plurality of target nucleic acids using a single type of label and a single type of detector in one reaction vessel by various combinations of the three signal generation mechanisms has been proposed.

The method for detecting target nucleic acids as described in WO2022-265463 uses n different compositions corresponding to each of target nucleic acids to detect n different target nucleic acids using a single label in one reaction vessel, wherein each of the n different compositions provides a signal by any one of the UnderSC, OverSC, and InterSC signal generation mechanisms as described above, and adjustment of the signal-changing temperature ranges (e.g., such that they do not overlap with each other) and measurement of a signal change at n temperatures (i.e., detection temperatures) enables the detection of n target nucleic acids. The l^{th} composition for detecting the l^{th} target nucleic acid among the n compositions provides a signal change at the l^{th} detection temperature among the n detection temperatures in the presence of the l^{th} target nucleic acid, and provides a constant signal at other detection temperatures. The i represents an integer from 1 to n, and the l^{th} detection temperature is lower than the (i+1)th detection temperature. The presence of the l^{th} target nucleic acid may be determined by a signal change detected at the l^{th} detection temperature (i.e., the l^{th} signal). In an embodiment, when i is n, there is no (i+1)^{th} detection temperature (i.e., n+1 detection temperature).

The composition according to the present disclosure may be employed as any one of the three compositions described in WO2022-265463.

As used herein, the term “signal-constant temperature range” refers to a temperature range in which a composition for detecting a target nucleic acid provides a constant signal despite the presence of the target nucleic acid. The signal-constant temperature range is a temperature range in which the composition provides a constant signal over reaction time, which may also be referred to herein as a temperature range in which the composition does not provide a significant signal, or a temperature range in which the composition does not provide a signal indicative of the presence of a target nucleic acid.

The term “constant signal” as used herein refers to no substantial change in signal during a reaction between a target nucleic acid and a composition (e.g., during a target nucleic acid amplification reaction). That is, the term refers to all or any signal pattern other than significant signal changes brought about by the amplification of the target nucleic acid present. In particular, the constant signal means no signal change. For example, if the signal during an amplification reaction does not exceed the background signal intensity or the intensity of a signal in the absence of the target nucleic acid, it may be expressed as “the signal is constant”. In the present disclosure, the constant signal may be used interchangeably with the signal that does not change, or the signal that does not show change.

As used herein, the term “signal-changing temperature range” refers to a temperature range in which a composition provides a signal that changes dependent on the presence of a target nucleic acid. The signal-changing temperature range is a temperature range in which the composition provides a signal that changes over reaction time, which may also be referred to herein as a temperature range in which the composition provides a significant signal, or a temperature range in which the composition provides a signal indicative of the presence of the target nucleic acid.

In the present disclosure, “signal change” and/or “constant signal” is based on signals detected at the same temperature during a nucleic acid amplification reaction using the same composition. For example, “signal change” and/or “constant signal” are designated based on difference between signal values detected at the same temperature using n compositions, and more specifically, “signal change” and/or “constant signal” are designated based on (i) a difference between signal values detected at the same temperature in a plurality of cycles, or (ii) a difference between “a reference signal value” described below and a signal value detected at the same temperature as the temperature for which the reference signal value is set. That is, “signal change” and/or “constant signal” is not intended to be designated based on a difference between signal values detected at different temperatures.

Thus, each of the n compositions herein provides a signal in a predetermined temperature range, but not in other temperature ranges.

As described above, each of the n compositions herein may be any one of (i) an UnderSC composition in which the signal-changing temperature range is lower than the signal-constant temperature range; (ii) an OverSC composition in which the signal-changing temperature range is higher than the signal-constant temperature range; and (iii) an InterSC composition in which the signal-changing temperature range is higher than one of two signal-constant temperature ranges and is lower than the other of the two signal-constant temperature ranges.

In one embodiment, the expression “one temperature range is lower than another temperature range” as used in the context of the signal-changing temperature range and signal-constant temperature range for the composition means that the highest temperature of one temperature range is lower than the lowest temperature of another temperature range. Conversely, the expression “one temperature range is higher than another temperature range” means that the lowest temperature of one temperature range is higher than the highest temperature of another temperature range. For example, the expression “a signal-constant temperature range is higher than a signal-changing temperature range” means that the lowest temperature within the signal-constant temperature range is higher than the highest temperature within the signal-changing temperature range.

In one embodiment, the signal-changing temperature range for the composition may be determined depending on the length and/or sequence of the duplex providing a signal change.

In one embodiment, the composition provides a single-typed duplex, the composition may have one signal-changing temperature range and one signal-constant temperature range. The signal-changing temperature range and signal-constant temperature range may be determined depending on the length and/or sequence of the single-typed duplex.

In one embodiment, when the composition provides plural-typed duplexes, in particular, two types of duplexes, the composition may have one signal-changing temperature range and two signal-constant temperature ranges. The signal-changing temperature range and the signal-constant temperature ranges may be determined depending on the lengths and/or sequences of the two types of duplexes.

Details of the UnderSC, OverSC, and InterSC compositions are found in WO2022-265463, which is incorporated herein by reference in its entirety.

### Compositions for Detecting Target Nucleic Acids and Detection Temperatures

In accordance with the methods of the present disclosure, 1 to n−1 of the n compositions are each configured to provide a signal (signal change) indicative of the presence of its corresponding target nucleic acid only at one different detection temperature among the n detection temperatures in the presence of the corresponding target nucleic acid, whereas the other compositions are each configured to provide a signal indicative of the presence of its corresponding target nucleic acid at two to n adjacent detection temperatures among the n detection temperatures arranged in order in the presence of the corresponding target nucleic acid.

According to the methods of the present disclosure, signals are measured at n detection temperatures to detect n target nucleic acids. The n detection temperatures are in an ascending order from a low temperature to a high temperature or in a descending order from a high temperature to a low temperature. For example, where signals are measured at first, second, and third detection temperatures to detect three target nucleic acids, the first detection temperature is the lowest temperature among the three detection temperatures, the second detection temperature is a middle temperature among the three detection temperatures, and the third detection temperature is the highest temperature among the three detection temperatures.

In accordance with the methods of the present disclosure, 1 to n−1 of the n compositions are each configured to provide a signal (signal change) indicative of the presence of its corresponding target nucleic acid only at one different detection temperature among the n detection temperatures in the presence of the corresponding target nucleic acid.

The expression “1 to n−1 of the n compositions are each configured to provide a signal (signal change) indicative of the presence of its corresponding target nucleic acid only at one different detection temperature among the n detection temperatures” is intended to mean that each of the compositions provides a signal indicative of the presence of the corresponding target nucleic acid at one detection temperature but some or all of the compositions do not provide a signal together at the same detection temperature.

For example, when the second composition and the third composition are configured to provide a signal at one different detection temperature, the second composition may be configured to provide a signal indicative of the presence of the second target nucleic acid at the first detection temperature, and the third composition may be configured to provide a signal indicative of the presence of the third target nucleic acid at the second detection temperature. However, the two compositions should not provide signals together at the same detection temperature. For example, if the second composition provides a signal indicative of the presence of a second target nucleic acid at the first detection temperature, the third composition should not provide a signal indicative of the presence of a third target nucleic acid at the first detection temperature.

According to the methods of the present disclosure, the other compositions are each configured to provide a signal indicative of the presence of its corresponding target nucleic acid at two to n adjacent detection temperatures among the n detection temperatures arranged in order in the presence of the corresponding target nucleic acid.

As used herein, the term “adjacent detection temperatures” refers to detection temperatures which are adjacent to each other among the n detection temperatures arranged in order and which are selected according to the methods of the present disclosure. The adjacent detection temperatures do not include detection temperatures separated from each other in temperature order. For example, among the first, second, and third detection temperatures arranged in order (e.g., the first detection temperature is the highest, the second detection temperature is the next, and the third detection temperature is the lowest), it is to be understood that the first and second detection temperatures correspond to adjacent detection temperatures, the second detection temperature and the third detection temperature also correspond to adjacent detection temperatures, and the first, second, and third detection temperatures also correspond to adjacent detection temperatures, but the first and third detection temperatures do not correspond to adjacent detection temperatures.

As an example, a first composition among the n compositions may provide a signal indicative of the presence of a first target nucleic acid at first and second detection temperatures, i.e., two adjacent detection temperatures.

Further, according to the method of the present disclosure, at each of the n detection temperatures, signals are provided by one or two of the n compositions. It is not intended to include that at each of the n detection temperatures, no signal is provided by any of the n compositions or signals are provided by three or more compositions.

According to the methods of the present disclosure, none to n−1 of the n detection temperatures are single-signal detection temperatures selected such that a single signal is provided by one of the n compositions, whereas the other detection temperatures are combined-signal detection temperatures selected such that a combined signal is provided by two of the n compositions.

As used herein, the term “single signal” refers to a signal provided by a reaction between a single target nucleic acid and a composition specific thereto, whereas the term “combined signal” refers to a signal provided by a reaction between two target nucleic acids and two compositions specific thereto. In the present disclosure, a combined signal may be used interchangeably with a mixed signal or a complex signal.

The detection temperature selected or intended such that a single signal is provided from a single target nucleic acid is referred to as a “single-signal detection temperature”, while the detection temperature selected or intended such that a combined signal is provided from two target nucleic acids is referred to as a “combined-signal detection temperature”.

According to the method of the present disclosure, a single signal is provided by one of the n compositions at none to n−1 detection temperatures among the n detection temperatures, and a combined signal is provided by two of the n compositions at the other detection temperature among the n detection temperatures.

In certain embodiments where n is 3, a single signal is provided by one composition at two of the three detection temperatures and a combined signal is provided by two compositions at the other detection temperature.

In certain embodiments where n is 3, a single signal is provided by one composition at one of the three detection temperatures and a combined signal is provided by two compositions at the other two detection temperatures.

In certain embodiments where n is 3, a combined signal is provided by two compositions at all the three detection temperatures.

In certain embodiments where n is 4, a single signal is provided by one composition at three of the four detection temperatures and a combined signal is provided by two compositions at the other detection temperature.

In certain embodiments where n is 4, a single signal is provided by one composition at two of the four detection temperatures and a combined signal is provided by two compositions at the other two detection temperatures.

In certain embodiments where n is 4, a single signal is provided by one composition at one of the four detection temperatures and a combined signal is provided by two compositions at the other three detection temperatures.

In certain embodiments where n is 4, a combined signal is provided by two compositions at all the four detection temperatures.

As described above, the method of the present disclosure can be carried out at the n detection temperatures using various combinations of the n compositions, as long as it meets the requirements: (i) 1 to n−1 of the n compositions are each configured to provide a signal indicative of the presence of its corresponding target nucleic acid only at one different detection temperature among the n detection temperatures in the presence of the corresponding target nucleic acid, whereas the other compositions are each configured to provide a signal indicative of the presence of its corresponding target nucleic acid at two to n adjacent detection temperatures among the n detection temperatures arranged in order in the presence of the corresponding target nucleic acid; and (ii) none to n−1 of the n detection temperatures are single-signal detection temperatures selected such that a single signal is provided by one of the n compositions, whereas the other detection temperatures are combined-signal detection temperatures selected such that a combined signal is provided by two of the n compositions.

Various embodiments of the n compositions that meet the aforementioned requirements can be classified based on (i) the number of compositions that provide a signal indicative of the presence of its corresponding target nucleic acid only at one different detection temperature and (ii) the number of compositions that provide a signal indicative of the presence of its corresponding target nucleic acid at two to n adjacent detection temperatures.

In one embodiment, each of x compositions provides a signal indicative of the presence of its corresponding target nucleic acid only at one different detection temperature, and each of the y compositions provides a signal indicative of the presence of its corresponding target nucleic acid at two to n adjacent detection temperatures, wherein x is an integer from 1 to n−1 and y equals to n−x.

Combinations of the aforementioned x compositions and y compositions may be designated herein as “Sx-My,” wherein the uppercase letter S refers to a single detection temperature; the lowercase letter x refers to the number of compositions having the characteristic of providing a signal only at said single detection temperature; the uppercase letter M refers to multiple detection temperatures; and the lowercase y refers to the number of compositions having the characteristic of providing a signal at said multiple detection temperatures.

Embodiments of combinations of the n compositions according to the present disclosure will be described in detail. The following embodiments are for illustrative purposes only and are not intended to limit the scope of the present invention.

### (1) If n is 3

If n is 3, the first to third compositions are used to detect the first to third target nucleic acids, and signals are measured at the first to third detection temperatures.

The compositions when n is 3 may be represented by S1-M2 or S2-M1.

### (1-1) S1-M2

In one embodiment, one of three compositions is configured to provide a signal indicative of the presence of its corresponding target nucleic acid only at one detection temperature among three detection temperatures arranged in order, and the other two compositions are each configured to provide a signal indicative of the presence of its corresponding target nucleic acid at two adjacent detection temperatures among the three detection temperatures arranged in order.

In another embodiment, one of three compositions is configured to provide a signal indicative of the presence of its corresponding target nucleic acid only at one detection temperature among three detection temperatures arranged in order, another composition is configured to provide a signal indicative of the presence of its corresponding target nucleic acid at two adjacent detection temperatures among the three detection temperatures arranged in order, and the other composition is configured to provide a signal indicative of the presence of its corresponding target nucleic acid at the three detection temperatures arranged in order.

### (1-2) S2-M1

In one embodiment, two of three compositions are each configured to provide a signal indicative of the presence of its corresponding target nucleic acid only at one different detection temperature among three detection temperatures arranged in order, and the other one composition is configured to provide a signal indicative of the presence of its corresponding target nucleic acid at two or three adjacent detection temperatures among the three detection temperatures arranged in order.

In certain embodiments, two of three compositions are each configured to provide a signal indicative of the presence of its corresponding target nucleic acid only at one different detection temperature among three detection temperatures arranged in order, and the other one composition is configured to provide a signal indicative of the presence of its corresponding target nucleic acid at two adjacent detection temperatures among the three detection temperatures arranged in order.

In certain embodiments, two of three compositions are each configured to provide a signal indicative of the presence of its corresponding target nucleic acid only at one different detection temperature among three detection temperatures arranged in order, and the other one composition is configured to provide a signal indicative of the presence of its corresponding target nucleic acid at three adjacent detection temperatures among the three detection temperatures arranged in order

### (2) If n is 4

If n is 4, the first to fourth compositions are used to detect the first to fourth target nucleic acids, and signals are measured at the first to fourth detection temperatures.

The compositions when n is 4 may be represented by S1-M3, S2-M2, or S3-M1.

### (2-1) S1-M3

In one embodiment, one of four compositions is configured to provide a signal indicative of the presence of its corresponding target nucleic acid only at one detection temperature among four detection temperatures arranged in order, and the other three compositions are each configured to provide a signal indicative of the presence of its corresponding target nucleic acid at two adjacent detection temperatures among the four detection temperatures arranged in order.

In another embodiment, one of four compositions is configured to provide a signal indicative of the presence of its corresponding target nucleic acid only at one detection temperature among four detection temperatures arranged in order, another two compositions are each configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three adjacent detection temperatures among the four detection temperatures arranged in order.

In another embodiment, one of four compositions is configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other two compositions are each configured to provide a signal indicative of the presence of its corresponding target nucleic acid at three adjacent detection temperatures among the four detection temperatures arranged in order.

### (2-2) S2-M2

In one embodiment, two of four compositions are each configured to provide a signal indicative of the presence of its corresponding target nucleic acid only at one different detection temperature among four detection temperatures arranged in order, and the other two compositions are each configured to provide a signal indicative of the presence of its corresponding target nucleic acid at two or three adjacent detection temperatures among the four detection temperatures arranged in order.

In another embodiment, two of four compositions are each configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three or four adjacent detection temperatures among the four detection temperatures arranged in order.

### (2-3) S3-M1

In one embodiment, three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at two to four adjacent detection temperatures among the four detection temperatures arranged in order.

In certain embodiments, three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at two adjacent detection temperatures among the four detection temperatures arranged in order.

In certain embodiments, three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at three adjacent detection temperatures among the four detection temperatures arranged in order.

In certain embodiments, three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at four adjacent detection temperatures among the four detection temperatures arranged in order.

### (3) If n is 5

When n is 5, the first to fifth compositions are used to detect the first to fifth target nucleic acids, and signals are measured at the first to fifth detection temperatures.

The compositions when n is 5 may be represented by S1-M4, S2-M3, S3-M2, or S4-M1.

### (3-1) S1-M4

In one embodiment, one of five compositions is configured to provide a signal indicative of the presence of its corresponding target nucleic acid only at one detection temperature among five detection temperatures arranged in order, and the other four compositions are each configured to provide a signal indicative of the presence of its corresponding target nucleic acid at two to five adjacent detection temperatures among the five detection temperatures arranged in order.

### (3-2) S2-M3

In one embodiment, two of five compositions are each configured to provide a signal indicative of the presence of its corresponding target nucleic acid only at one different detection temperature among five detection temperatures arranged in order, and the other three compositions are each configured to provide a signal indicative of the presence of its corresponding target nucleic acid at two to five adjacent detection temperatures among the five detection temperatures arranged in order.

### (3-3) S3-M2

In one embodiment, three of five compositions are each configured to provide a signal indicative of the presence of its corresponding target nucleic acid only at one different detection temperature among five detection temperatures arranged in order, and the other two compositions are each configured to provide a signal indicative of the presence of its corresponding target nucleic acid at two to five adjacent detection temperatures among the five detection temperatures arranged in order.

### (3-4) S4-M1

In one embodiment, four of five compositions are each configured to provide a signal indicative of the presence of its corresponding target nucleic acid only at one different detection temperature among five detection temperatures arranged in order, and the other one composition is configured to provide a signal indicative of the presence of its corresponding target nucleic acid at two to five adjacent detection temperatures among the five detection temperatures arranged in order.

Various embodiments of the compositions as described above and signal provision at detection temperatures are illustrated below:

### (i) If n is 3

### Embodiment 1

| | First | Second | Third |
|---|---|---|---|
| | detection | detection | detection |
| | temperature | temperature | temperature |
| First composition | Provided | Provided | Not provided |
| Second composition | Not provided | Provided | Not provided |
| Third composition | Not provided | Not provided | Provided |

Embodiment 1 is an example of S2-M1.

### Embodiment 2

| | First detection | Second detection | Third detection |
|---|---|---|---|
| | temperature | temperature | temperature |
| First composition | Provided | Not provided | Not provided |
| Second composition | Provided | Provided | Not provided |
| Third composition | Not provided | Not provided | Provided |

Embodiment 2 is an example of S2-M1.

### Embodiment 3

| | First detection | Second detection | Third detection |
|---|---|---|---|
| | temperature | temperature | temperature |
| First composition | Provided | Not provided | Not provided |
| Second composition | Not provided | Provided | Provided |
| Third composition | Not provided | Not provided | Provided |

Embodiment 3 is an example of S2-M1.

### Embodiment 4

| | First detection | Second detection | Third detection |
|---|---|---|---|
| | temperature | temperature | temperature |
| First composition | Provided | Not provided | Not provided |
| Second composition | Not provided | Provided | Not provided |
| Third composition | Not provided | Provided | Provided |

Embodiment 4 is an example of S2-M1.

### Embodiment 5

| | First detection | Second detection | Third detection |
|---|---|---|---|
| | temperature | temperature | temperature |
| First composition | Provided | Provided | Provided |
| Second composition | Not provided | Provided | Not provided |
| Third composition | Not provided | Not provided | Provided |

Embodiment 5 is an example of S2-M1.

### Embodiment 6

| | First detection | Second detection | Third detection |
|---|---|---|---|
| | temperature | temperature | temperature |
| First composition | Provided | Provided | Not provided |
| Second composition | Provided | Provided | Not provided |
| Third composition | Not provided | Not provided | Provided |

Embodiment 6 is an example of S1-M2.

### Embodiment 7

| | First detection | Second detection | Third detection |
|---|---|---|---|
| | temperature | temperature | temperature |
| First composition | Provided | Provided | Not provided |
| Second composition | Not provided | Provided | Provided |
| Third composition | Not provided | Not provided | Provided |

Embodiment 7 is an example of S1-M2.

### Embodiment 8

| | First | Second | Third |
|---|---|---|---|
| | detection | detection | detection |
| | temperature | temperature | temperature |
| First composition | Provided | Not provided | Not provided |
| Second composition | Provided | Provided | Provided |
| Third composition | Not provided | Not provided | Provided |

Embodiment 8 is an example of S2-M1.

### Embodiment 9

| | First detection | Second detection | Third detection |
|---|---|---|---|
| | temperature | temperature | temperature |
| First composition | Provided | Not provided | Not provided |
| Second composition | Provided | Provided | Not provided |
| Third composition | Not provided | Provided | Provided |

Embodiment 9 is an example of S1-M2.

### Embodiment 10

| | First detection | Second detection | Third detection |
|---|---|---|---|
| | temperature | temperature | temperature |
| First composition | Provided | Not provided | Not provided |
| Second composition | Not provided | Provided | Provided |
| Third composition | Not provided | Provided | Provided |

Embodiment 10 is an example of S1-M2.

### Embodiment 11

| | First detection | Second detection | Third detection |
|---|---|---|---|
| | temperature | temperature | temperature |
| First composition | Provided | Not provided | Not provided |
| Second composition | Not provided | Provided | Not provided |
| Third composition | Provided | Provided | Provided |

Embodiment 11 is an example of S2-M1.

### Embodiment 12

| | First detection | Second detection | Third detection |
|---|---|---|---|
| | temperature | temperature | temperature |
| First composition | Provided | Provided | Provided |
| Second composition | Provided | Provided | Not provided |
| Third composition | Not provided | Not provided | Provided |

Embodiment 12 is an example of S1-M2.

### Embodiment 13

| | First detection | Second detection | Third detection |
|---|---|---|---|
| | temperature | temperature | temperature |
| First composition | Provided | Provided | Not provided |
| Second composition | Provided | Provided | Provided |
| Third composition | Not provided | Not provided | Provided |

Embodiment 13 is an example of S1-M2.

### Embodiment 14

| | First detection | Second detection | Third detection |
|---|---|---|---|
| | temperature | temperature | temperature |
| First composition | Provided | Not provided | Not provided |
| Second composition | Provided | Provided | Provided |
| Third composition | Not provided | Provided | Provided |

Embodiment 14 is an example of S1-M2.

### Embodiment 15

| | First detection | Second detection | Third detection |
|---|---|---|---|
| | temperature | temperature | temperature |
| First composition | Provided | Not provided | Not provided |
| Second composition | Not provided | Provided | Provided |
| Third composition | Provided | Provided | Provided |

Embodiment 15 is an example of S1-M2.

### (2) If n is 4

### Embodiment 16

| | First | Second | Third | Fourth |
|---|---|---|---|---|
| | detection | detection | detection | detection |
| | temperature | temperature | temperature | temperature |
| First | Provided | Provided | Not | Not provided |
| composition | | | provided | |
| Second | Not | Provided | Not | Not provided |
| composition | provided | | provided | |
| Third | Not | Not provided | Provided | Not provided |
| composition | provided | | | |
| Fourth | Not | Not provided | Not | Provided |
| composition | provided | | provided | |

Embodiment 16 is an example of S3-M1.

### Embodiment 17

| | First | Second | Third | Fourth |
|---|---|---|---|---|
| | detection | detection | detection | detection |
| | temperature | temperature | temperature | temperature |
| First | Provided | Not provided | Not | Not provided |
| composition | | | provided | |
| Second | Provided | Provided | Not | Not provided |
| composition | | | provided | |
| Third | Not | Not provided | Provided | Not provided |
| composition | provided | | | |
| Fourth | Not | Not provided | Not | Provided |
| composition | provided | | provided | |

Embodiment 17 is an example of S3-M1.

### Embodiment 18

| | First | Second | Third | Fourth |
|---|---|---|---|---|
| | detection | detection | detection | detection |
| | temperature | temperature | temperature | temperature |
| First | Provided | Not provided | Not | Not provided |
| composition | | | provided | |
| Second | Not | Provided | Provided | Not provided |
| composition | provided | | | |
| Third | Not | Not provided | Provided | Not provided |
| composition | provided | | | |
| Fourth | Not | Not provided | Not | Provided |
| composition | provided | | provided | |

Embodiment 18 is an example of S3-M1.

### Embodiment 19

| | First | Second | Third | Fourth |
|---|---|---|---|---|
| | detection | detection | detection | detection |
| | temperature | temperature | temperature | temperature |
| First | Provided | Not provided | Not | Not provided |
| composition | | | provided | |
| Second | Not | Provided | Not | Not provided |
| composition | provided | | provided | |
| Third | Not | Provided | Provided | Not provided |
| composition | provided | | | |
| Fourth | Not | Not provided | Not | Provided |
| composition | provided | | provided | |

Embodiment 19 is an example of S3-M1.

### Embodiment 20

| | First | Second | Third | Fourth |
|---|---|---|---|---|
| | detection | detection | detection | detection |
| | temperature | temperature | temperature | temperature |
| First | Provided | Not provided | Not | Not provided |
| composition | | | provided | |
| Second | Not | Provided | Not | Not provided |
| composition | provided | | provided | |
| Third | Not | Not provided | Provided | Provided |
| composition | provided | | | |
| Fourth | Not | Not provided | Not | Provided |
| composition | provided | | provided | |

Embodiment 20 is an example of S3-M1.

### Embodiment 21

| | First | Second | Third | Fourth |
|---|---|---|---|---|
| | detection | detection | detection | detection |
| | temperature | temperature | temperature | temperature |
| First | Provided | Not provided | Not | Not provided |
| composition | | | provided | |
| Second | Not | Provided | Not | Not provided |
| composition | provided | | provided | |
| Third | Not | Not provided | Provided | Not provided |
| composition | provided | | | |
| Fourth | Not | Not provided | Provided | Provided |
| composition | provided | | | |

Embodiment 21 is an example of S3-M1.

### Embodiment 22

| | First | Second | Third | Fourth |
|---|---|---|---|---|
| | detection | detection | detection | detection |
| | temperature | temperature | temperature | temperature |
| First | Provided | Provided | Provided | Not provided |
| composition | | | | |
| Second | Not | Provided | Not | Not provided |
| composition | provided | | provided | |
| Third | Not | Not provided | Provided | Not provided |
| composition | provided | | | |
| Fourth | Not | Not provided | Not | Provided |
| composition | provided | | provided | |

Embodiment 22 is an example of S3-M1.

### Embodiment 23

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Not | Not |
| composition | | | provided | provided |
| Third | Not | Not | Provided | Not |
| composition | provided | provided | | provided |
| Fourth | Not | Not | Not | Provided |
| composition | provided | provided | provided | |

Embodiment 23 is an example of S2-M2.

### Embodiment 24

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Provided | Not |
| composition | provided | | | provided |
| Third | Not | Not | Provided | Not |
| composition | provided | provided | | provided |
| Fourth | Not | Not | Not | Provided |
| composition | provided | provided | provided | |

Embodiment 24 is an example of S2-M2.

### Embodiment 25

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Not | Not |
| composition | provided | | provided | provided |
| Third | Not | Not | Provided | Provided |
| composition | provided | provided | | |
| Fourth | Not | Not | Not | Provided |
| composition | provided | provided | provided | |

Embodiment 25 is an example of S2-M2.

### Embodiment 26

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Not | Not |
| composition | provided | | provided | provided |
| Third | Not | Not | Provided | Not |
| composition | provided | provided | | provided |
| Fourth | Not | Not | Provided | Provided |
| composition | provided | provided | | |

Embodiment 26 is an example of S2-M2.

### Embodiment 27

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Not |
| composition | | | | provided |
| Third | Not | Not | Provided | Not |
| composition | provided | provided | | provided |
| Fourth | Not | Not | Not | Provided |
| composition | provided | provided | provided | |

Embodiment 27 is an example of S3-M1.

### Embodiment 28

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Provided | Provided | Not |
| composition | provided | | | provided |
| Fourth | Not | Not | Not | Provided |
| composition | provided | provided | provided | |

Embodiment 28 is an example of S2-M2.

### Embodiment 29

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Not | Provided | Provided |
| composition | provided | provided | | |
| Fourth | Not | Not | Not | Provided |
| composition | provided | provided | provided | |

Embodiment 29 is an example of S2-M2.

### Embodiment 30

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Not | Provided | Not |
| composition | provided | provided | | provided |
| Fourth | Not | Not | Provided | Provided |
| composition | provided | provided | | |

Embodiment 30 is an example of S2-M2.

### Embodiment 31

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Provided |
| composition | provided | | | |
| Third | Not | Not | Provided | Not |
| composition | provided | provided | | provided |
| Fourth | Not | Not | Not | Provided |
| composition | provided | provided | provided | |

Embodiment 31 is an example of S3-M1.

### Embodiment 32

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Not |
| composition | provided | | | provided |
| Third | Not | Provided | Provided | Not |
| composition | provided | | | provided |
| Fourth | Not | Not | Not | Provided |
| composition | provided | provided | provided | |

Embodiment 32 is an example of S2-M2.

### Embodiment 33

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Not |
| composition | provided | | | provided |
| Third | Not | Not | Provided | Provided |
| composition | provided | provided | | |
| Fourth | Not | Not | Not | Provided |
| composition | provided | provided | provided | |

Embodiment 33 is an example of S2-M2.

### Embodiment 34

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Provided | Provided | Provided | Not |
| composition | | | | provided |
| Fourth | Not | Not | Not | Provided |
| composition | provided | provided | provided | |

Embodiment 34 is an example of S3-M1.

### Embodiment 35

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Provided | Provided | Provided |
| composition | provided | | | |
| Fourth | Not | Not | Not | Provided |
| composition | provided | provided | provided | |

Embodiment 35 is an example of S3-M1.

### Embodiment 36

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Provided | Provided | Not |
| composition | provided | | | provided |
| Fourth | Not | Not | Provided | Provided |
| composition | provided | provided | | |

Embodiment 36 is an example of S2-M2.

### Embodiment 37

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Not | Provided | Provided |
| composition | provided | provided | | |
| Fourth | Not | Not | Provided | Provided |
| composition | provided | provided | | |

Embodiment 37 is an example of S2-M2.

### Embodiment 38

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Not | Provided | Not |
| composition | provided | provided | | provided |
| Fourth | Not | Provided | Provided | Provided |
| composition | provided | | | |

Embodiment 38 is an example of S3-M1.

### Embodiment 39

| | First | Second | Third | Fourth |
|---|---|---|---|---|
| | detection | detection | detection | detection |
| | temperature | temperature | temperature | temperature |
| First | Provided | Provided | Provided | Provided |
| composition | | | | |
| Second | Not | Provided | Not | Not |
| composition | provided | | provided | provided |
| Third | Not | Not | Provided | Not |
| composition | provided | provided | | provided |
| Fourth | Not | Not | Not | Provided |
| composition | provided | provided | provided | |

Embodiment 39 is an example of S3-M1.

### Embodiment 40

| | First | Second | Third | Fourth |
|---|---|---|---|---|
| | detection | detection | detection | detection |
| | temperature | temperature | temperature | temperature |
| First | Provided | Provided | Provided | Not |
| composition | | | | provided |
| Second | Provided | Provided | Not | Not |
| composition | | | provided | provided |
| Third | Not | Not | Provided | Not |
| composition | provided | provided | | provided |
| Fourth | Not | Not | Not | Provided |
| composition | provided | provided | provided | |

Embodiment 40 is an example of S2-M2.

### Embodiment 41

| | First | Second | Third | Fourth |
|---|---|---|---|---|
| | detection | detection | detection | detection |
| | temperature | temperature | temperature | temperature |
| First | Provided | Provided | Provided | Not |
| composition | | | | provided |
| Second | Not | Provided | Not | Not |
| composition | provided | | provided | provided |
| Third | Not | Not | Provided | Provided |
| composition | provided | provided | | |
| Fourth | Not | Not | Not | Provided |
| composition | provided | provided | provided | |

Embodiment 41 is an example of S2-M2.

### Embodiment 42

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Provided | Not |
| composition | | | | provided |
| Third | Not | Not | Provided | Not |
| composition | provided | provided | | provided |
| Fourth | Not | Not | Not | Provided |
| composition | provided | provided | provided | |

Embodiment 42 is an example of S2-M2.

### Embodiment 43

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Not | Not |
| composition | | | provided | provided |
| Third | Not | Not | Provided | Provided |
| composition | provided | provided | | |
| Fourth | Not | Not | Not | Provided |
| composition | provided | provided | provided | |

Embodiment 43 is an example of S1-M3.

### Embodiment 44

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Not | Not |
| composition | | | provided | provided |
| Third | Not | Not | Provided | Not |
| composition | provided | provided | | provided |
| Fourth | Not | Not | Provided | Provided |
| composition | provided | provided | | |

Embodiment 44 is an example of S1-M3.

### Embodiment 45

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Provided | Provided |
| composition | provided | | | |
| Third | Not | Not | Provided | Not |
| composition | provided | provided | | provided |
| Fourth | Not | Not | Not | Provided |
| composition | provided | provided | provided | |

Embodiment 45 is an example of S2-M2.

### Embodiment 46

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Provided | Not |
| composition | provided | | | provided |
| Third | Not | Not | Provided | Provided |
| composition | provided | provided | | |
| Fourth | Not | Not | Not | Provided |
| composition | provided | provided | provided | |

Embodiment 46 is an example of S1-M3.

### Embodiment 47

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Not | Not |
| composition | provided | | provided | provided |
| Third | Not | Not | Provided | Provided |
| composition | provided | provided | | |
| Fourth | Not | Not | Provided | Provided |
| composition | provided | provided | | |

Embodiment 47 is an example of S1-M3.

### Embodiment 48

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Provided |
| composition | | | | |
| Third | Not | Not | Provided | Not |
| composition | provided | provided | | provided |
| Fourth | Not | Not | Not | Provided |
| composition | provided | provided | provided | |

Embodiment 48 is an example of S3-M1.

### Embodiment 49

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Not |
| composition | | | | provided |
| Third | Not | Provided | Provided | Not |
| composition | provided | | | provided |
| Fourth | Not | Not | Not | Provided |
| composition | provided | provided | provided | |

Embodiment 49 is an example of S2-M2.

### Embodiment 50

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Not |
| composition | | | | provided |
| Third | Not | Not | Provided | Provided |
| composition | provided | provided | | |
| Fourth | Not | Not | Not | Provided |
| composition | provided | provided | provided | |

Embodiment 50 is an example of S2-M2.

### Embodiment 51

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Provided | Provided | Provided |
| composition | provided | | | |
| Fourth | Not | Not | Not | Provided |
| composition | provided | provided | provided | |

Embodiment 51 is an example of S2-M2.

### Embodiment 52

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Provided | Provided | Not |
| composition | provided | | | provided |
| Fourth | Not | Not | Provided | Provided |
| composition | provided | provided | | |

Embodiment 52 is an example of S1-M3.

### Embodiment 53

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Not | Provided | Provided |
| composition | provided | provided | | |
| Fourth | Not | Not | Provided | Provided |
| composition | provided | provided | | |

Embodiment 53 is an example of S1-M3.

### Embodiment 54

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Not | Provided | Not |
| composition | provided | provided | | provided |
| Fourth | Not | Provided | Provided | Provided |
| composition | provided | | | |

Embodiment 54 is an example of S2-M2.

### Embodiment 55

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Provided |
| composition | provided | | | |
| Third | Not | Provided | Provided | Not |
| composition | provided | | | provided |
| Fourth | Not | Not | Not | Provided |
| composition | provided | provided | provided | |

Embodiment 55 is an example of S2-M2.

### Embodiment 56

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Not |
| composition | provided | | | provided |
| Third | Provided | Provided | Provided | Not |
| composition | | | | provided |
| Fourth | Not | Not | Not | Provided |
| composition | provided | provided | provided | |

Embodiment 56 is an example of S2-M2.

### Embodiment 57

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Not |
| composition | provided | | | provided |
| Third | Not | Provided | Provided | Provided |
| composition | provided | | | |
| Fourth | Not | Not | Not | Provided |
| composition | provided | provided | provided | |

Embodiment 57 is an example of S2-M2.

### Embodiment 58

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Provided | Provided | Provided | Provided |
| composition | | | | |
| Fourth | Not | Not | Not | Provided |
| composition | provided | provided | provided | |

Embodiment 58 is an example of S3-M1.

### Embodiment 59

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Provided | Provided | Provided | Not |
| composition | | | | provided |
| Fourth | Not | Not | Provided | Provided |
| composition | provided | provided | | |

Embodiment 59 is an example of S2-M2.

### Embodiment 60

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Provided | Provided | Provided |
| composition | provided | | | |
| Fourth | Not | Not | Provided | Provided |
| composition | provided | provided | | |

Embodiment 60 is an example of S2-M2.

### Embodiment 61

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Not | Provided | Provided |
| composition | provided | provided | | |
| Fourth | Not | Provided | Provided | Provided |
| composition | provided | | | |

Embodiment 61 is an example of S2-M2.

### Embodiment 62

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Not | Provided | Not |
| composition | provided | provided | | provided |
| Fourth | Provided | Provided | Provided | Provided |
| composition | | | | |

Embodiment 62 is an example of S3-M1.

### Embodiment 63

| | First | Second | Third | Fourth |
|---|---|---|---|---|
| | detection | detection | detection | detection |
| | temperature | temperature | temperature | temperature |
| First | Provided | Provided | Provided | Provided |
| composition | | | | |
| Second | Provided | Provided | Not | Not |
| composition | | | provided | provided |
| Third | Not | Not | Provided | Not |
| composition | provided | provided | | provided |
| Fourth | Not | Not | Not | Provided |
| composition | provided | provided | provided | |

Embodiment 63 is an example of S2-M2.

### Embodiment 64

| | First | Second | Third | Fourth |
|---|---|---|---|---|
| | detection | detection | detection | detection |
| | temperature | temperature | temperature | temperature |
| First | Provided | Provided | Provided | Not |
| composition | | | | provided |
| Second | Provided | Provided | Not | Not |
| composition | | | provided | provided |
| Third | Not | Not | Provided | Provided |
| composition | provided | provided | | |
| Fourth | Not | Not | Not | Provided |
| composition | provided | provided | provided | |

Embodiment 64 is an example of S1-M3.

### Embodiment 65

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Provided | Provided |
| composition | | | | |
| Third | Not | Not | Provided | Not |
| composition | provided | provided | | provided |
| Fourth | Not | Not | Not | Provided |
| composition | provided | provided | provided | |

Embodiment 65 is an example of S2-M2.

### Embodiment 66

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Provided | Provided | Provided | Not |
| composition | | | | provided |
| Third | Not | Not | Provided | Provided |
| composition | provided | provided | | |
| Fourth | Not | Not | Not | Provided |
| composition | provided | provided | provided | |

Embodiment 66 is an example of S1-M3.

### Embodiment 67

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Provided |
| composition | | | | |
| Third | Not | Provided | Provided | Not |
| composition | provided | | | provided |
| Fourth | Not | Not | Not | Provided |
| composition | provided | provided | provided | |

Embodiment 67 is an example of S2-M2.

### Embodiment 68

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Provided | Not |
| composition | | | | provided |
| Third | Not | Provided | Provided | Provided |
| composition | provided | | | |
| Fourth | Not | Not | Not | Provided |
| composition | provided | provided | provided | |

Embodiment 68 is an example of S2-M2.

### Embodiment 69

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Provided | Provided | Provided |
| composition | provided | | | |
| Fourth | Not | Not | Provided | Provided |
| composition | provided | provided | | |

Embodiment 69 is an example of S1-M3.

### Embodiment 70

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Provided | Provided | Not | Not |
| composition | | | provided | provided |
| Third | Not | Not | Provided | Provided |
| composition | provided | provided | | |
| Fourth | Not | Provided | Provided | Provided |
| composition | provided | | | |

Embodiment 70 is an example of S1-M3.

### Embodiment 71

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Provided |
| composition | provided | | | |
| Third | Provided | Provided | Provided | Not |
| composition | | | | provided |
| Fourth | Not | Not | Not | Provided |
| composition | provided | provided | provided | |

Embodiment 71 is an example of S2-M2.

### Embodiment 72

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Provided | Not |
| composition | provided | | | provided |
| Third | Provided | Provided | Provided | Provided |
| composition | | | | |
| Fourth | Not | Not | Not | Provided |
| composition | provided | provided | provided | |

Embodiment 72 is an example of S2-M2.

### Embodiment 73

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Provided | Provided | Provided | Provided |
| composition | | | | |
| Fourth | Not | Not | Provided | Provided |
| composition | provided | provided | | |

Embodiment 73 is an example of S2-M2.

### Embodiment 74

| | First | Second | Third | Fourth |
|---|---|---|---|---|
| | detection | detection | detection | detection |
| | temperature | temperature | temperature | temperature |
| First | Provided | Not | Not | Not |
| composition | | provided | provided | provided |
| Second | Not | Provided | Not | Not |
| composition | provided | | provided | provided |
| Third | Not | Not | Provided | Provided |
| composition | provided | provided | | |
| Fourth | Provided | Provided | Provided | Provided |
| composition | | | | |

Embodiment 74 is an example of S2-M2.

In an embodiment of the present disclosure, two of the n compositions do not provide a signal indicative of the presence of its corresponding target nucleic acid at two or more identical detection temperatures. For example, the compositions for detecting the first and second target nucleic acids and the detection temperatures may be optimized such that both the composition for detecting the first target nucleic acid and the composition for detecting the second target nucleic acid do not provide signals at the first detection temperature and the second detection temperature.

In an embodiment of the present disclosure, the number of the combined-signal detection temperature is n. According to this embodiment, when n is 3, the number of the combined-signal detection temperature is 3, and the number of the single-signal detection temperature is 0. In addition, when n is 4, the number of the combined-signal detection temperature is 4, and the number of the single-signal detection temperature is 0.

In an embodiment of the present disclosure, the number of the combined-signal detection temperature is n−1. According to this embodiment, when n is 3, the number of the combined-signal detection temperature is 2, and the number of the single-signal detection temperature is 1. In addition, when n is 4, the number of the combined-signal detection temperature is 3, and the number of the single-signal detection temperature is 1.

In an embodiment of the present disclosure, the number of the combined-signal detection temperature is n−2 or less, provided that the number of the combined-signal detection temperature is not 0 or less. According to this embodiment, when n is 3, the number of the combined-signal detection temperature is 1, and the number of the single-signal detection temperature is 2. In addition, when n is 4, the number of the combined-signal detection temperature is 2, and the number of the single-signal detection temperature is 2. Or when n is 4, the number of the combined-signal detection temperature is 1, and the number of the single-signal detection temperature is 3. As the number of combined-signal detection temperature increases, the process of step (c) for determining the presence of the target nucleic acid may be more complicated. Therefore, minimizing the number of combined-signal detection temperature is desirable to simplify the process of step (c).

Although the compositions when the number (n) of target nucleic acids or detection temperatures is 3 or 4 and the provision of signals at the detection temperatures are illustrated above, those skilled in the art can easily conceive of embodiments when n is 5 or more from the disclosure.

One skilled in the art can implement the method of the present disclosure by appropriate design of the n compositions, and appropriate selection of the n detection temperatures. One of ordinary skill in the art may select one of the embodiments of the above-described compositions, and then design the compositions and select the detection temperatures so that the signal provision at each detection temperature for each composition according to the embodiment may be achieved.

For example, when Embodiment 1 of the above-described compositions is selected to detect three target nucleic acids, a signal provision mechanism (e.g., an UnderSC signal provision mechanism) by which a signal is provided in a low temperature range but not in a high temperature range may be applied to the first composition, another signal provision mechanism (e.g., an InterSC signal provision mechanism) by which a signal is not provided in a low temperature range and a high temperature range but in a temperature range therebetween may be applied to the second composition, and another signal provision mechanism (e.g., an OverSC signal provision mechanism) by which a signal is not provided in a low temperature range but in a high temperature range may be applied to the third composition. The first detection temperature (lowest temperature) may then be selected within a temperature range in which signals are provided only by the first composition (by the first target nucleic acid), the second detection temperature (middle temperature) may be selected within a temperature range in which signals are provided by the first and second compositions (by the first and second target nucleic acids), and the third detection temperature (highest temperature) may be selected within a temperature range in which signals are provided only by the third composition (by the third target nucleic acid), in order to meet the requirements of Embodiment 1 of the compositions.

### Step (b): Measuring Signals at n Detection Temperatures

In this step, the signals are measured at the n detection temperatures under the single detection channel.

Herein, the n detection temperatures, a first to n^{th} detection temperatures, are in an ascending order from a low temperature to a high temperature or in a descending order from a high temperature to a low temperature.

According to the present disclosure, none to n−1 of the n detection temperatures are single-signal detection temperatures selected such that a single signal is provided by one of the n compositions, whereas the other detection temperatures are combined-signal detection temperatures selected such that a combined signal is provided by two of the n compositions.

As described in step (a) herein, the method of the present disclosure is configured such that at each of the n detection temperatures, signals are provided by one or two of the n compositions. Thus, the detectable signal at each temperature is either a single signal provided from a reaction of one composition with one target nucleic acid, or a combined signal provided from a reaction of two compositions with two target nucleic acids.

Since the signal measured at the single-signal detection temperature is derived from one target nucleic acid, the signal itself can directly indicate the presence of the one target nucleic acid, while since the signal measured at the combined-signal detection temperature is derived from two target nucleic acids, the signal cannot indicate which of the two target nucleic acids is present.

The detection temperatures herein are chosen in consideration of whether or not the n compositions described in step (a) provide a signal at each detection temperature.

Referring again to Embodiment 1,

| | First | Second | Third |
|---|---|---|---|
| | detection | detection | detection |
| | temperature | temperature | temperature |
| First | Provided | Provided | Not |
| composition | | | provided |
| Second | Not | Provided | Not |
| composition | provided | | provided |
| Third | Not | Not | Provided |
| composition | provided | provided | |

the first composition should provide a signal at the first and second detection temperatures but not at the third detection temperature; the second composition should provide a signal at the second detection temperature but not at the first and third detection temperatures; the third composition should provide a signal at the third detection temperature but not at the first and second detection temperatures; the first detection temperature should be selected such that a signal is provided by the first composition; the second detection temperature should be selected such that a signal is provided by the first and second compositions; the third detection temperature should be selected such that a signal is provided by the third composition.

Based on the fact that there is a temperature range in which signals are provided by each composition (referred to herein as a “signal-changing temperature range”) and a temperature range in which signals are not provided (referred to herein as a “signal-constant temperature range”), the first, second, and third detection temperatures may be selected to satisfy the requirements of Embodiment 1 as described above.

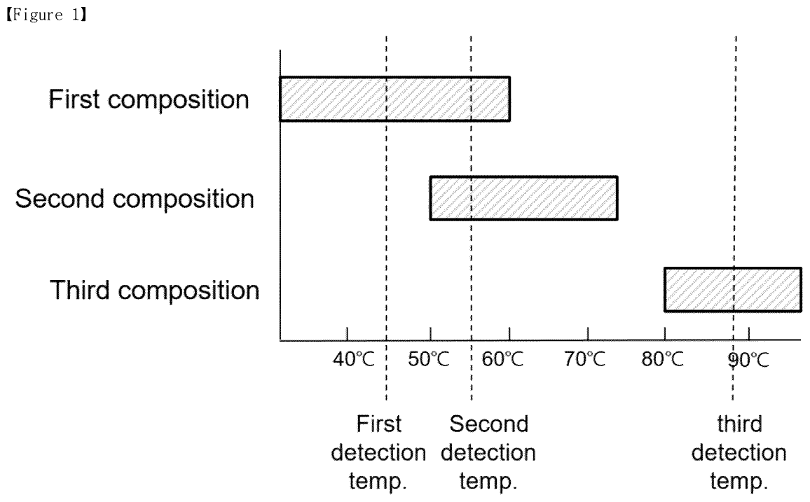

For example, referring to FIG. 1, the first composition may be configured to provide a signal at 60° C. or less (upper bar), the second composition may be configured to provide a signal at 50° C. to 75° C. (middle bar), and the third composition may be configured to provide a signal at 80° C. or more (lower bar).

In this case, the first detection temperature may be selected from a temperature range in which signals can only be provided by the first composition, i.e., a temperature range of less than 50° C. (see the first vertical dotted line from the left), the second detection temperature may be selected from a temperature range in which signals can be provided by the first and second compositions, i.e., a temperature range of 50° C. to 60° C. (see the second vertical dotted line from the left), and the third detection temperature may be selected from a temperature range in which signals can only be provided by the third composition, i.e., a temperature range of 80° C. or more (see the third vertical dotted line from the left).

The n detection temperatures are preferably spaced apart from each other at a predetermined interval to ensure the provision or non-provision of signals by the compositions at each detection temperature. For example, the n detection temperatures may be spaced apart from each other at intervals of at least 2° C., 3° C., 4° C., 5° C., 7° C., 8° C., 9° C., 10° C., 11° C., 12° C., 15° C., or 20° C. However, the interval between each detection temperature does not need to be constant.

In one embodiment where n is 3, the first detection temperature is selected from 50° C. to 70° C., the second detection temperature is selected from 55° C. to 80° C., and the third detection temperature is selected from 60° C. to 90° C.

In certain embodiments where n is 3, the first detection temperature is selected from 55° C., 56° C., 57° C., 58° C., 59° C., 60° C., 61° C., 62° C., 63° C., 64° C., 65° C., and therebetween.

In certain embodiments where n is 3, the second detection temperature is selected from 60° C., 61° C., 62° C., 63° C., 64° C., 65° C., 66° C., 67° C., 68° C., 69° C., 70° C., 71º° C., 72° C., 73° C., 74° C., 75° C., and therebetween.

In certain embodiments where n is 3, the third detection temperature is selected from 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., and therebetween.

In another embodiment where n is 3, the first detection temperature is selected from 60° C. to 90° C., the second detection temperature is selected from 55° C. to 80° C., and the third detection temperature is selected from 50° C. to 70° C.

In one embodiment where n is 4, the first detection temperature is selected from 50° C. to 70° C., the second detection temperature is selected from 55° C. to 75° C., the third detection temperature is selected from 60° C. to 80° C., and the fourth detection temperature is selected from 65° C. to 85° C.

In another embodiment where n is 4, the first detection temperature is selected from 65° C. to 85° C., the second detection temperature is selected from 55° C. to 75° C., the third detection temperature is selected from 60° C. to 80° C., and the fourth detection temperature is selected from 50° C. to 70° C.

The detection temperatures as described above are for illustration only, and those skilled in the art will be able to adjust the detection temperatures appropriately to achieve the best results.

### Step (c): Determination of the Presence of n Target Nucleic Acids

In this step, the presence of a target nucleic acid is determined from the single signal measured at the single-signal detection temperature and the presence of a target nucleic acid from an extracted single signal, which is obtained by extraction from the combined signal measured at the combined-signal detection temperature, whereby the presence of the n target nucleic acids are determined from the signals measured at the n detection temperatures.

The signal which is used to determine the presence of a target nucleic acid includes various signal characteristics obtained from the signal detection, e.g., signal intensity (e.g., RFU (relative fluorescence unit) value or in the case of performing amplification, RFU value at a certain cycle, at a selected cycle, or at end-point], signal change shape (or pattern), Ct value, or values obtained by mathematically processing the characteristics.

In an embodiment of the present disclosure, when an amplification curve is obtained by real-time PCR, various signal values (or characteristics) from the amplification curve may be used to determine the presence of a target nucleic acid.

The term “signal” as used herein includes not only a signal per se obtained at a detection temperature, but also a modified signal obtained by mathematically processing the signal.

In an embodiment, where the mathematical processing is done, the characteristics of the signals should be vulnerable to the mathematical processing. In certain embodiments, the mathematical processing includes calculation (e.g., addition, multiplication, subtraction, and division) using signals or obtaining other values derived from the signals. The signals used to determine the presence of target nucleic acids herein generally are a significant signal. In other words, the signals are those generated being dependent on the presence of a target nucleic acid.

In an embodiment, significance of signals detected may be determined using a threshold value. For example, a threshold value is predetermined from a negative control in consideration of background signals of detector, sensitivity, or label used, and then the significance of signals may be determined by comparing the signals with the threshold value.

According to the methods of the present disclosure, the presence of a target nucleic acid is determined from a single signal measured at a single-signal detection temperature.

Detection of a signal (i.e., a significant signal) at the single-signal detection temperature means that a composition configured to provide a signal at the single-signal detection temperature has reacted with its corresponding target nucleic acid, and accordingly the presence of the target nucleic acid that specifically reacts with the composition is determined. Conversely, no detection of a signal at the single-signal detection temperature means that a composition configured to provide a signal at the single-signal detection temperature has not reacted with its corresponding target nucleic acid, and accordingly the absence of the target nucleic acid that specifically reacts with the composition is determined

For example, referring to Embodiment 1 illustrated herein, the first detection temperature and the third detection temperature are single-signal detection temperatures, and the second detection temperature is a combined-signal detection temperature.

| | First | Second | Third |
|---|---|---|---|
| | detection | detection | detection |
| | temperature | temperature | temperature |
| First | Provided | Provided | Not |
| composition | | | provided |
| Second | Not | Provided | Not |
| composition | provided | | provided |
| Third | Not | Not | Provided |
| composition | provided | provided | |

In Embodiment 1, when a significant signal is detected at the first detection temperature, it is determined that the first target nucleic acid is present, since the signal is provided only by the first composition at the first detection temperature. In addition, when a significant signal is detected at the third detection temperature, it is determined that the third target nucleic acid is present, since the signal is provided only by the third composition at the third detection temperature.

Herein, a signal of no significance may also be expressed as “absence of signal” or “no detection of signal”.

As used herein, the term “by a signal” in connection with determining the presence of a target nucleic acid means that the presence of the target nucleic acid is determined by directly or indirectly using or modifying the measured signal, including using numerical values of the signal or modifications thereof, using the presence/absence of the signal, and comparing the signal to a threshold.

As used herein, the term “determination by a signal” in connection with determination of the presence of a target nucleic acid may include determining the presence of the target nucleic acid in view of the significance of the signal detected at the detection temperature.

According to the methods of the present disclosure, the presence of a target nucleic acid is determined from an extracted single signal, which is obtained by extraction from the combined signal measured at the combined-signal detection temperature.

The presence of the target nucleic acid cannot be determined from the signal itself measured at the combined-signal detection temperature. This is because signals for two target nucleic acids may be detected in an indistinguishable manner at the combined-signal detection temperature.

For example, in Embodiment 1 described above, as undistinguishable signals may be provided by both the first composition and the second composition at the second detection temperature, it is difficult to know whether the measured signal is provided by either or both the first composition and the second composition.

Thus, the method of the present disclosure extracts a single signal from the combined signal measured at the combined-signal detection temperature and uses the extracted single signal to determine the presence of the target nucleic acid.

The extracted single signal may be obtained by extracting only a single signal for one target nucleic acid from the combined signal measured at the combined-signal detection temperature or by eliminating a signal for the other target nucleic acid from the combined signal measured at the combined-signal detecting temperature.

In one embodiment, the extraction of a single signal from the combined signal measured at the combined-signal detection temperature is performed by using (i) the combined signal measured at the combined-signal detection temperature and (ii) a single signal measured at a single-signal detection temperature, which is provided by a composition also providing a signal at the combined signal detection temperature.

For example, referring again to Embodiment 1 as described above, the extraction of a single signal for the second target nucleic acid may be performed by using (i) the combined signal measured at the second detection temperature and (ii) the single signal measured at the first detection temperature, and the extracted single signal may be used to determine the presence of the second target nucleic acid.

The extraction of a single signal from the combined signal may be performed in accordance with a method disclosed in U.S. Patent Application Publication No. 2017-0247750 or 2019-0024155.

The publications teach that there is a certain pattern (rule) of change between signals provided by a target nucleic acid at two detection temperatures in one reaction vessel. Specifically, there is a certain pattern (rule) of signal change between a signal measured at a high detection temperature and a signal measured at a low detection temperature for a target nucleic acid.

In an embodiment, in order to determine whether a signal measured at the combined-signal detection temperature comprises a signal provided by a particular target nucleic acid, a single signal measured at a single-signal detection temperature, which is provided only by the particular target nucleic acid is used.

Specifically, a signal for a target nucleic acid expected to contribute to the combined signal may be extracted by obtaining a difference between a combined signal measured at the combined-signal detection temperature and a single signal provided by another target nucleic acid expected to contribute to the combination signal.

The difference between signals detected at the two detection temperatures may be obtained by a wide variety of approaches.

As used herein, the term “difference” used in relation to “by a difference between signals (or using a difference between signals)” includes a difference obtained by mathematically processing signals itself or modified signals, as well as a difference by the presence or absence of a signal. For example, the difference may be obtained by calculating a ratio or subtraction between signals measured at two detection temperatures. Alternatively, the difference can be obtained by modifying a signal at one detection temperature and comparing it with a signal at another detection temperature. The difference between signals measured at two detection temperatures may be expressed in various aspects. For example, the difference may be expressed as a numerical value, the presence/absence of a signal, or a plot having signal characteristics.

In an embodiment of the present disclosure, the difference between signals measured at two detection temperatures includes a difference obtained by mathematically processing signals measured at two detection temperatures.

As used herein, the term “determined by a difference” includes being determined by the occurrence/non-occurrence of the difference, being determined by the value or range of the difference having a numerical value, and being determined by the plotting result of the difference. Furthermore, “determined by the difference” includes obtaining a value for one target nucleic acid (e.g., CT) based on the difference.

As used herein, the term “by a difference” used in conjunction with determining the presence of a target nucleic acid means that the presence of the target nucleic acid is determined by directly or indirectly using or modifying the difference, including using a numerical value of the difference or a modification thereof, using the presence/absence of a signal, and comparing the difference to a threshold. The terms “by difference” and “by using a difference” may be used interchangeably herein.

The mathematical processing of the signals may be performed by various calculation methods and modifications thereof.

In an embodiment of the present disclosure, the mathematical processing of the signals to obtain the difference between the signals is a calculation of the ratio of the signals measured at two detection temperatures.

As used herein, the term “ratio” means a relationship between two numbers. By using the ratio, the presence of one target nucleic acid can be determined. If the ratio of the signals measured at the two detection temperatures is significant, it becomes an indicator of the presence of one target nucleic acid. For example, if the ratio of the end-point intensities of the signals measured at two detection temperatures is significant (i.e., an increase in the end-point intensity), this indicates the presence of one target nucleic acid.

Extracting a single signal from the combined signal as described above may be performed using a reference value, as disclosed in U.S. Patent Application Publication No. 2017-0247750 or 2019-0024155.

The reference value is a value reflecting a pattern (rule) of signal change exhibited by the target nucleic acid at different detection temperatures. In addition, the reference value is a value reflecting a change in signals provided by the composition at two detection temperatures in the presence of its corresponding target nucleic acid.

For example, when intensities between signals at two detection temperatures provided by a particular composition are different from each other, and the degree of difference between the two signals is calculated by subtraction of the signals, the reference value is a positive value or a negative value other than “0”. As another example, when the degree of difference between the signals at the two detection temperatures is calculated by division of the signals, the reference value is more than 1 or less than 1, excluding “1”.

In certain embodiments, the reference value when the signals at two detection temperatures provided by a particular composition are different from each other may differ by at least 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 12%, 15%, 20%, or 30% compared to the reference value when the two signals are identical.

In one embodiment, when the difference between the signals at the two detection temperatures provided by a particular composition is greater, it is more advantageous to reduce the detection error by using a reference value for the composition.

In an embodiment, a reference value is used to determine the presence of a target nucleic acid that provides a signal at the combined-signal detection temperature but does not provide a signal at the single-signal detection temperature, by using a difference between a signal at the combined-signal detection temperature and a signal at the single-signal detection temperature.

In one embodiment, the extraction of a single signal from the combined signal measured at the combined-signal detection temperature is performed by (i) the difference between the combined signal measured at the combination-signal detection temperature and a single signal provided by another composition at a single-signal detection temperature and (ii) a reference value.

In an embodiment of the present disclosure, a reference value may be used to obtain a difference between signals at the two detection temperatures. For example, a signal at a single-signal detection temperature may be multiplied or divided by a reference value of a target nucleic acid providing the signal at a single-signal detection temperature, and then a difference between the multiplied or divided signal and the signal at the combined-signal detection temperature may be obtained. As another example, the signal at the combined-signal detection temperature may be multiplied or divided by a reference value of a target nucleic acid providing the signal at a single-signal detection temperature, and then a difference between the multiplied or divided signal and the signal at the single-signal detection temperature may be obtained.

According to an implementation of the present disclosure, a reference value is used to determine a threshold. In an embodiment of the present disclosure, a reference value is used as a threshold value with or without modifying the value. The terms “threshold” and “reference value” as used herein may have the same value or meaning in order to determine the presence of the target nucleic acid by analyzing the difference between the signals.

Alternatively, if a reference value is used to obtain the difference between the signal at the single-signal detection temperature and the signal at the combined-signal detection temperature, an additional threshold may be used to determine the significance of the difference, i.e., to determine whether the difference indicates the presence of one target nucleic acid.

In one embodiment of the invention, in order to determine the presence of a target nucleic acid which provides a signal at a combined-signal detection temperature but does not provide a signal at a single-signal detection temperature, the method uses a reference value, which is obtained by (i) reacting a composition with a sample containing its corresponding target nucleic acid in a reaction vessel different from the reaction vessel of step (a) to amplify the target nucleic acid in the sample, and (ii) measuring signals at two detection temperature, and (iii) calculating the difference between the signals.

In an embodiment, the difference between the signals detected at the two detection temperatures obtained in above step (iii) is a value, which is used as a reference value with or without modification.

In an embodiment, a reference value may be obtained by calculating a ratio or subtraction between signals measured at the two detection temperatures. In an embodiment of the present disclosure, a reference value is obtained by calculating a ratio of the signal measured at the combined-signal detection temperature to the signal measured at the single-signal detection temperature. In an embodiment of the present disclosure, a reference value is obtained by calculating a ratio of the signal measured at the single-signal detection temperature to the signal measured at the combined-signal detection temperature.

In an embodiment, a method of calculating a signal difference for a sample and a method of calculating a difference for obtaining a reference value may be identical to or different from each other. For example, the signal difference for the sample may be obtained by subtraction of two signals, and the difference for obtaining the reference value may be obtained by division of two signals. Alternatively, both the signal difference for the sample and the difference for obtaining the reference value may be implemented by dividing the two signals to obtain a ratio.

For a target nucleic acid, a reference value can be obtained under a variety of reaction conditions, including the amount of component (e.g., target nucleic acid, oligonucleotides, enzyme, or dNTPs), pH of buffer, or reaction time. In an embodiment of the present disclosure, the reference value may be obtained under reaction conditions sufficient to provide a saturated signal upon completion of the reaction. In an embodiment of the present disclosure, the difference between signals obtained in the calculation of the reference value has a certain range, and the reference value is selected within the certain range or with reference to the certain range. In an embodiment of the present disclosure, the reference value may be selected as the maximum value or the minimum value of the certain range, or may be selected by referring to the maximum value or the minimum value of the certain range.

In particular, the reference value may be modified in consideration of standard variation, acceptable error ranges, specificity, or sensitivity of the reference values obtained under various conditions.

In an embodiment of the present disclosure, the reference value may be obtained under the same reaction conditions used for the sample, including the components (enzymes or amplification primers, if used), the pH of buffer, and the reaction process. In an embodiment of the present disclosure, the reference value may be obtained using a signal amplification process with or without nucleic acid amplification.

In an embodiment of the present disclosure, when there is a significant difference between the reference value and a difference obtained for determining the presence of a target nucleic acid that provides a signal at the combined-signal detection temperature but does not provide a signal at the single-signal detection temperature, the presence of the target nucleic acid is determined. The reference value may be expressed as a value (e.g., a ratio of the end-point value of the signal intensity) of the same type as the difference obtained to determine the presence of the target nucleic acid.

In a specific example, when the ratio of the end-point value of the signal intensity measured at the single-signal detection temperature to the end-point value of the signal intensity measured at the combined-signal detection temperature is 1.8 and the reference value is 1.1, it may be determined that there is a significant difference between the reference value and a difference obtained to determine the presence of a target nucleic acid providing a signal at the combined-signal detection temperature but not at the single-signal detection temperature. This indicates the presence of a target nucleic acid that provides a signal at the combined-signal detection temperature but does not provide a signal at the single-signal detection temperature.

In an embodiment, when a difference for determining the presence of a target nucleic acid that provides a signal at the combined-signal detection temperature but does not provide a signal at the single-signal detection temperature is equal to or greater than the reference value, the presence of the target nucleic acid is determined.

In an embodiment, when a difference for determining the presence of a target nucleic acid that provides a signal at the combined-signal detection temperature but does not provide a signal at the single-signal detection temperature is equal to or less than the reference value, the presence of the target nucleic acid is determined.

Alternatively, the reference value may be used to calculate the difference between the signals detected at the two detection temperatures. For example, the difference for determining the presence of a target nucleic acid that provides a signal at a combined-signal detection temperature but does not provide a signal at a single-signal detection temperature is calculated by multiplying (or dividing) the signal (e.g., RFU) detected at the single-signal detection temperature by a reference value of another target nucleic acid that provides a signal at the single-signal detection temperature, and then subtracting the result of the multiplication (or division) from the signal (e.g., RFU) detected at the combined-signal detection temperature. When the difference is “0” or greater than (or less than) a predetermined value, it may be determined that the target nucleic acid providing a signal at the combined-signal detection temperature but not providing a signal at the single-signal detection temperature is present.

As another example, a difference for determining the presence of a target nucleic acid that provides a signal at a combined-signal detection temperature but does not provide a signal at a single-signal detection temperature is calculated by multiplying (or dividing) the signal (e.g., RFU) detected at the combined-signal detection temperature by a reference value of another target nucleic acid that provides a signal at the single-signal detection temperature, and then subtracting the result of the multiplication (or division) from the signal (e.g., RFU) detected at the single-signal detection temperature. When the difference is “0” or greater than (or less than) a predetermined value, it may be determined that the target nucleic acid providing a signal at the combined-signal detection temperature but not providing a signal at the single-signal detection temperature is present.

In one embodiment, where a signal is provided in a real-time manner associated with target amplification by PCR, the mathematical processing of the signal involves calculating the ratio of the signal intensity detected at the single-signal detection temperature to the signal intensity detected at the combined-signal detection temperature in each amplification cycle. The calculation results are plotted against cycles and the resulting plot is used to determine the presence of a target nucleic acid that provides a signal at the combined-signal detection temperature but does not provide a signal at the single-signal detection temperature.

In one embodiment, when the signal is provided in a real-time manner associated with target amplification by PCR, a Ct value is a signal for target detection.

A Ct value for a target nucleic acid which provides a signal at the combined-signal detection temperature but does not provide a signal at the single-signal detection temperature may be determined using the signals detected at the two detection temperatures, and for example, may be described as follows: First, a sample to be analyzed is subjected to real-time PCR, and the signals detected at the single-signal detection temperature and the combined-signal detection temperature are obtained, thereby resulting in amplification curves at the two detection temperatures.

(a) When there is no Ct value at a single-signal detection temperature for a target nucleic acid providing a signal at the single-signal detecting temperature, the absence of the target nucleic acid is determined. Then, a Ct value for another target nucleic acid that provides a signal at the combined-signal detection temperature but does not provide a signal at the single-signal detecting temperature is calculated from the amplification curve obtained at the combined-signal detection temperature. If there is no target nucleic acid which provides a signal at the combined-signal detection temperature but does not provide a signal at the single-signal detection temperature, then there is no Ct value for the target nucleic acid.

(b) When there is a Ct value at a single-signal detection temperature for a target nucleic acid providing a signal at the single-signal detection temperature, a ratio of an RFU value obtained at the combined-signal detection temperature to an RFU value obtained at the single-signal detection temperature in a cycle representing the Ct value is calculated. In addition, a ratio of RFU values obtained in a cycle after the cycle representing the Ct value is also calculated. (i) When all the ratios of RFU values are less than a reference value (e.g., a value obtained using only another target nucleic acid providing a signal at the single-signal detection temperature, as described above), the presence of a target nucleic acid providing a signal at the combined-signal detection temperature but not providing a signal at the single-signal detection temperature is determined. Therefore, there is no Ct value for the target nucleic acid. (ii) When all the ratios of RFU values is greater than the reference value, a Ct value calculated from the amplification curve obtained at the combined-signal detecting temperature is determined as a Ct value for a target nucleic acid that provides a signal at the combined-signal detecting temperature but does not provide a signal at a single-signal detecting temperature. (iii) When the ratio of the RFU values in the cycle representing the Ct value is less than the reference value and the ratio of the RFU values after a specific cycle is greater than the reference value, the specific cycle is determined as the Ct value for the target nucleic acid that provides a signal at the combined-signal detection temperature but does not provide a signal at a single-signal detection temperature.

When the calculated ratio is equal to the reference value, the determination may be arbitrarily made. For example, the above-described example specifies that the determination is made in consideration of whether the ratio is less than or equal to a reference value. Further, the determination may be made in consideration of whether the ratio is less than or equal to a reference value or is greater than the reference value.

The Ct value for the target nucleic acid which provides a signal at the combined-signal detection temperature but does not provide a signal at the single-signal detection temperature may alternatively be calculated as follows. The ratio of the RFU value obtained at the combined-signal detection temperature to the RFU value obtained at the single-signal detection temperature in each cycle is calculated; and the Ct value is calculated taking into account a threshold value.

The Ct value for the target nucleic acid which provides a signal at the combined-signal detection temperature but does not provide a signal at the single-signal detection temperature may alternatively be calculated as follows. The RFU values obtained at the single-signal detection temperature in each cycle are modified using the reference value; the ratio of the RFU value obtained at the combined-signal detection temperature to the modified RFU value in each cycle is calculated; and the Ct value is calculated.

In accordance with an embodiment of the present disclosure, the use of a signal detected at the single-signal detection temperature includes obtaining a qualifying value for determining the presence of a target nucleic acid providing the signal at the single-signal detection temperature, and the use of the difference includes obtaining a qualifying value for determining the presence of a target nucleic acid providing the signal at the combined-signal detection temperature but not providing the signal at the single-signal detection temperature.

In accordance with an embodiment of the present disclosure, the use of the difference comprises obtaining a qualifying value for determining the presence of a target nucleic acid that provides a signal at the combined-signal detection temperature but does not provide a signal at a single-signal detection temperature, wherein the qualifying value is obtained by either (i) mathematically processing the signal detected at the single-signal detection temperature and the signal detected at the combined-signal detection temperature, or (ii) if no signal is detected at the single-signal detection temperature, using the signal detected at the combined-signal detection temperature, taking into account that no signal is detected at the single-signal detection temperature.

According to a specific embodiment, the presence of a target nucleic acid that provides a signal at the combined-signal detection temperature but does not provide a signal at the single-signal detection temperature may be determined by (i) multiplying a signal measured at the single-signal detection temperature by a reference value of another target nucleic acid that provides a signal at the single-signal detection temperature (e.g., a ratio of the signal at the single-signal detection temperature to the signal at the combined-signal detection temperature for another target nucleic acid), (ii) subtracting the result of the multiplication from the signal measured at the combined-signal detection temperature, thereby obtaining a signal, i.e., an extracted signal, for the target nucleic acid that provides a signal at the combined-signal detection temperature but does not provide a signal at the single-signal detection temperature, and (iii) using the extracted signal.

The process of determining the presence of three or four target nucleic acids according to each of Embodiments as described above will be illustrated below.

### (1) If n is 3

### Embodiment 1

| | First | Second | Third |
|---|---|---|---|
| | detection | detection | detection |
| | temperature | temperature | temperature |
| First | Provided | Provided | Not |
| composition | | | provided |
| Second | Not | Provided | Not |
| composition | provided | | provided |
| Third | Not | Not | Provided |
| composition | provided | provided | |

For Embodiment 1, the presence of the first target nucleic acid may be determined by a signal measured at the first detection temperature. The presence of the second target nucleic acid may be determined by an extracted signal of the second target nucleic acid, which is extracted from the signal measured at the second detection temperature using the signals measured at the first and second detection temperatures, and optionally a reference value of the first target nucleic acid (e.g., a ratio of signals at the first detection temperature and the second detection temperature provided by the first composition). The presence of the third target nucleic acid may be determined by the signal measured at the third detection temperature.

### Embodiment 2

| | First | Second | Third |
|---|---|---|---|
| | detection | detection | detection |
| | temperature | temperature | temperature |
| First | Provided | Not | Not |
| composition | | provided | provided |
| Second | Provided | Provided | Not |
| composition | | | provided |
| Third | Not | Not | Provided |
| composition | provided | provided | |

For Embodiment 2, the presence of the first target nucleic acid may be determined by an extracted signal of the first target nucleic acid, which is extracted from the signal measured at the first detection temperature, using the signals measured at the first and second detection temperatures, and optionally a reference value of the second target nucleic acid (e.g., a ratio of signals at the first detection temperature and the second detection temperature provided by the second composition). The presence of the second target nucleic acid may be determined by the signal measured at the second detection temperature. The presence of the third target nucleic acid may be determined by the signal measured at the third detection temperature.

### Embodiment 3

| | First | Second | Third |
|---|---|---|---|
| | detection | detection | detection |
| | temperature | temperature | temperature |
| First | Provided | Not | Not |
| composition | | provided | provided |
| Second | Not | Provided | Provided |
| composition | provided | | |
| Third | Not | Not | Provided |
| composition | provided | provided | |

For Embodiment 3, the presence of the first target nucleic acid may be determined by the signal measured at the first detection temperature. The presence of the second target nucleic acid may be determined by the signal measured at the second detection temperature. The presence of the third target nucleic acid may be determined by an extracted signal of the third target nucleic acid, which is extracted from the signal measured at the third detection temperature, using the signals measured at the second and third detection temperatures, and optionally a reference value of the second target nucleic acid (e.g., a ratio of signals at the second detection temperature and the third detection temperature provided by the second composition).

### Embodiment 4

| | First | Second | Third |
|---|---|---|---|
| | detection | detection | detection |
| | temperature | temperature | temperature |
| First | Provided | Not | Not |
| composition | | provided | provided |
| Second | Not | Provided | Not |
| composition | provided | | provided |
| Third | Not | Provided | Provided |
| composition | provided | | |

For Embodiment 4, the presence of the first target nucleic acid may be determined by the signal measured at the first detection temperature. The presence of the second target nucleic acid may be determined by an extracted signal of the second target nucleic acid, which is extracted from the signal measured at the second detection temperature, using the signals measured at the second and third detection temperature, and optionally a reference value of the third target nucleic acid (e.g., a ratio of signals at the second detection temperature and the third detection temperature provided by the third composition). The presence of the third target nucleic acid may be determined by the signal measured at the third detection temperature.

### Embodiment 5

| | First | Second | Third |
|---|---|---|---|
| | detection | detection | detection |
| | temperature | temperature | temperature |
| First | Provided | Provided | Provided |
| composition | | | |
| Second | Not | Provided | Not |
| composition | provided | | provided |
| Third | Not | Not | Provided |
| composition | provided | provided | |

For Embodiment 5, the presence of the first target nucleic acid may be determined by the signal measured at the first detection temperature. The presence of the second target nucleic acid may be determined by an extracted signal of the second target nucleic acid, which is extracted from the signal measured at the second detection temperature, using the signals measured at the first and second detection temperature, and optionally a reference value of the first target nucleic acid (e.g., a ratio of signals at the first detection temperature and the second detection temperature provided by the first composition). The presence of the third target nucleic acid may be determined by an extracted signal of the third target nucleic acid, which is extracted from the signal measured at the third detection temperature, using the signals measured at the first and third detection temperature, and optionally a reference value of the first target nucleic acid (e.g., a ratio of signals at the first detection temperature and the third detection temperature provided by the first composition).

### Embodiment 6

| | First | Second | Third |
|---|---|---|---|
| | detection | detection | detection |
| | temperature | temperature | temperature |
| First | Provided | Provided | Not |
| composition | | | provided |
| Second | Provided | Provided | Not |
| composition | | | provided |
| Third | Not | Not | Provided |
| composition | provided | provided | |

For Embodiment 6, the presence of the first target nucleic acid and the second target nucleic acid may be determined by extracted signals of the first target nucleic acid and the second target nucleic acid, which are extracted from the signal measured the first detection temperature or the second detection temperature, using the signals measured at the first and second detection temperatures, and optionally a reference value of the first target nucleic acid (e.g., a ratio of signals at the first detection temperature and the second detection temperature provided by the first composition) and a reference value of the second target nucleic acid (e.g., a ratio of signals at the first detection temperature and the second detection temperature provided by the second composition) (see U.S. Patent Application Publication No. 2017-0362646). The presence of the third target nucleic acid may be determined by the signal measured at the third detection temperature.

### Embodiment 7

| | First | Second | Third |
|---|---|---|---|
| | detection | detection | detection |
| | temperature | temperature | temperature |
| First composition | Provided | Provided | Not provided |
| Second composition | Not provided | Provided | Provided |
| Third composition | Not provided | Not provided | Provided |

For Embodiment 7, the presence of the first target nucleic acid may be determined by the signal measured at the first detection temperature. The presence of a second target nucleic acid may be determined by an extracted signal of the second target nucleic acid, which is extracted from the signal measured at the second detection temperature, using the signals measured at the first and second detection temperatures, and optionally a reference value of the first target nucleic acid (e.g., a ratio of signals at the first detection temperature and the second detection temperature provided by the first composition). The presence of the third target nucleic acid may be determined by an extracted signal of the third target nucleic acid, which is extracted from the signal measured at a third detection temperature, using the signal measured at a third detection temperature, the extracted signal of the second target nucleic acid, and optionally a reference value of the second target nucleic acid (e.g., a ratio of signals at the second detection temperature and the third detection temperature provided by the second composition).

### Embodiment 8

| | First | Second | Third |
|---|---|---|---|
| | detection | detection | detection |
| | temperature | temperature | temperature |
| First composition | Provided | Not provided | Not provided |
| Second composition | Provided | Provided | Provided |
| Third composition | Not provided | Not provided | Provided |

For Embodiment 8, the presence of the first target nucleic acid may be determined by an extracted signal of the first target nucleic acid, which is extracted from the signal measured at the first detection temperature, using the signals measured at the first and second detection temperatures, and optionally a reference value of a second target nucleic acid (e.g., a ratio of signals at the first detection temperature and the second detection temperature provided by the second composition). The presence of the second target nucleic acid may be determined by the signal measured at the second detection temperature. The presence of the third target nucleic acid may be determined by an extracted signal of the third target nucleic acid, which is extracted from the signal measured at the third detection temperature, using the signals measured at the second and third detection temperatures, and optionally a reference value of the second target nucleic acid (e.g., a ratio of signals at the second detection temperature and the third detection temperature provided by the second composition).

### Embodiment 9

| | First | Second | Third |
|---|---|---|---|
| | detection | detection | detection |
| | temperature | temperature | temperature |
| First composition | Provided | Not provided | Not provided |
| Second composition | Provided | Provided | Not provided |
| Third composition | Not provided | Provided | Provided |

For Embodiment 9, the presence of the second target nucleic acid may be determined by an extracted signal of the second target nucleic acid, which is extracted from the signal measured at the second detection temperature, using the signals measured at the second and third detection temperatures, and optionally a reference value of the third target nucleic acid (e.g., a ratio of signals at the second detection temperature and the third detection temperature provided by the third composition). The presence of the first target nucleic acid may be determined by an extracted signal of the first target nucleic acid, which is extracted from the signal measured at the first detection temperature, using the signals measured at the first and second detection temperatures, and optionally a reference value of the second target nucleic acid (e.g., a ratio of signals at the first detection temperature and the second detection temperature provided by the second composition). The presence of the third target nucleic acid may be determined by the signal measured at the third detection temperature.

### Embodiment 10

| | First | Second | Third |
|---|---|---|---|
| | detection | detection | detection |
| | temperature | temperature | temperature |
| First composition | Provided | Not provided | Not provided |
| Second composition | Not provided | Provided | Provided |
| Third composition | Not provided | Provided | Provided |

For Embodiment 10, the presence of the first target nucleic acid is determined by the signal measured at the first detection temperature. The presence of the second target nucleic acid and the third target nucleic acid is determined by extracted signals of the second target nucleic acid and the third target nucleic acid, which are extracted from the signal measured at the second detection temperature or the signal measured at the third detection temperature, using the signals measured at the second and third detection temperature, and optionally a reference value of the second target nucleic acid (e.g., the ratio of signals at the second detection temperature and the third detection temperature provided by the second composition) and a reference value of the third target nucleic acid (e.g., the ratio of signals at the second detection temperature and the third detection temperature provided by the third composition) (see U.S. Patent Application Publication No. 2017-0362646).

### Embodiment 11

| | First | Second | Third |
|---|---|---|---|
| | detection | detection | detection |
| | temperature | temperature | temperature |
| First composition | Provided | Not provided | Not provided |
| Second composition | Not provided | Provided | Not provided |
| Third composition | Provided | Provided | Provided |

For Embodiment 11, the presence of the first target nucleic acid may be determined by an extracted signal of the first target nucleic acid, which is extracted from the signal measured at the first detection temperature, using the signals measured at the first and third detection temperatures, and optionally a reference value of the third target nucleic acid (e.g., a ratio of signals at the first detection temperature and the third detection temperature provided by the third composition). The presence of the second target nucleic acid may be determined by an extracted signal of the second target nucleic acid, which is extracted from the signal measured at the second detection temperature, using the signals measured at the second and third detection temperatures, and optionally a reference value of the third target nucleic acid (e.g., a ratio of signals at the second detection temperature and the third detection temperature provided by the third composition). The presence of the third target nucleic acid may be determined by the signal measured at the third detection temperature.

### Embodiment 12

| | First | Second | Third |
|---|---|---|---|
| | detection | detection | detection |
| | temperature | temperature | temperature |
| First composition | Provided | Provided | Provided |
| Second composition | Provided | Provided | Not provided |
| Third composition | Not provided | Not provided | Provided |

For Embodiment 12, the presence of the first target nucleic acid and the second target nucleic acid may be determined by extracted signals of the first target nucleic acid and the second target nucleic acid, which are extracted from the signal measured at the first detection temperature or the signal measured at the second detection temperature, using the signals measured at the first and second detection temperatures, and optionally a reference value of the first target nucleic acid (e.g., a ratio of signals at the first detection temperature and the second detection temperature provided by the first composition) and a reference value of the second target nucleic acid (e.g., a ratio of signals at the first detection temperature and the second detection temperature provided by the second composition) (see U.S. Patent Application Publication No. 2017-0362646). The presence of the third target nucleic acid may be determined by an extracted signal of the third target nucleic acid, which is extracted from the signal measured at the third detection temperature, using the signal measured at the third detection temperature, the extracted signal of the first target nucleic acid, and optionally a reference value of the first target nucleic acid (e.g., a ratio of signals at the first detection temperature and the third detection temperature provided by the first composition).

### Embodiment 13

| | First | Second | Third |
|---|---|---|---|
| | detection | detection | detection |
| | temperature | temperature | temperature |
| First composition | Provided | Provided | Not provided |
| Second composition | Provided | Provided | Provided |
| Third composition | Not provided | Not provided | Provided |

For Embodiment 13, the presence of the first target nucleic acid and the second target nucleic acid may be determined by extracted signals of the first target nucleic acid and the second target nucleic acid, which are extracted from the signal measured at the first detection temperature or the signal measured at the second detection temperature, using the signals measured at the first and second detection temperatures, and optionally a reference value of the first target nucleic acid (e.g., a ratio of signals at the first detection temperature and the second detection temperature provided by the first composition) and a reference value of the second target nucleic acid (e.g., a ratio of signals at the first detection temperature and the second detection temperature provided by the second composition) (see U.S. Patent Application Publication No. 2017-0362646). The presence of the third target nucleic acid may be determined by an extracted signal of the third target nucleic acid, which is extracted from the signal measured at the third detection temperature, using the signal measured at the third detection temperature, the extracted signal of the second target nucleic acid, and optionally a reference value of the second target nucleic acid (e.g., a ratio of signals at the first or second detection temperature and the third detection temperature provided by the second composition).

### Embodiment 14

| | First | Second | Third |
|---|---|---|---|
| | detection | detection | detection |
| | temperature | temperature | temperature |
| First composition | Provided | Not provided | Not provided |
| Second composition | Provided | Provided | Provided |
| Third composition | Not provided | Provided | Provided |

For Embodiment 14, the presence of the second target nucleic acid and the third target nucleic acid may be determined by extracted signals of the second target nucleic acid and the third target nucleic acid, which are extracted from the signal measured at the second detection temperature or the signal measured at the third detection temperature, using the signals measured at the second and third detection temperatures, and optionally a reference value of the second target nucleic acid (e.g., a ratio of signals at the second detection temperature and the third detection temperature provided by the second composition) and a reference value of the third target nucleic acid (e.g., a ratio of signals at the second detection temperature and the third detection temperature provided by the third composition) (see U.S. Patent Application Publication No. 2017-0362646). The presence of the first target nucleic acid may be determined by an extracted signal of the first target nucleic acid, which is extracted from the signal measured at the first detection temperature, using the signal measured at the first detection temperature, the extracted signal of the second target nucleic acid, and optionally a reference value of the second target nucleic acid (e.g., a ratio of signals at the second or third detection temperature and the first detection temperature provided by the second composition).

### Embodiment 15

| | First | Second | Third |
|---|---|---|---|
| | detection | detection | detection |
| | temperature | temperature | temperature |
| First composition | Provided | Not provided | Not provided |
| Second composition | Not provided | Provided | Provided |
| Third composition | Provided | Provided | Provided |

For Embodiment 15, the presence of the second target nucleic acid and the third target nucleic acid may be determined by extracted signals of the second target nucleic acid and the third target nucleic acid, which are extracted from the signal measured at the second detection temperature or the signal measured at the third detection temperature, using the signals signal measured at the second and third detection temperatures, and optionally a reference value of the third target nucleic acid (e.g., a ratio of the signal at the second detection temperature and the signal at the third detection temperature provided by the third composition) (see U.S. Patent Application Publication No. 2017-0362646). The presence of the first target nucleic acid may be determined by an extracted signal of the first target nucleic acid, which is extracted from the signal measured at the first detection temperature, using the signal measured at the first detection temperature, the extracted signal of the third target nucleic acid, and optionally a reference value of the third target nucleic acid (e.g., a ratio of signals at the second detection temperature or the third detection temperature and the first detection temperature provided by the third composition).

### (2) If n is 4

Those skilled in the art will be able to readily determine the presence of four target nucleic acids with reference to Embodiments 1-15.

As an example, Embodiment 16 in which n is 4 is described.

### Embodiment 16

| | First | Second | Third | Fourth |
|---|---|---|---|---|
| | detection | detection | detection | detection |
| | temperature | temperature | temperature | temperature |
| First | Provided | Provided | Not | Not |
| composition | | | provided | provided |
| Second | Not | Provided | Not | Not |
| composition | provided | | provided | provided |
| Third | Not | Not | Provided | Not |
| composition | provided | provided | | provided |
| Fourth | Not | Not | Not | Provided |
| composition | provided | provided | provided | |

For Embodiment 16, the presence of the first target nucleic acid may be determined by the signal measured at the first detection temperature. The presence of the second target nucleic acid may be determined by an extracted signal of the second target nucleic acid, which is extracted from the signal measured at the second detection temperature, using the signals measured at the first and second detection temperatures, and optionally a reference value of the first target nucleic acid (e.g., a ratio of signals at the first detection temperature and the second detection temperature provided by the first composition). The presence of the third target nucleic acid may be determined by the signal measured at the third detection temperature. The presence of the fourth target nucleic acid may be determined by the signal measured at the fourth detection temperature.

### II. Kit for Detecting a Target Nucleic Acids

In another aspect of the invention, there is provided a kit for detecting n target nucleic acids in a sample using n detection temperatures, comprising:
(a) n compositions for detecting the n target nucleic acids,
wherein n is an integer of 3 or more,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid,
wherein at each of the n detection temperatures, signals are provided by one or two of the n compositions,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(b) an instruction describing the method according to any one of embodiments as described above.

Since the kit of the present disclosure is manufactured to implement the method of the present disclosure, the common descriptions between them are omitted in order to avoid undue redundancy leading to the complexity of this specification.

All of the present kits described hereinabove may optionally include the reagents required for performing target amplification PCR reactions (e.g., PCR reactions) such as buffers, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity. The kits may also include reagents necessary for performing positive and negative control reactions. Optimal amounts of reagents to be used in a given reaction can be readily determined by the skilled artisan having the benefit of the current disclosure. The components of the kit may be present in separate containers, or multiple components may be present in a single container.

The instructions for describing or practicing the methods of the present invention may be recorded on a suitable storage medium. For example, the instructions may be printed on a substrate, such as paper and plastic. In other embodiments, the instructions may be present as an electronic storage data file present on a suitable computer readable storage medium such as CD-ROM and diskette. In yet other embodiments, the actual instructions may not be present in the kit, but means for obtaining the instructions from a remote source, e.g. via the internet, are provided. An example of this embodiment is a kit that includes a web address where the instructions can be viewed and/or from which the instructions can be downloaded.

### III. Storage Medium and Device for Detection of Target Nucleic Acids

Since the storage medium, the device and the computer program of the prevent invention described herebelow are intended to perform the present methods in a computer, the common descriptions between them are omitted in order to avoid undue redundancy leading to the complexity of this specification.

In another aspect of the present disclosure, there is provided a computer readable storage medium containing instructions to configure a processor to perform a method for detecting n target nucleic acids in a sample using n detection temperatures, the method comprising:
(a) receiving signals measured at the n detection temperatures under a single detection channel,
wherein the signals are obtained by reacting, in a single reaction vessel, a sample suspected of containing at least one of n target nucleic acids with n compositions for detecting n target nucleic acids, and measuring the signals at the n detection temperatures under the single detection channel,
wherein n is an integer of 3 or more,
wherein, during the reacting, the n target nucleic acids in the sample are amplified,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(b) determining the presence of a target nucleic acid from the single signal measured at the single-signal detection temperature and determining the presence of a target nucleic acid from a single signal, which is extracted from the combined signal measured at the combined-signal detection temperature such that the presence of the n target nucleic acids are determined from the signals measured at the n detection temperatures.

In an embodiment, in order to determine the presence of a target nucleic acid that provides a signal at the combined-signal detection temperature but does not provide a signal at the single-signal detection temperature from the combined-signal measured at the combined-signal detection temperature, a reference value of another target nucleic acid that provides a signal at the combined-signal detection temperature and also provides a signal at the single-signal detection temperature is used.

In an embodiment of the present disclosure, a reference value of a target nucleic acid providing a signal at the combined-signal detecting temperature and also providing a signal even at the single-signal detecting temperature is stored in the computer readable storage medium. In an embodiment of the present disclosure, the computer readable storage medium contains instructions to input the reference value in performing the method. According to an embodiment of the present invention, the computer readable storage medium further contains instructions to configure a processor to perform a method for obtaining the reference value.

In another aspect of the present disclosure, there is provided a computer program to be stored on a computer readable storage medium to configure a processor to perform a method for detecting n target nucleic acids in a sample using n detection temperatures, the method comprising:
(a) receiving signals measured at the n detection temperatures under a single detection channel,
wherein the signals are obtained by reacting, in a single reaction vessel, a sample suspected of containing at least one of n target nucleic acids with n compositions for detecting n target nucleic acids, and measuring the signals at the n detection temperatures under the single detection channel,
wherein n is an integer of 3 or more,
wherein, during the reacting, the n target nucleic acids in the sample are amplified,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(b) determining the presence of a target nucleic acid from the single signal measured at the single-signal detection temperature and determining the presence of a target nucleic acid from a single signal, which is extracted from the combined signal measured at the combined-signal detection temperature such that the presence of the n target nucleic acids are determined from the signals measured at the n detection temperatures.

According to an embodiment of the present invention, the computer program contains a reference value of the target nucleic acid sequence providing a signal at the combined-signal detecting temperature and providing a signal even at a single-signal detecting temperature. According to an embodiment of the present invention, the computer program contains instructions to input the reference value in performing the method. According to an embodiment of the present invention, the computer program further contains instructions to configure a processor to perform a method for obtaining the reference value.

The program instructions are operative, when performed by the processor, to cause the processor to perform the present method described above. The program instructions may comprise an instruction to receive signals at n detection temperatures, and an instruction to determine the presence of n target nucleic acid sequences by using the signals received.

The present method described above is implemented in a processor, such as a processor in a stand-alone computer, a network attached computer or a data acquisition device such as a real-time PCR machine.

The types of the computer readable storage medium include various storage medium such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server.

The data (e.g., intensity, amplification cycle number and detection temperature) associated with the signals may be received through several mechanisms. For example, the data may be acquired by a processor resident in a PCR data acquiring device. The data may be provided to the processor in real time as the data is being collected, or it may be stored in a memory unit or buffer and provided to the processor after the experiment has been completed. Similarly, the data set may be provided to a separate system such as a desktop computer system via a network connection (e.g., LAN, VPN, intranet and Internet) or direct connection (e.g., USB or other direct wired or wireless connection) to the acquiring device, or provided on a portable medium such as a CD, DVD, floppy disk, portable HDD or the like to a stand-alone computer system. Similarly, the data set may be provided to a server system via a network connection (e.g., LAN, VPN, intranet, Internet, and wireless communication network) to a client such as a notebook or a desktop computer system.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium such as a portable HDD, USB, floppy disk, CD and DVD. A computer code for implementing the present invention may be implemented in a variety of coding languages such as C, C++, Java, Visual Basic, VBScript, JavaScript, Perl and XML. In addition, a variety of languages and protocols may be used in external and internal storage and transmission of data and commands according to the present invention.

In still another aspect of this invention, there is provided a device for detecting n target nucleic acids in a sample using n detection temperatures, comprising (a) a computer processor and (b) the computer readable storage medium as described above coupled to the computer processor, where n is an integer of 3 or more.

According to an embodiment, the device further comprises a reaction vessel to accommodate the sample and the composition, a temperature controlling means to control temperatures of the reaction vessel and/or a single type detector to detect signals to be generated by the composition.

According to an embodiment, the computer processor permits not only the single type of detector to detect signals provided by a composition for detecting a target nucleic acid at n detection temperatures but also to calculate a difference between signals detected at two of the n detection temperatures. The processor may be prepared in such a manner that a single processor can do two performances: direction of detection of signals at n detection temperatures and calculation of the difference between the signals at the two detection temperatures. Alternatively, the processor unit may be prepared in such a manner that two processors do two performances, respectively.

The first essential feature of the device is to carry the processor to permit the device to detect signals to be provided at n detection temperatures. According to an embodiment, where the signal is generated along with amplification of the target nucleic acid, the device comprises a processor to permit the device to detect signals to be provided at the n detection temperatures at each amplification cycle.

The second essential feature of the device is to carry the processor to process the signal detected at the n detection temperatures to determine the presence of each target nucleic acid.

According to an embodiment, the processor may be embodied by installing software into conventional devices for detection of target nucleic acids (e.g., real-time PCR device). According to an embodiment, the device comprises a processor to permit the device to detect signals at n detection temperatures and to mathematically process the signals at two detection temperatures.

The present invention will now be described in further detail by examples. It would be obvious to those skilled in the art that these examples are intended to be more concretely illustrative and the scope of the present invention as set forth in the appended claims is not limited to or by the examples.

### Example 1: Preparation of Compositions for Detecting Three Target Nucleic Acids and Provision of Signal According to Temperature

<1-1> Preparation of Target Nucleic Acids and Compositions for Detecting them

In order to investigate whether the method according to an embodiment of the present disclosure enables detection of three target nucleic acids, first to third target nucleic acids and first to third compositions for detecting them were prepared.

Genomic DNAs of Chlamydia trachomatis (CT) (Accession No.: ATCC VR-1500, Koramdeolab Co., Ltd.), Neisseria gonorrhoeae (NG) (Accession No.: ATCC 700825, Koramdeolab Co., Ltd.), and Ureasplasma parvum (UP) (Accession No.: ATCC 27815, Koramdeolab Co., Ltd.) as the first to third target nucleic acids were prepared, respectively.

The detection of the first target nucleic acid (CT) was performed according to the PTOCE method (WO2012/096523) which detects a signal by a duplex formed dependent on the presence of a target nucleic acid, and the detection of the second target nucleic acid (NG) and the third target nucleic acid (UP) was performed according to the modified PTOCE method using LPHO (PCT/KR2024/002516).

First, a first composition for detection of the first target nucleic acid (CT) was prepared, which comprises (a) a primer pair consisting of a forward primer (referred to herein as “CT-F”) and a reverse primer (referred to herein as “CT-R”) for amplifying CT; and (b) a first oligonucleotide (referred to herein as “CT-PTO”) and a second oligonucleotide (referred to herein as “CT-CTO”) for providing a signal from CT. Specifically, the CT-PTO was designed to comprise in a 5′ to 3′ direction: (i) a 5′-tagging portion comprising a nucleotide sequence non-complementary to the first to third target nucleic acid sequences, and (ii) a 3′-targeting portion comprising a hybridizing nucleotide sequence complementary to the first target nucleic acid sequence. The CT-CTO was designed to comprise in a 3′ to 5′ direction: (i) a 3′-capturing portion comprising a nucleotide sequence complementary to the 5′-tagging portion or a part of the 5′-tagging portion of the CT-PTO, and (ii) a 5′-templating portion comprising a nucleotide sequence non-complementary to the 5′-tagging portion and the 3′-targeting portion of the CT-PTO. The CT-PTO was blocked at its 3′-terminus with Spacer C3 to prevent extension by DNA polymerase. The CT-CTO was linked with a quencher molecule (BHQ-2) at its 5′-terminus and a reporter molecule (Cal Fluor Red 610) in its 5′-templating portion.

Next, a second composition for detection of the second target nucleic acid (NG) was prepared, which comprises (a) a primer pair consisting of a forward primer (referred to herein as “NG-F”) and a reverse primer (referred to herein as “NG-R”) for amplifying NG; and (b) a first oligonucleotide (referred to herein as “NG-PTO”), a second oligonucleotide (referred to herein as “NG-CTO”), and a third oligonucleotide (referred to herein as “NG-LPHO”) for providing a signal from NG. Specifically, the NG-PTO was designed to comprise in a 5′ to 3′ direction: (i) a 5′-tagging portion comprising a nucleotide sequence non-complementary to the first to third target nucleic acid sequences, and (ii) a 3′-targeting portion comprising a hybridizing nucleotide sequence complementary to the second target nucleic acid sequence. The NG-CTO was designed to comprise in a 3′ to 5′ direction: (i) a 3′-capturing portion comprising a nucleotide sequence complementary to the 5′-tagging portion or a part of the 5′-tagging portion of the NG-PTO, and (ii) a 5′-templating portion comprising a nucleotide sequence non-complementary to the 5′-tagging portion and the 3′-targeting portion of the NG-PTO. The NG-LPHO was designed to have a hybridizing nucleotide sequence complementary to a labeling portion comprising nucleotides to which a reporter molecule and a quencher molecule of the NG-CTO are linked. The NG-PTO, NG-CTO, and NG-LPHO were blocked at their 3′-termini with Spacer C3 to prevent extension by DNA polymerase. The NG-CTO was linked with a quencher molecule (BHQ-2) at its 5′-terminus and a reporter molecule (Cal Fluor Red 610) in its 5′-templating portion.

Next, a third composition for detecting a third target nucleic acid (UP) was prepared, which comprises (a) a primer pair consisting of a forward primer (referred to herein as “UP-F”) and a reverse primer (referred to herein as “UP-R”) for amplifying UP; and a first oligonucleotide (referred to herein as “UP-PTO”), a second oligonucleotide (referred to herein as “UP-CTO”), and a third oligonucleotide (referred to herein as “UP-LPHO”) for providing a signal from UP. Specifically, the UP-PTO was designed to comprise in a 5′ to 3′ direction: (i) a 5′-tagging portion comprising a nucleotide sequence non-complementary to the first to third target nucleic acid sequences, and (ii) a 3′-targeting portion comprising a hybridizing nucleotide sequence complementary to the third target nucleic acid sequence. The UP-CTO was designed to comprise in a 3′ to 5′ direction: (i) a 3′-capturing portion comprising a nucleotide sequence complementary to the 5′-tagging portion or a part of the 5′-tagging portion of the UP-PTO, and (ii) a 5′-templating portion comprising a nucleotide sequence non-complementary to the 5′-tagging portion and the 3′-targeting portion of UP-PTO. The UP-LPHO was designed to have a hybridizing nucleotide sequence complementary to a labeling portion comprising nucleotides to which a reporter molecule and a quencher molecule of the UP-CTO are linked. The UP-PTO, UP-CTO, and UP-LPHO were blocked their 3′-termini with Spacer C3 to prevent extension by DNA polymerase. The UP-CTO was linked with a quencher molecule (BHQ-2) at its 5′-terminus and a reporter molecule (Cal Fluor Red 610) in its 5′-templating portion.

In relation to temperature, the first composition was configured to provide a significant signal indicative of the presence of the first target nucleic acid by allowing formation of an extended duplex dependent on the presence of the first target nucleic acid at 65° C. or lower, but not to provide a significant signal by allowing dissociation of the extended duplex above 70° C.

In the presence of the first target nucleic acid, the first composition allows the CT-PTO to specifically hybridizes to the first target nucleic acid and be then cleaved by DNA polymerase to generate a fragment of the CT-PTO. The fragment is annealed with the capturing portion of the CT-CTO and extended along the templating portion of the CT-CTO as a template to form an extended duplex, thereby providing a signal. Therefore, adjusting the Tm of the extended duplex such that the extended duplex is formed at temperatures of 65° C. or lower can provide a significant signal at 65° C. or lower.

Meanwhile, the second composition was configured to provide a significant signal indicative of the presence of the second target nucleic acid by maintaining the double-stranded extended duplex formed dependent on the presence of the second target nucleic acid at 62° C. to 76° C., but not to provide a significant signal by allowing formation of a double-stranded NG-CTO/NG-LPHO hybrid in addition to the extended duplex below 62° C. and allowing dissociation of both the extended duplex and the NG-CTO/NG-LPHO hybrid above 76° C.

The second composition allows the formation of the NG-CTO/NG-LPHO hybrid, thereby providing a signal, prior to the reaction. During the reaction, the NG-PTO is specifically hybridized with the second target nucleic acid and is then cleaved by DNA polymerase to generate a fragment of the NG-PTO. The fragment is annealed with the capturing portion of the NG-CTO and extended along the templating portion of the NG-CTO as a template to form an extended duplex, thereby providing a signal. The extended duplex competes with the NG-CTO/NG-LPHO hybrid. If the NG-CTO/NG-LPHO hybrid is designed to have a Tm lower than the extended duplex, there will be both the NG-CTO/NG-LPHO hybrid and the extended duplex at lower temperatures; there will be only the extended duplex at middle temperatures; and there will be neither the NG-CTO/NG-LPHO hybrid nor the extended duplex at higher temperatures. Therefore, adjusting the Tm of the NG-CTO/NG-LPHO hybrid and the extended duplex such that the double-stranded extended duplex is maintained at 62° C. to 76° C. can provide a significant signal at 62° C. to 76° C.

In addition, the third composition was configured to provide a significant signal indicative of the presence of the third target nucleic acid by maintaining only the double-stranded extended duplex formed dependent on the presence of the third target nucleic acid at 76° C. or higher, but not to provide a significant signal by maintaining both the double-stranded UP-CTO/UP-LPHO hybrid and the double-stranded extended duplex below 76° C.

Like the second composition, adjusting the Tm of the UP-CTO/UP-LPHO hybrid and the extended duplex such that only the double-stranded extended duplex is maintained at 76° C. or higher can provide a significant signal at 76° C. or higher.

The signals, each indicating the presence of each target nucleic acid, cannot be distinguished by a single detector (single detection channel) in one reaction vessel, because they are provided from a single type of label (i.e., CAL Fluor Red 610).

Oligonucleotides included in the first to third compositions as prepared above are shown in Table 1 below.

TABLE 1
Oligonucleotides in the first,
second, and third compositions

| | | | SEQ |
|---|---|---|---|
| | Oligo | | ID |
| Composition | name | Sequence (5′→3′) | NO: |
| First | CT-F | GAACAAAAAATTCCTCAAAGCTTC | 1 |
| composition | CT-R | TCAAAAAGATCAAGAAGACCAC | 2 |
| | CT- | AACGGTACGACGCGCACGTAGAGA | 3 |
| | PTO | ATATAGCTGGATCTGG[Spacer C3] | |
| | CT- | [BHQ-2]TTATTATTATTTATTATTTA | 4 |
| | CTO | [T(CAL Fluor Red 610)]ATGCG | |
| | | CGTCGTACCGTT[Spacer C3] | |
| Second | NG-F | AAGTCCGCCTATACGCCT | 5 |
| composition | NG-R | CATTTTTGTAATTCAGACCGG | 6 |
| | PTO | ACGGCGCAATACCAGCCGGAAC | 7 |
| | NG- | TGGTTTCATCTGATTAC | |
| | | [Spacer C3] | |
| | CTO | [BHQ-2]TTTTTCTTTTTTGAGCG | 8 |
| | NG- | [T(CAL Fluor Red 610)]CT | |
| | | TCCCTGGTATTGCGCCGT | |
| | | [Spacer C3] | |
| | NG- | CAGGGAAGACGCTCAAAAAAGAAA | 9 |
| | LPHO | AA[Spacer C3] | |
| Third | UP-F | GTTAGATGATAAATTAGGTAATGCTG | 10 |
| composition | UP-R | CAGCTGTATAAATTTTACCTTCAC | 11 |
| | UP- | TGTCGATCGCGTTGGAAATC | 12 |
| | PTO | CACATTTAACAGATAATGTT | |
| | | GAT[Spacer C3] | |
| | UP- | [BHQ-2]TGCGTTCCGTCGCGTAC | 13 |
| | CTO | [T(CAL Fluor Red 610)] | |
| | | GACGTCCAACGCGATCGACA | |
| | | [Spacer C3] | |
| | UP- | GTTGGACGTCAGTACGCGACGG | 14 |
| | LPHO | AACGCA[Spacer C3] | |

BHQ-2: Black Hole Quencher-2

### <1-2> Signal Provision by Each Composition Depending on Temperature

In order to examine the provision of signals by each of the first to third compositions depending on temperature in the presence of its corresponding target nucleic acid, real-time PCR was performed as follows.

First, the first composition was mixed with the first target nucleic acid to prepare a reaction mixture for real-time PCR. Specifically, 4 pmole of CT-F and CT-R (SEQ ID NOs: 1 and 2), 2 pmole of CT-PTO (SEQ ID NO: 3), 1 pmole of CT-CTO (SEQ ID NO: 4), 5 μl of 4× Enzyme Mix (20 U of Taq DNA polymerase), and 5 μL of 4× Buffer Mix (final, 0.8 mM dNTPs, 50 mM KCl, 3.5 mM MgCl₂) (Nanohelix, Korea) was mixed with the first target nucleic acid (Tube 1:1.604 pg of CT genomic DNA) or distilled water (Tube 2; negative control) to prepare a final 20 μL reaction mixture.

Next, the second composition was mixed with the second target nucleic acid to prepare another reaction mixture for real-time PCR. Specifically, 4 pmole of NG-F and NG-R (SEQ ID NOs: 5 and 6), 2 pmole of NG-PTO (SEQ ID NO: 7), 1 pmole of NG-CTO (SEQ ID NO: 8), 4 pmole of NG-LPHO (SEQ ID NO: 9), 5 μL of 4× Enzyme Mix (20 U of Taq DNA polymerase), and 5 μL of 4× Buffer Mix (final, 0.8 mM dNTPs, 50 mM KCl, 3.5 mM MgCl₂) (Nanohelix, Korea) was mixed with the second target nucleic acid (Tube 3:9.498 pg of NG genomic DNA) or distilled water (Tube 4; negative control) to prepare a final 20 μL reaction mixture.

Next, the third composition mixed with the third target nucleic acid to prepare another reaction mixture for real-time PCR. Specifically, 4 pmole of UP-F and UP-R (SEQ ID NOs: 10 and 11), 2 pmole of UP-PTO (SEQ ID NO: 12), 1 pmole of UP-CTO (SEQ ID NO: 13), 4 pmole of UP-LPHO (SEQ ID NO: 14), 5 μL of 4× Enzyme Mix (20 U of Taq DNA polymerase), and 5 μL of 4× Buffer Mix (final, 0.8 mM dNTPs, 50 mM KCl, 3.5 mM MgCl₂) (Nanohelix, Korea) was mixed with the third target nucleic acid (Tube 5:2.548 pg of UP genomic DNA) or distilled water (Tube 6; negative control) to prepare a final 20 μL reaction mixture.

Each of the reaction mixtures as prepared was placed in different tubes in a real-time thermocycler (CFX96 Real-time Cycler, Bio-Rad) and subjected to a real-time PCR consisting of denaturation at 95° C. for 15 minutes, and 50 cycles of 15 sec at 60° C., 5 sec at 62° C., 5 sec at 72° C., 5 sec at 76° C., 5 sec at 79° C., and 10 sec at 95° C.

Then, signals were measured at a first temperature (60° C.), a second temperature (62° C.), a third temperature (70° C.), a fourth temperature (76° C.), and a fifth temperature (79° C.) in each cycle to obtain amplification curves at each temperature. Then, RFU 300 was applied as a threshold to the amplification curves at each temperature, thereby obtaining Ct (cycle threshold) values.

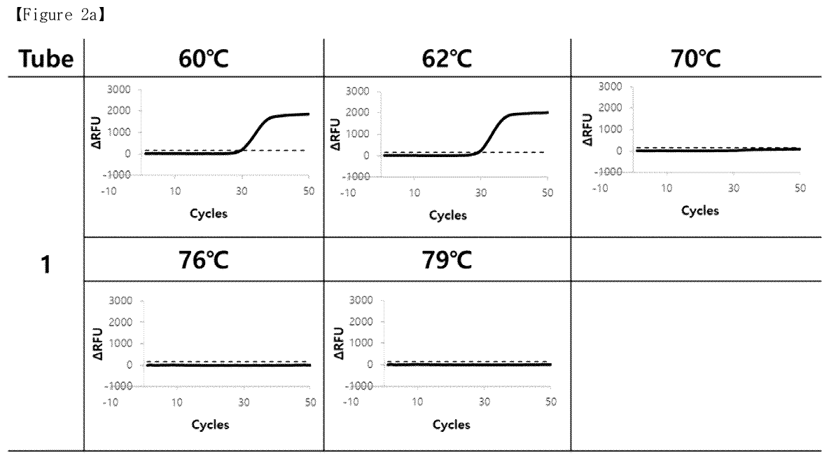

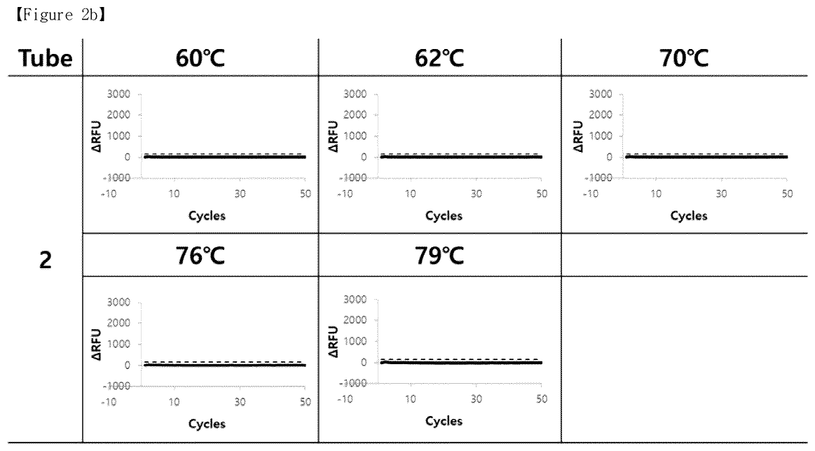

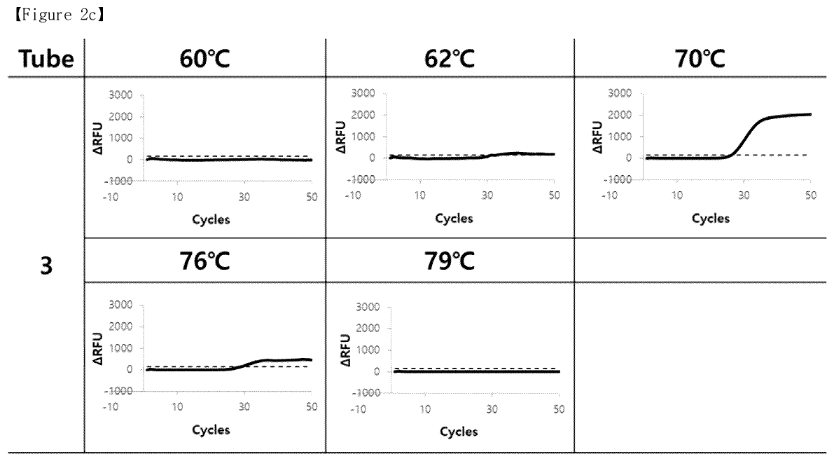

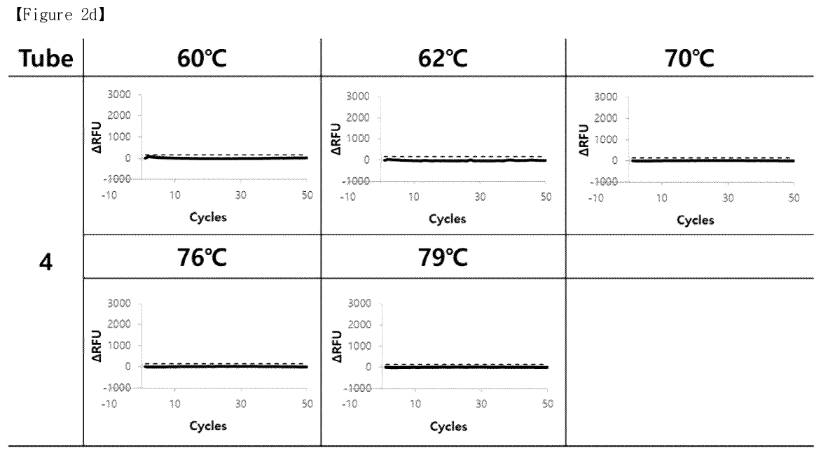

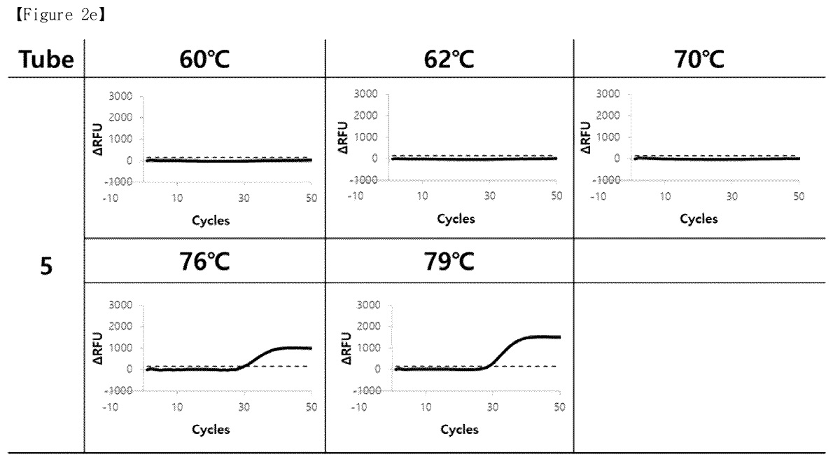

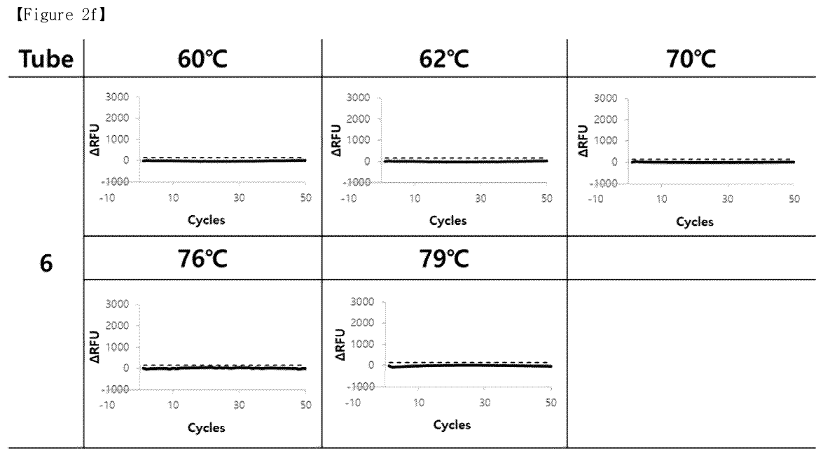

The amplification curves at 60° C., 62° C., 70° C., 76° C., and 79° C., and the Ct values obtained therefrom for Tubes 1 to 6 are shown in FIGS. 2A to 2F and Table 2.

TABLE 2

| | Ct (Cycle threshold) | | | | |
|---|---|---|---|---|---|
| | First | Second | Third | Fourth | Fifth |
| | temp. | temp. | temp. | temp. | temp. |
| Composition | (60° C.) | (62° C.) | (70° C.) | (76° C.) | (79° C.) |
| Tube 1 | 26.00 | 26.88 | N/A | N/A | N/A |
| (First composition) | | | | | |
| Tube 2 | N/A | N/A | N/A | N/A | N/A |
| (First composition) | | | | | |
| Tube 3 | N/A | 29.75 | 23.78 | 26.50 | N/A |
| (Second composition) | | | | | |
| Tube 4 | N/A | N/A | N/A | N/A | N/A |
| (Second composition) | | | | | |
| Tube 5 | N/A | N/A | N/A | 27.88 | 26.62 |
| (Third composition) | | | | | |
| Tube 6 | N/A | N/A | N/A | N/A | N/A |
| (Third composition) | | | | | |

Tube 1: 1.604 pg of CT genomic DNA
Tube 2: negative control
Tube 3: 9.498 pg of NG genomic DNA
Tube 4: negative control
Tube 5: 2.548 pg of UP genomic DNA
Tube 6: negative control
N/A: Not Applicable

As shown in Table 2 and FIGS. 2A to 2F, the first composition provided significant signals at the first temperature (60° C.) and a second temperature (62° C.) in the presence of the first target nucleic acid (see Tube 1). The second composition provided significant signals at the second temperature (62° C.), the third temperature (70° C.), and the fourth temperature (76° C.) in the presence of the second target nucleic acid (see Tube 3). The third composition provided significant signals at the fourth temperature (76° C.) and the fifth temperature (79° C.) in the presence of the third target nucleic acid (see Tube 5).

### Example 2: Determination of the Presence of Three Target Nucleic Acids Using Three Detection Temperatures (I)

It was investigated whether the presence of three target nucleic acids could be determined by signal measurement at three detection temperatures using the three compositions prepared in Example 1.

All the first to third compositions prepared in Example 1 were added to each tube containing the following target nucleic acids to prepare final 20 μL reaction mixtures, respectively:
Tube 1:1.604 pg of CT genomic DNA;
Tube 2:1.604 pg of CT genomic DNA and 9.498 pg of NG genomic DNA;
Tube 3:2.548 pg of UP genomic DNA;
Tube 4:9.498 pg of NG genomic DNA and 2.548 pg of UP genomic DNA;
Tube 5: negative control

Each tube was subjected to a PCR reaction, during which signals were measured at 62° C. (second temperature in Example 1), 70° C. (third detection in Example 1), and 79° C. (fifth temperature in Example 1) in every cycle using a single detection channel to obtain amplification curves at the three temperatures. Then, RFU 300 was applied as a threshold to the amplification curves at the three temperature, thereby obtaining Ct (cycle threshold) values.

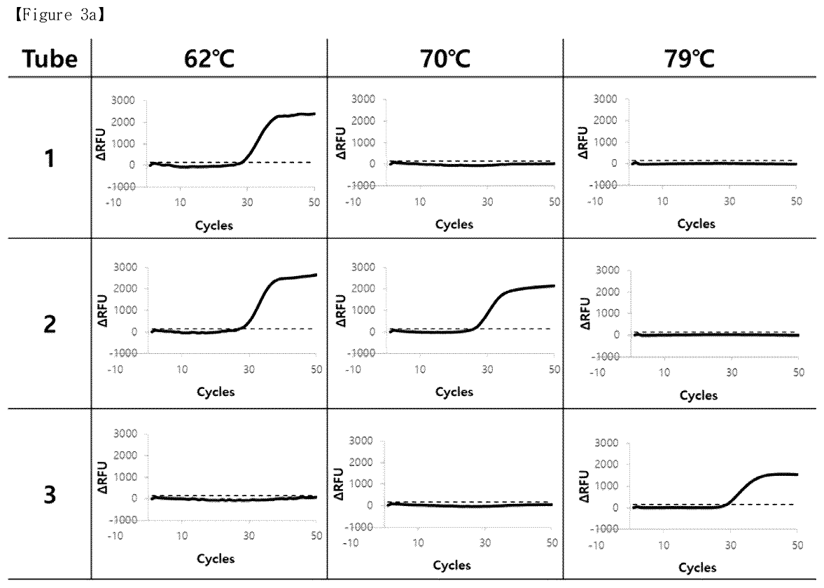

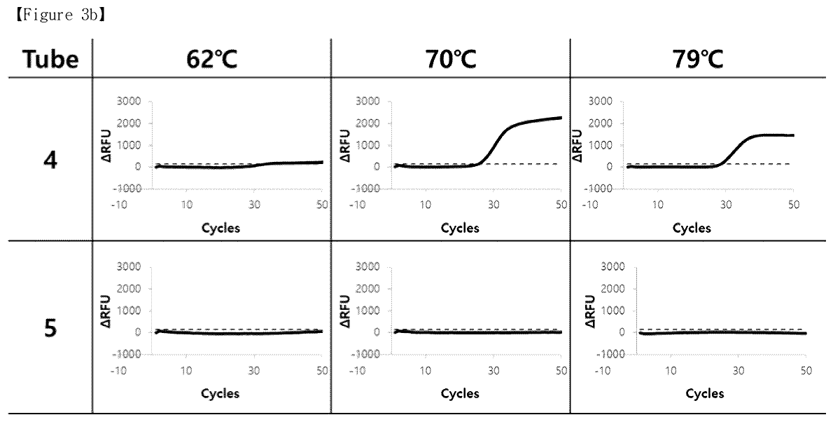

The amplification curves at 62° C., 70° C., and 79° C., and the Ct values obtained therefrom for Tubes 1 to 5 are shown in FIGS. 3A and 3B, and Table 3.

TABLE 3

| | Ct (Cycle threshold) | | |
|---|---|---|---|
| | First temperature | Second temperature | Third temperature |
| Composition | (62° C.) | (70° C.) | (79° C.) |
| Tube 1 | 25.97 | N/A | N/A |
| Tube 2 | 24.67 | 23.58 | N/A |
| Tube 3 | N/A | N/A | 27.00 |
| Tube 4 | N/A | 23.09 | 26.20 |
| Tube 5 | N/A | N/A | N/A |

Tube 1: CT genomic DNA
Tube 2: CT genomic DNA and NG genomic DNA
Tube 3: UP genomic DNA
Tube 4: NG genomic DNA and UP genomic DNA
Tube 5: negative control
N/A: Not Applicable

As shown in Table 3 and FIGS. 3A and 3B, significant signals were detected only at 62° C. for Tube 1 containing CT, at 62° C. and 70° C. for Tube 2 containing CT and NG, only at 79° C. for Tube 3 containing UP, at 70° C. and 79° C. for Tube 4 containing NG and UP. For Tube 5 containing no target nucleic acid, a significant signal was not detected at any of the three temperatures.

According to the principle of this Example, both the signal for the first target nucleic acid CT and the signal for the second target nucleic acid NG can be detected at the detection temperature of 62° C.; the signal for the second target nucleic acid NG alone can be detected at the detection temperature of 70° C.; the signal for the third target nucleic acid UP alone can be detected at the detection temperature of 79° C.

Accordingly, the signal measured at 70° C. was considered to be the signal of the second target nucleic acid NG and the signal measured at 79° C. was considered to be the signal of the third target nucleic acid UP. Meanwhile, the signal of the first target nucleic acid CT was obtained by extracting it from the signal measured at 62° C., using the signals measured at 62° C. and 70° C. and a reference value of NG, according to the signal extraction process as disclosed in U.S. Patent Application Publication No. 2017-0247750. Specifically, the difference (ratio) between the signal at 62° C. and the signal at 70° C. was calculated. Then, the End-Ratio between the signal at 62° C. and the signal at 70° C. in the presence of NG as a reference value was calculated to be 0.089 in Example 1. Afterwards, the following formula was used for each cycle to eliminate a signal for the NG at 62° C., thereby extracting only the signal for the CT:

$$Signal at 62 °C . - ( End - Ratio * Signal at 70 °C . )$$

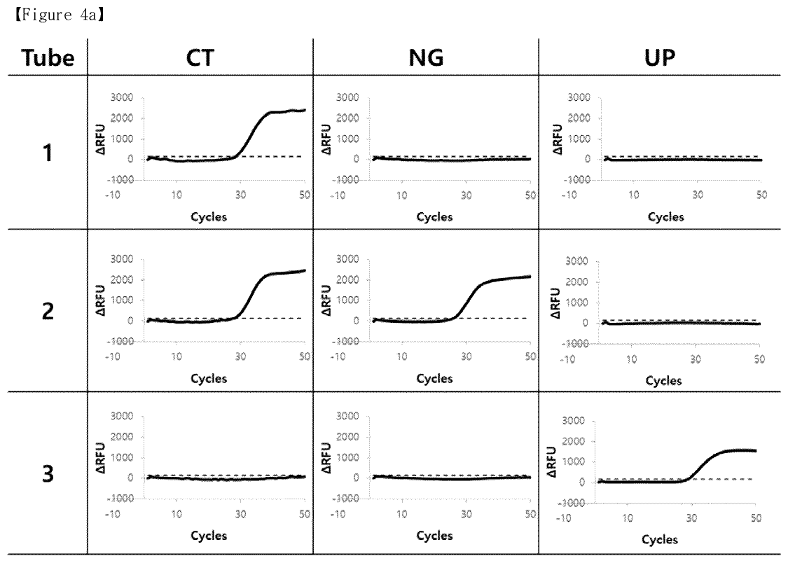

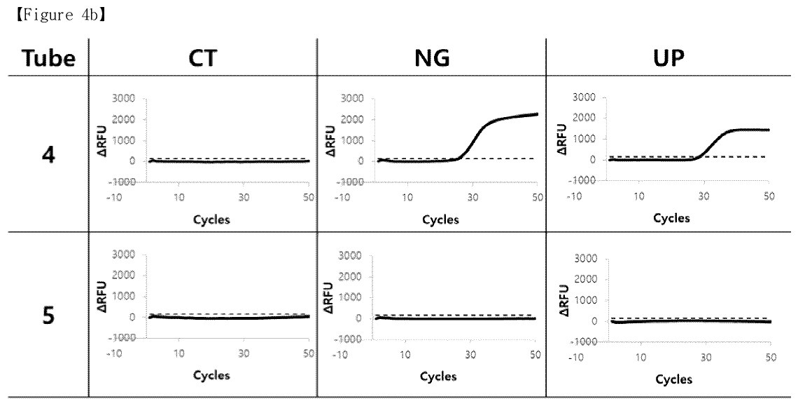

The signals (amplification curves) of each target nucleic acid for Tubes 1 to 5 as obtained above were shown in FIGS. 4A and 4B.

As shown in FIGS. 4A and 4B, for tube 1, the presence of CT, the absence of NG, and the absence of CT were determined from each amplification curve; for tube 2, the presence of CT, the presence of NG, and the absence of CT were determined from each amplification curve; for tube 3, the absence of CT, the absence of NG, and the presence of UP were determined; for tube 4, the absence of CT, the presence of NG, and the presence of UP were determined; for tube 5, the absence of CT, NG and UP was determined.

The Ct (cycle threshold) values obtained by applying RFU 150 as a threshold to the signals for each target nucleic acid are shown in Table 4.

TABLE 4

| | Ct (Cycle threshold) | | |
|---|---|---|---|
| | First temperature | Second temperature | Third temperature |
| Composition | (62° C.) | (70° C.) | (79° C.) |
| Tube 1 | 25.97 | N/A | N/A |
| Tube 2 | 25.10 | 23.58 | N/A |
| Tube 3 | N/A | N/A | 27.00 |
| Tube 4 | N/A | 23.09 | 26.20 |
| Tube 5 | N/A | N/A | N/A |

Tube 1: CT genomic DNA
Tube 2: CT genomic DNA and NG genomic DNA
Tube 3: UP genomic DNA
Tube 4: NG genomic DNA and UP genomic DNA
Tube 5: negative control
N/A: Not Applicable

The Ct values shown in Table 4 did not differ significantly from the Ct values for single target nucleic acids shown in Table 2. Therefore, it was confirmed that the presence of three target nucleic acids can be determined using three detection temperatures by the method of the present disclosure.

### Example 3: Determination of the Presence of Three Target Nucleic Acids Using Three Detection Temperatures (II)

It was investigated whether the presence of three target nucleic acids could be determined by signal measurement at three detection temperatures using the three compositions prepared in Example 2.

For this, the PCR reaction was performed in the same manner as Example 2, except that signals were measured at 60° C. (first detection temperature in Example 1), 70° C. (third detection temperature in Example 1), and 76° C. (fourth detection temperature in Example 1) every cycle.

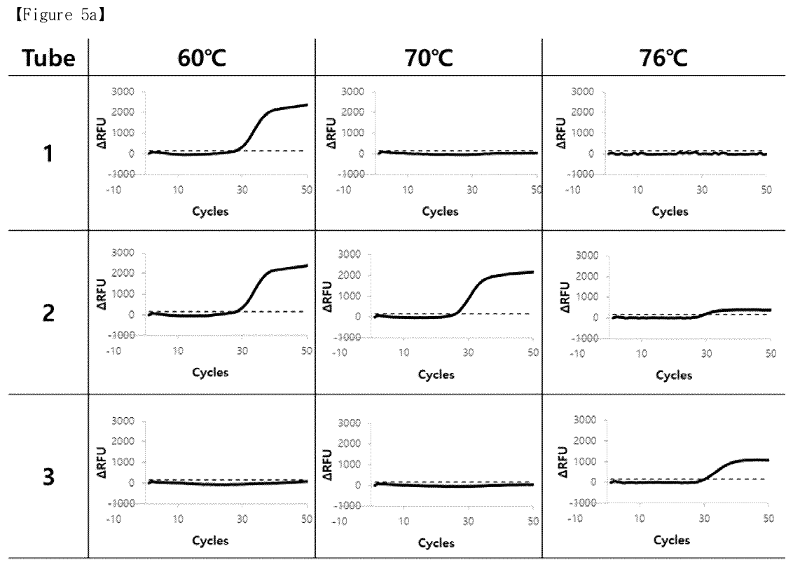

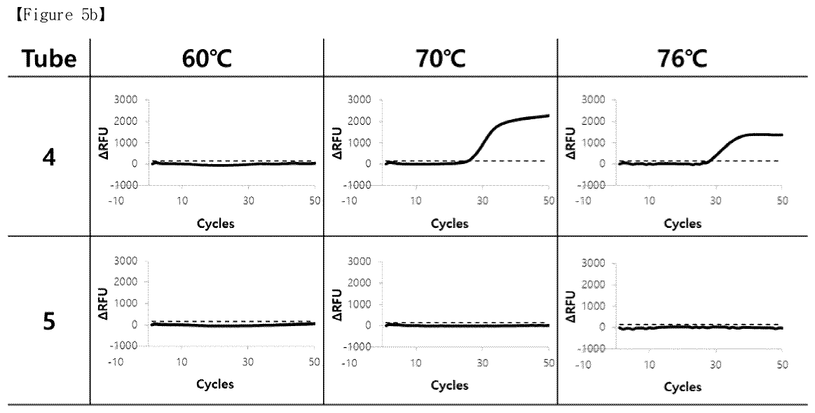

The amplification curves at 60° C., 70° C., and 76° C., and the Ct values obtained therefrom for Tubes 1 to 5 are shown in FIGS. 5A and 5B, and Table 5.

TABLE 5

| | Ct (Cycle threshold) | | |
|---|---|---|---|
| | First temperature | Second temperature | Third temperature |
| Composition | (60° C.) | (70° C.) | (76° C.) |
| Tube 1 | 26.00 | N/A | N/A |
| Tube 2 | 24.93 | 23.58 | N/A |
| Tube 3 | N/A | N/A | 27.87 |
| Tube 4 | N/A | 23.09 | 25.96 |
| Tube 5 | N/A | N/A | N/A |

Tube 1: CT genomic DNA
Tube 2: CT genomic DNA and NG genomic DNA
Tube 3: UP genomic DNA
Tube 4: NG genomic DNA and UP genomic DNA
Tube 5: negative control
N/A: Not Applicable

As shown in Table 5 and FIGS. 5A and 5B, significant signals were detected only at 60° C. for Tube 1 containing CT, at 60° C., 70° C., and 76° C. for Tube 2 containing CT and NG, only at 76° C. for Tube 3 containing UP, and at 70° C. and 76° C. for Tube 4 containing NG and UP. For Tube 5 containing no target, no significant signal was detected at all three temperatures.

According to the principle of this Example, the signal for the first target nucleic acid CT can be detected at the detection temperature of 60° C.; the signal for the second target nucleic acid NG can be detected at the detection temperature of 70° C.; and both the signal for the second target nucleic acid NG and the signal for the third target nucleic acid UP can be detected at the detection temperature of 76° C.

Accordingly, the signal measured at 60° C. was considered to be the signal of the first target nucleic acid CT and the signal measured at 70° C. was considered to be the signal of the second target nucleic acid NG. Meanwhile, the signal of the third target nucleic acid UP was obtained by extracting it from the signal measured at 76° C., using the signals measured at 70° C. and 76° C. and a reference value of NG, according to the signal extraction process as disclosed in U.S. Patent Application Publication No. 2019-0024155. Specifically, the difference (ratio) between the signal at 76° C. and the signal at 70° C. was calculated. Then, the End-Ratio between the signal at 76° C. and the signal at 70° C. in the presence of NG as a reference value was calculated to be 0.225 in Example 1. Afterwards, the following formula was used for each cycle to eliminate a signal for NG at 76° C., thereby extracting only the signal for the UP:

$$Signal at 76 °C . - ( End - Ratio * Signal at 70 °C . )$$

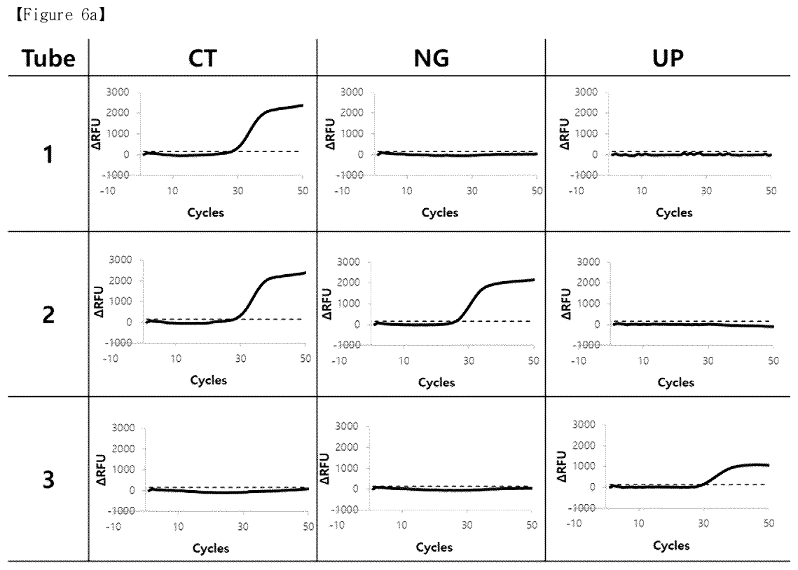

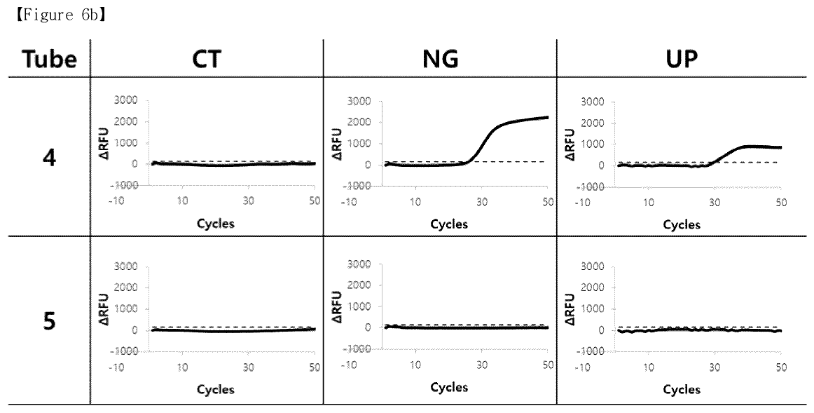

The signals (amplification curves) of each target nucleic acid for Tubes 1 to 5 were shown in FIGS. 6A and 6B.

As shown in FIGS. 6A and 6B, for tube 1, the presence of CT, the absence of NG, and the absence of UP were determined from each amplification curve; for tube 2, the presence of CT, the presence of NG, and the absence of UP were determined from each amplification curve; for tube 3, the absence of CT, the absence of NG, and the presence of UP were determined from each amplification curve; for tube 4, the absence of CT, the presence of NG, and the presence of UP were determined from each amplification curve; for tube 5, the absence of CT, NG and UP was determined.

The Ct (cycle threshold) values obtained by applying RFU 150 as a threshold to the extracted signal for each target nucleic acid are shown in Table 6.

TABLE 6

| | Ct (Cycle threshold) | | |
|---|---|---|---|
| | First temperature | Second temperature | Third temperature |
| Composition | (60° C.) | (70° C.) | (76° C.) |
| Tube 1 | 26.00 | N/A | N/A |
| Tube 2 | 24.93 | 23.58 | N/A |
| Tube 3 | N/A | N/A | 27.87 |
| Tube 4 | N/A | 23.09 | 27.55 |
| Tube 5 | N/A | N/A | N/A |

Tube 1: CT genomic DNA
Tube 2: CT genomic DNA and NG genomic DNA
Tube 3: UP genomic DNA
Tube 4: NG genomic DNA and UP genomic DNA
Tube 5: negative control
N/A: Not Applicable

The Ct values shown in Table 6 did not differ significantly from the Ct values for single target nucleic acids shown in Table 2. Therefore, it was confirmed that the presence of three target nucleic acids can be determined using three detection temperatures by the method of the present disclosure.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for detecting n target nucleic acids in a sample using n detection temperatures, comprising the steps of:
(a) reacting, in a single reaction vessel, a sample suspected of containing at least one of the n target nucleic acids with n compositions for detecting the n target nucleic acids,
wherein n is an integer of 3 or more,
wherein, during the reacting, the n target nucleic acids in the sample are amplified,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are each configured to provide a signal indicative of the presence of its corresponding target nucleic acid only at one different detection temperature among the n detection temperatures in the presence of the corresponding target nucleic acid, whereas the other compositions are each configured to provide a signal indicative of the presence of its corresponding target nucleic acid at two to n adjacent detection temperatures among the n detection temperatures arranged in order in the presence of the corresponding target nucleic acid,
wherein at each of the n detection temperatures, signals are provided by one or two of the n compositions;
(b) measuring the signals at the n detection temperatures under the single detection channel,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(c) determining the presence of a target nucleic acid from the single signal measured at the single-signal detection temperature and determining the presence of a target nucleic acid from an extracted single signal, which is obtained by extraction from the combined signal measured at the combined-signal detection temperature, whereby the presence of the n target nucleic acids are determined from the signals measured at the n detection temperatures.

2. The method of claim 1, wherein the reacting is real-time PCR.

3. The method of claim 1, wherein at least one of the n compositions provides a signal in a manner dependent on formation or dissociation of a duplex.

4. The method of claim 1, wherein at least one of the n compositions provides a signal by formation of a duplex in a manner dependent on cleavage of a mediation oligonucleotide specifically hybridized to its corresponding target nucleic acid.

5. The method of claim 1, wherein at least one of the n compositions provides a signal in a manner dependent on cleavage of an oligonucleotide specifically hybridized to its corresponding target nucleic acid.

6. The method of claim 1, wherein each of the n compositions provides a signal in a predetermined temperature range but does not provide a signal in other temperature ranges.

7. The method of claim 1, wherein n is 3.

8. The method of claim 7, wherein (i) one of the three compositions is configured to provide a signal indicative of the presence of its corresponding target nucleic acid at only one of the three detection temperatures arranged in order, and wherein each of the two compositions for detecting target nucleic acids is configured to provide a signal indicative of the presence of its corresponding target nucleic acid at two adjacent detection temperatures of the three detection temperatures arranged in order; (ii) one of three compositions is configured to provide a signal indicative of the presence of its corresponding target nucleic acid only at one detection temperature among three detection temperatures arranged in order, another composition is configured to provide a signal indicative of the presence of its corresponding target nucleic acid at two adjacent detection temperatures among the three detection temperatures arranged in order, and the other composition is configured to provide a signal indicative of the presence of its corresponding target nucleic acid at the three detection temperatures arranged in order; or (iii) two of three compositions are each configured to provide a signal indicative of the presence of its corresponding target nucleic acid only at one different detection temperature among three detection temperatures arranged in order, and the other one composition is configured to provide a signal indicative of the presence of its corresponding target nucleic acid at two or three adjacent detection temperatures among the three detection temperatures arranged in order.

9. (canceled)

10. (canceled)

11. The method of claim 1, wherein n is 4.

12. The method of claim 11, wherein (i) one of four compositions is configured to provide a signal indicative of the presence of its corresponding target nucleic acid only at one detection temperature among four detection temperatures arranged in order, and the other three compositions are each configured to provide a signal indicative of the presence of its corresponding target nucleic acid at two adjacent detection temperatures among the four detection temperatures arranged in order; (ii) one of four compositions is configured to provide a signal indicative of the presence of its corresponding target nucleic acid only at one detection temperature among four detection temperatures arranged in order, another two compositions are each configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three adjacent detection temperatures among the four detection temperatures arranged in order; (iii) one of four compositions is configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other two compositions are each configured to provide a signal indicative of the presence of its corresponding target nucleic acid at three adjacent detection temperatures among the four detection temperatures arranged in order; (iv) two of four compositions are each configured to provide a signal indicative of the presence of its corresponding target nucleic acid only at one different detection temperature among four detection temperatures arranged in order, and the other two compositions are each configured to provide a signal indicative of the presence of its corresponding target nucleic acid at two or three adjacent detection temperatures among the four detection temperatures arranged in order; (v) two of four compositions are each configured to provide a signal indicative of the presence of its corresponding target nucleic acid only at one different detection temperature among four detection temperatures arranged in order, another one composition is configured to provide a signal indicative of the presence of its corresponding target nucleic acid at two adjacent detection temperatures among the four detection temperatures arranged in order, and the other one composition is configured to provide a signal indicative of the presence of its corresponding target nucleic acid at three or four adjacent detection temperatures among the four detection temperatures arranged in order; or (vi) three of four compositions are each configured to provide a signal indicative of the presence of its corresponding target nucleic acid only at one different detection temperature among four detection temperatures arranged in order, and the other one composition is configured to provide a signal indicative of the presence of its corresponding target nucleic acid at two to four adjacent detection temperatures among the four detection temperatures arranged in order.

13. (canceled)

14. (canceled)

15. (canceled)

16. (canceled)

17. (canceled)

18. The method of claim 1, wherein n is 5.

19. The method of claim 18, wherein (i) one of five compositions is configured to provide a signal indicative of the presence of its corresponding target nucleic acid only at one detection temperature among five detection temperatures arranged in order, and the other four compositions are each configured to provide a signal indicative of the presence of its corresponding target nucleic acid at two to five adjacent detection temperatures among the five detection temperatures arranged in order; (ii) two of five compositions are each configured to provide a signal indicative of the presence of its corresponding target nucleic acid only at one different detection temperature among five detection temperatures arranged in order, and the other three compositions are each configured to provide a signal indicative of the presence of its corresponding target nucleic acid at two to five adjacent detection temperatures among the five detection temperatures arranged in order; (iii) three of five compositions are each configured to provide a signal indicative of the presence of its corresponding target nucleic acid only at one different detection temperature among five detection temperatures arranged in order, and the other two compositions are each configured to provide a signal indicative of the presence of its corresponding target nucleic acid at two to five adjacent detection temperatures among the five detection temperatures arranged in order; or (iv) four of five compositions are each configured to provide a signal indicative of the presence of its corresponding target nucleic acid only at one different detection temperature among five detection temperatures arranged in order, and the other one composition is configured to provide a signal indicative of the presence of its corresponding target nucleic acid at two to five adjacent detection temperatures among the five detection temperatures arranged in order.

20. The method of claim 1, wherein two of the n compositions do not provide a signal indicative of the presence of its corresponding target nucleic acids at two or more identical detection temperatures.

21. The method of claim 1, wherein the number of the combined-signal detection temperatures is n−2 or less.

22. The method of claim 1, wherein the extraction of a single signal from the combined signal measured at the combined-signal detection temperature is performed by using (i) the combined signal measured at the combined-signal detection temperature and (ii) a single signal measured at a single-signal detection temperature, which is provided by a composition also providing a signal at the combined signal detection temperature.

23. The method of claim 1, wherein the extraction of a single signal from the combined signal measured at the combination-signal detection temperature is performed by eliminating a signal provided by another composition from the combined signal measured at the combination-signal detection temperature.

24. The method of claim 1, wherein the extraction of a single signal from the combined signal measured at the combined-signal detection temperature is performed by (i) the difference between the combined signal measured at the combination-signal detection temperature and a single signal provided by another composition at a single-signal detection temperature and (ii) a reference value.

25. The method of claim 24, wherein the reference value is a value that reflects the change in signals provided by a composition at two detection temperatures in the presence of its corresponding target nucleic acid.

26. The method of claim 25, wherein the reference value is obtained by (i) reacting a composition with a sample containing its corresponding target nucleic acid in a reaction vessel different from the reaction vessel of step (a) to amplify the target nucleic acid in the sample, and (ii) measuring signals at two detection temperature, and (iii) calculating the difference between the signals.

27. (canceled)

28. A computer readable storage medium containing instructions to configure a processor to perform a method for detecting n target nucleic acids in a sample using n detection temperatures, the method comprising:
(a) receiving signals measured at the n detection temperatures under a single detection channel,
wherein the signals are obtained by reacting, in a single reaction vessel, a sample suspected of containing at least one of n target nucleic acids with n compositions for detecting n target nucleic acids, and measuring the signals at the n detection temperatures under the single detection channel,
wherein n is an integer of 3 or more,
wherein, during the reacting, the n target nucleic acids in the sample are amplified,
wherein each of the n compositions comprises one or more oligonucleotides capable of providing a signal depending on the presence of its corresponding target nucleic acid among the n target nucleic acids, and signals provided by the n compositions are not differentiated from each other by a single detection channel,
wherein 1 to n−1 of the n compositions are configured to provide a signal indicative of the presence of its corresponding target nucleic acid only at one different detection temperature among the n detection temperatures in the presence of the corresponding target nucleic acid, whereas the other compositions are configured to provide a signal indicative of the presence of its corresponding target nucleic acid at two to n adjacent detection temperatures among the n detection temperatures arranged in order in the presence of the corresponding target nucleic acid,
wherein none to n−1 of the n detection temperatures are single-signal detection temperatures selected such that a single signal is provided by one of the n compositions, whereas the other detection temperatures are combined-signal detection temperatures selected such that a combined signal is provided by two of the n compositions; and
(b) determining the presence of a target nucleic acid from the single signal measured at the single-signal detection temperature and determining the presence of a target nucleic acid from a single signal, which is extracted from the combined signal measured at the combined-signal detection temperature such that the presence of the n target nucleic acids are determined from the signals measured at the n detection temperatures.

29. (canceled)

30. (canceled)
